SEQUENCE LISTING (18 sequences)

SEQ ID NO: 1 - DNA, 5490 bp, Human
  CDS (118)...(4518)
ggctgagttttatgacgggcccggtgctgaagggcagggaacaacttgatggtgctactttgaactgcttttcttttctcctttttgcacaaagagtctcatgtctgatatttagacatgatgagctttgtgcaaaaggggagctggctacttctcgctctgcttcatcccactattattttggcacaacaggaagctgttgaaggaggatgttcccatcttggtcagtcctatgcggatagagatgtctggaagccagaaccatgccaaatatgtgtctgtgactcaggatccgttctctgcgatgacataatatgtgacgatcaagaattagactgccccaacccagaaattccatttggagaatgttgtgcagtttgcccacagcctccaactgctcctactcgccctcctaatggtcaaggacctcaaggccccaagggagatccaggccctcctggtattcctgggagaaatggtgaccctggtattccaggacaaccagggtcccctggttctcctggcccccctggaatctgtgaatcatgccctactggtcctcagaactattctccccagtatgattcatatgatgtcaagtctggagtagcagtaggaggactcgcaggctatcctggaccagctggccccccaggccctcccggtccccctggtacatctggtcatcctggttcccctggatctccaggataccaaggaccccctggtgaacctgggcaagctggtccttcaggccctccaggacctcctggtgctataggtccatctggtcctgctggaaaagatggagaatcaggtagacccggacgacctggagagcgaggattgcctggacctccaggtatcaaaggtccagctgggatacctggattccctggtatgaaaggacacagaggcttcgatggacgaaatggagaaaagggtgaaacaggtgctcctggattaaagggtgaaaatggtcttccaggcgaaaatggagctcctggacccatgggtccaagaggggctcctggtgagcgaggacggccaggacttcctggggctgcaggtgctcggggtaatgacggtgctcgaggcagtgatggtcaaccaggccctcctggtcctcctggaactgccggattccctggatcccctggtgctaagggtgaagttggacctgcagggtctcctggttcaaatggtgcccctggacaaagaggagaacctggacctcagggacacgctggtgctcaaggtcctcctggccctcctgggattaatggtagtcctggtggtaaaggcgaaatgggtcccgctggcattcctggagctcctggactgatgggagcccggggtcctccaggaccagccggtgctaatggtgctcctggactgcgaggtggtgcaggtgagcctggtaagaatggtgccaaaggagagcccggaccacgtggtgaacgcggtgaggctggtattccaggtgttccaggagctaaaggcgaagatggcaaggatggatcacctggagaacctggtgcaaatgggcttccaggagctgcaggagaaaggggtgcccctgggttccgaggacctgctggaccaaatggcatcccaggagaaaagggtcctgctggagagcgtggtgctccaggccctgcagggcccagaggagctgctggagaacctggcagagatggcgtccctggaggtccaggaatgaggggcatgcccggaagtccaggaggaccaggaagtgatgggaaaccagggcctcccggaagtcaaggagaaagtggtcgaccaggtcctcctgggccatctggtccccgaggtcagcctggtgtcatgggcttccccggtcctaaaggaaatgatggtgctcctggtaagaatggagaacgaggtggccctggaggacctggccctcagggtcctcctggaaagaatggtgaaactggacctcagggacccccagggcctactgggcctggtggtgacaaaggagacacaggaccccctggtccacaaggattacaaggcttgcctggtacaggtggtcctccaggagaaaatggaaaacctggggaaccaggtccaaagggtgatgccggtgcacctggagctccaggaggcaagggtgatgctggtgcccctggtgaacgtggacctcctggattggcaggggccccaggacttagaggtggagctggtccccctggtcccgaaggaggaaagggtgctgctggtcctcctgggccacctggtgctgctggtactcctggtctgcaaggaatgcctggagaaagaggaggtcttggaagtcctggtccaaagggtgacaagggtgaaccaggcggtccaggtgctgatggtgtcccagggaaagatggcccaaggggtcctactggtcctattggtcctcctggcccagctggccagcctggagataagggtgaaggtggtgcccccggacttccaggtatagctggacctcgtggtagccctggtgagagaggtgaaactggccctccaggacctgctggtttccctggtgctcctggacagaatggtgaacctggtggtaaaggagaaagaggggctccgggtgagaaaggtgaaggaggccctcctggagttgcaggaccccctggaggttctggacctgctggtcctcctggtccccaaggtgtcaaaggtgaacgtggcagtcctggtggacctggtgctgctggcttccctggtgctcgtggtcttcctggtcctcctggtagtaatggtaacccaggacccccaggtcccagcggttctccaggcaaggatgggcccccaggtcctgcgggtaacactggtgctcctggcagccctggagtgtctggaccaaaaggtgatgctggccaaccaggagagaagggatcgcctggtgcccagggcccaccaggagctccaggcccacttgggattgctgggatcactggagcacggggtcttgcaggaccaccaggcatgccaggtcctaggggaagccctggccctcagggtgtcaagggtgaaagtgggaaaccaggagctaacggtctcagtggagaacgtggtccccctggaccccagggtcttcctggtctggctggtacagctggtgaacctggaagagatggaaaccctggatcagatggtcttccaggccgagatggatctcctggtggcaagggtgatcgtggtgaaaatggctctcctggtgcccctggcgctcctggtcatccaggcccacctggtcctgtcggtccagctggaaagagtggtgacagaggagaaagtggccctgctggccctgctggtgctcccggtcctgctggttcccgaggtgctcctggtcctcaaggcccacgtggtgacaaaggtgaaacaggtgaacgtggagctgctggcatcaaaggacatcgaggattccctggtaatccaggtgccccaggttctccaggccctgctggtcagcagggtgcaatcggcagtccaggacctgcaggccccagaggacctgttggacccagtggacctcctggcaaagatggaaccagtggacatccaggtcccattggaccaccagggcctcgaggtaacagaggtgaaagaggatctgagggctccccaggccacccagggcaaccaggccctcctggacctcctggtgcccctggtccttgctgtggtggtgttggagccgctgccattgctgggattggaggtgaaaaagctggcggttttgccccgtattatggagatgaaccaatggatttcaaaatcaacaccgatgagattatgacttcactcaagtctgttaatggacaaatagaaagcctcattagtcctgatggttctcgtaaaaaccccgctagaaactgcagagacctgaaattctgccatcctgaactcaagagtggagaatactgggttgaccctaaccaaggatgcaaattggatgctatcaaggtattctgtaatatggaaactggggaaacatgcataagtgccaatcctttgaatgttccacggaaacactggtggacagattctagtgctgagaagaaacacgtttggtttggagagtccatggatggtggttttcagtttagctacggcaatcctgaacttcctgaagatgtccttgatgtgcagctggcattccttcgacttctctccagccgagcttcccagaacatcacatatcactgcaaaaatagcattgcatacatggatcaggccagtggaaatgtaaagaaggccctgaagctgatggggtcaaatgaaggtgaattcaaggctgaaggaaatagcaaattcacctacacagttctggaggatggttgcacgaaacacactggggaatggagcaaaacagtctttgaatatcgaacacgcaaggctgtgagactacctattgtagatattgcaccctatgacattggtggtcctgatcaagaatttggtgtggacgttggccctgtttgctttttataaaccaaactctatctgaaatcccaacaaaaaaaatttaactccatatgtgttcctcttgttctaatcttgtcaaccagtgcaagtgaccgacaaaattccagttatttatttccaaaatgtttggaaacagtataatttgacaaagaaaaatgatacttctctttttttgctgttccaccaaatacaattcaaatgctttttgttttatttttttaccaattccaatttcaaaatgtctcaatggtgctataataaataaacttcaacactctttatgataacaacactgtgttatattctttgaatcctagcccatctgcagagcaatgactgtgctcaccagtaaaagataacctttctttctgaaatagtcaaatacgaaattagaaaagccctccctattttaactacctcaactggtcagaaacacagattgtattctatgagtcccagaagatgaaaaaaattttatacgttgataaaacttataaatttcattgattaatctcctggaagattggtttaaaaagaaaagtgtaatgcaagaatttaaagaaatatttttaaagccacaattattttaatattggatatcaactgcttgtaaaggtgctcctcttttttcttgtcattgctggtcaagattactaatatttgggaaggctttaaagacgcatgttatggtgctaatgtactttcacttttaaactctagatcagaattgttgacttgcattcagaacataaatgcacaaaatctgtacatgtctcccatcagaaagattcattggcatgccacaggggattctcctccttcatcctgtaaaggtcaacaataaaaaccaaattatggggctgcttttgtcacactagcatagagaatgtgttgaaatttaactttgtaagcttgtatgtggttgttgatcttttttttccttacagacacccataataaaatatcatattaaaattc

SEQ ID NO: 2 - protein, 1466 aa, Human
MMSFVQKGSWLLLALLHPTIILAQQEAVEGGCSHLGQSYADRDVWKPEPCQICVCDSGSVLCDDIICDDQELDCPNPEIPFGECCAVCPQPPTAPTRPPNGQGPQGPKGDPGPPGIPGRNGDPGIPGQPGSPGSPGPPGICESCPTGPQNYSPQYDSYDVKSGVAVGGLAGYPGPAGPPGPPGPPGTSGHPGSPGSPGYQGPPGEPGQAGPSGPPGPPGAIGPSGPAGKDGESGRPGRPGERGLPGPPGIKGPAGIPGFPGMKGHRGFDGRNGEKGETGAPGLKGENGLPGENGAPGPMGPRGAPGERGRPGLPGAAGARGNDGARGSDGQPGPPGPPGTAGFPGSPGAKGEVGPAGSPGSNGAPGQRGEPGPQGHAGAQGPPGPPGINGSPGGKGEMGPAGIPGAPGLMGARGPPGPAGANGAPGLRGGAGEPGKNGAKGEPGPRGERGEAGIPGVPGAKGEDGKDGSPGEPGANGLPGAAGERGAPGFRGPAGPNGIPGEKGPAGERGAPGPAGPRGAAGEPGRDGVPGGPGMRGMPGSPGGPGSDGKPGPPGSQGESGRPGPPGPSGPRGQPGVMGFPGPKGNDGAPGKNGERGGPGGPGPQGPPGKNGETGPQGPPGPTGPGGDKGDTGPPGPQGLQGLPGTGGPPGENGKPGEPGPKGDAGAPGAPGGKGDAGAPGERGPPGLAGAPGLRGGAGPPGPEGGKGAAGPPGPPGAAGTPGLQGMPGERGGLGSPGPKGDKGEPGGPGADGVPGKDGPRGPTGPIGPPGPAGQPGDKGEGGAPGLPGIAGPRGSPGERGETGPPGPAGFPGAPGQNGEPGGKGERGAPGEKGEGGPPGVAGPPGGSGPAGPPGPQGVKGERGSPGGPGAAGFPGARGLPGPPGSNGNPGPPGPSGSPGKDGPPGPAGNTGAPGSPGVSGPKGDAGQPGEKGSPGAQGPPGAPGPLGIAGITGARGLAGPPGMPGPRGSPGPQGVKGESGKPGANGLSGERGPPGPQGLPGLAGTAGEPGRDGNPGSDGLPGRDGSPGGKGDRGENGSPGAPGAPGHPGPPGPVGPAGKSGDRGESGPAGPAGAPGPAGSRGAPGPQGPRGDKGETGERGAAGIKGHRGFPGNPGAPGSPGPAGQQGAIGSPGPAGPRGPVGPSGPPGKDGTSGHPGPIGPPGPRGNRGERGSEGSPGHPGQPGPPGPPGAPGPCCGGVGAAAIAGIGGEKAGGFAPYYGDEPMDFKINTDEIMTSLKSVNGQIESLISPDGSRKNPARNCRDLKFCHPELKSGEYWVDPNQGCKLDAIKVFCNMETGETCISANPLNVPRKHWWTDSSAEKKHVWFGESMDGGFQFSYGNPELPEDVLDVQLAFLRLLSSRASQNITYHCKNSIAYMDQASGNVKKALKLMGSNEGEFKAEGNSKFTYTVLEDGCTKHTGEWSKTVFEYRTRKAVRLPIVDIAPYDIGGPDQEFGVDVGPVCFL

SEQ ID NO: 3 - DNA, 3531 bp, Artificial Sequence
  CDS (1)...(3531)
  In silico generated modification of human sequence
ccagaaattccatttggagaatgttgtccaatttgtccaactgatttggctacagctagcggtcagcctggtccaaagggtcaaaaaggtgaacctggtgatataaaagacattgttggtcctaaaggtccacctggtcctcagggtcccgcaggagaacaaggacctagaggtgacaggggcgataaaggagaaaaaggagcacctggtccaaggggtagagatggtgaaccaggcacacctggtaatccaggtccgcccggcccacctggccctcctggtcctcccggtttgggtggtaattttgctgctcaaatggctggtggttttgatgaaaaagctggtggtgctcaattgggtgttatgcaaggtccaatgggacctatgggtcccaggggcccacccggtccagcaggagcccctggaccccaaggctttcaaggtaatccaggtgagcccggggaaccaggtgtatctggtccaatgggcccaaggggtcctcccgggcctcctggtaagcctggtgatgatggtgaagctggtaagccaggaaaagcaggtgagaggggacctcccggaccccaaggtgctcgtggctttccaggaactccaggtcttcccggtgttaaaggtcatagaggttatccaggtttggatggggctaaaggagaggctggagctccaggcgttaaaggagaatctggttctcctggtgaaaatggaagtccaggtccaatgggtcctagaggcttgcctggagaaagaggtagaactggtcccgctggtgctgctggggctaggggtaatgatggtcaacctggtcctgccggccctcccggaccagtaggaccagcaggtggtccaggttttccaggcgcacctggagctaaaggagaagctggtccaactggagcccgtggtccagaaggagctcaaggaccaaggggagaaccaggaactcctggttctcctggaccagctggtgcttctggaaatcctggaactgatggcattccaggtgctaaaggatctgcaggagctccaggaattgctggcgcacccggttttcctggacctaggggtccaccaggacctcagggtgcaactggtccattgggtccaaaaggtcaaactggtgaaccaggaatagctggttttaagggtgaacaaggaccaaaaggagaacctggtccagcaggtccacaaggtgcaccaggtcccgcgggagaagaaggtaaaagaggtgccaggggtgaaccaggaggtgttggtccaattggaccaccaggtgaaagaggtgctccaggtaatagaggttttcctggtcaagatggtttggctggtccaaaaggcgccccaggtgagagaggcccatctggcttggctggccctaaaggagctaatggtgatccaggtagaccaggagaacctggcctgccaggtgctagaggattgactggtagaccaggcgatgctggaccacaaggtaaggttggtccttctggtgctccaggtgaagatggtagacccggtcctccgggaccacaaggcgccaggggacaacctggtgttatgggttttccaggaccaaaaggtgctaatggtgaacctggtaaagctggtgagaaaggtttaccaggtgctcccggtttgagaggactacctggtaaagacggtgaaaccggtgctgctggtcctcctggcccagcgggaccagccggtgagagaggagaacagggtgccccgggtccttcaggttttcaaggattacccggaccaccaggaccaccgggcgagggtggaaaaccaggtgatcaaggagttccaggagaagcaggtgccccaggattggttggtcccagaggcgaacgtggctttcccggtgagaggggctctcctggcgcacaaggtttgcagggtcctaggggtctacctggtactccaggaactgatggtcccaaaggtgcttctggtccagctggtccacctggtgctcaaggaccacctggtttgcaaggcatgccaggtgaaaggggcgcagctggaattgcaggtccaaaaggagatagaggagatgttggcgaaaaaggtccagaaggtgctccaggcaaagacggaggtagaggtttgactggaccaataggaccaccaggcccagccggggcaaatggtgaaaaaggtgaagttggtcctcctggtcccgcaggttctgcaggtgcgcgtggggctccaggagaaaggggtgagacaggtccgccagggcctgcaggctttgcaggaccacctggcgcagatggtcaaccaggtgcaaaaggtgaacaaggcgaagcaggtcaaaaaggagatgctggtgccccaggaccacaaggcccatcgggtgccccaggcccacaaggtccaactggtgttactggtccaaaaggagctagaggtgctcagggtccaccaggagcaactggattccctggagctgctggcagagttggtccaccaggctctaatggaaacccaggacctcctggcccaccagggccgagtggtaaagacggacctaagggtgccagaggtgattctggtccgccaggtagagctggtgaacccggtttgcaaggcccagctggaccgccaggcgaaaaaggcgagccaggagatgatggccctagcggtgctgaaggccctccgggtccccagggcttggctggacaaagaggaattgttggtttaccagggcagaggggcgaacgtggttttccaggtttgccaggaccttcaggagagccaggtaaacaaggtgctcctggtgcttcaggcgatagaggtccaccaggcccggttggacctcccggattgactggcccagctggtgagccaggcagagaaggatcaccaggtgctgatggtccaccaggtagagatggtgctgctggagttaagggtgacagaggagaaactggtgctgttggtgcacctggcgcaccaggtcctcctggctctccgggccccgctggtcccacaggaaaacaaggagacaggggtgaagcaggtgctcagggaccaatgggaccatctggacccgctggagctagaggaatacagggtccacagggtcctagaggtgataaaggtgaagccggcgaacccggagaaagaggattaaaaggacatagaggttttactggtttgcaaggtctaccaggccctcctggaccttctggagatcaaggtgcatctggacctgcagggccttcaggacctagaggtccacctggcccagtaggtccttctggtaaagatggtgcaaatggaataccaggtccaattggtccacccggtcccagaggcagatctggtgaaactggtccagcaggccctcctggcaaccctggaccaccaggtccaccaggtccgccgggtccgggaatagatatgtctgcttttgctggtttgggtccaagggaaaaaggaccggatccattgcaatatatgagagctgatcaagctgctggtggtttgagacaacatgatgctgaa

SEQ ID NO: 4 - protein, 1177 aa, Artificial Sequence
  In silico generated modification of human sequence
PEIPFGECCPICPTDLATASGQPGPKGQKGEPGDIKDIVGPKGPPGPQGPAGEQGPRGDRGDKGEKGAPGPRGRDGEPGTPGNPGPPGPPGPPGPPGLGGNFAAQMAGGFDEKAGGAQLGVMQGPMGPMGPRGPPGPAGAPGPQGFQGNPGEPGEPGVSGPMGPRGPPGPPGKPGDDGEAGKPGKAGERGPPGPQGARGFPGTPGLPGVKGHRGYPGLDGAKGEAGAPGVKGESGSPGENGSPGPMGPRGLPGERGRTGPAGAAGARGNDGQPGPAGPPGPVGPAGGPGFPGAPGAKGEAGPTGARGPEGAQGPRGEPGTPGSPGPAGASGNPGTDGIPGAKGSAGAPGIAGAPGFPGPRGPPGPQGATGPLGPKGQTGEPGIAGFKGEQGPKGEPGPAGPQGAPGPAGEEGKRGARGEPGGVGPIGPPGERGAPGNRGFPGQDGLAGPKGAPGERGPSGLAGPKGANGDPGRPGEPGLPGARGLTGRPGDAGPQGKVGPSGAPGEDGRPGPPGPQGARGQPGVMGFPGPKGANGEPGKAGEKGLPGAPGLRGLPGKDGETGAAGPPGPAGPAGERGEQGAPGPSGFQGLPGPPGPPGEGGKPGDQGVPGEAGAPGLVGPRGERGFPGERGSPGAQGLQGPRGLPGTPGTDGPKGASGPAGPPGAQGPPGLQGMPGERGAAGIAGPKGDRGDVGEKGPEGAPGKDGGRGLTGPIGPPGPAGANGEKGEVGPPGPAGSAGARGAPGERGETGPPGPAGFAGPPGADGQPGAKGEQGEAGQKGDAGAPGPQGPSGAPGPQGPTGVTGPKGARGAQGPPGATGFPGAAGRVGPPGSNGNPGPPGPPGPSGKDGPKGARGDSGPPGRAGEPGLQGPAGPPGEKGEPGDDGPSGAEGPPGPQGLAGQRGIVGLPGQRGERGFPGLPGPSGEPGKQGAPGASGDRGPPGPVGPPGLTGPAGEPGREGSPGADGPPGRDGAAGVKGDRGETGAVGAPGAPGPPGSPGPAGPTGKQGDRGEAGAQGPMGPSGPAGARGIQGPQGPRGDKGEAGEPGERGLKGHRGFTGLQGLPGPPGPSGDQGASGPAGPSGPRGPPGPVGPSGKDGANGIPGPIGPPGPRGRSGETGPAGPPGNPGPPGPPGPPGPGIDMSAFAGLGPREKGPDPLQYMRADQAAGGLRQHDAE

SEQ ID NO: 5 - DNA, 3372 bp, Artificial Sequence
  In silico generated modification of human sequence
ggtgaatgttgtccagtttgtccagatggttctgaatctcctactgatcaagaaactaccggtgttgaaggtcccaaaggagatactggtcccagaggaccaaggggtccagccggacctcccggtagagatggaataccaggtcaaccaggtttgccaggtccgccaggtccaccaggtcctcccggtcctcccggacttggtggtaattttgctccacaattgtcttatggttatgatgaaaaatctacaggtggaatttctgtaccaggtccaatgggaccttctggacctcgtggtttgcctggaccaccaggcgccccaggtccacagggctttcaaggcccaccaggagagccaggagaacctggagcttcaggtcctatgggtccaaggggacctcccggacctcctggcaaaaatggtgatgatggagaagcaggtaaaccaggtagaccaggtgagcgtggcccaccaggaccacagggtgctagaggtttaccaggcactgctggtttgccaggcatgaaaggtcataggggtttttctggtttggatggcgcaaaaggagatgcaggccccgcaggtcccaaaggtgaaccaggcagtccaggtgaaaatggagctcccggtcaaatgggcccaaggggtttgccaggagagagaggcaggccaggtgctcccggcccagctggagctcgtggtaatgatggtgcaactggtgctgctggaccgcccggtccaactggacccgccggaccacctggttttccaggagctgttggtgctaaaggtgaagcaggccctcagggacctaggggctctgaaggtccacaaggtgtaagaggcgagccaggtccacctggacctgcgggtgctgctggcccagccggcaatccaggcgcagatggacaacccggtgcaaaaggtgctaacggtgctccaggaattgctggtgcaccaggtttcccaggagctagaggtccttccggaccccaaggtccaggtggtccacctggtcccaaaggtaattctggagaaccaggtgcccctggtagtaagggtgatacaggagcaaaaggagaaccaggaccagttggtgttcaaggaccgccaggaccagcaggagaagaaggtaaaagaggtgctagaggtgaacctggtccaactggtcttccaggaccacctggcgaaagaggtggtccaggttctagaggctttcccggagctgatggtgttgctggacctaaaggtccagcaggcgaaaggggttcaccaggaccagctggcccaaaaggttccccaggtgaagctggtagaccaggcgaagctggattgcctggtgccaaaggtttgactggttcaccaggctctcctggaccagatggtaaaacaggcccacctggccccgctggacaagatggcagacctggacctcctggtccacctggggccagaggacaagctggtgttatgggttttccaggtccaaaaggagctgctggcgaaccaggaaaagctggtgaacgtggagttcctggtccgccaggagcagtaggtccagccggtaaagatggtgaggccggtgctcaaggaccaccaggtccagctggtcccgcaggagagagaggagaacaaggacccgctggatctcctggttttcaaggcttgcctggtccagcaggaccgcctggcgaagctggaaaaccaggcgaacaaggtgttccaggtgatttgggagcaccaggtccaagtggtgctagaggagaaagaggttttcccggcgaacgtggtgtacagggtccacctggcccagcaggtcctcgtggtgctaatggtgctcccggtaatgatggagctaagggagacgccggcgcaccaggagcaccaggatcacaaggagctcctggattgcaaggtatgccaggtgagagaggtgctgctggtttaccaggtccgaaaggtgatagaggtgatgctggtcctaaaggagctgatggctctcctggaaaagacggtgttagaggtttgactggtccaatcggcccacctggtccagccggggccccaggagataaaggtgaatctggtccaagcggtccagcgggccctactggtgctaggggtgctcctggagataggggtgagcctggtcctcccggaccagcgggcttcgccggtccaccaggagcagatggtcaaccaggtgcaaagggtgaaccaggcgatgctggtgctaaaggagacgctggaccacccggacccgcaggacctgccggacctccggggccaattggaaatgttggagctccaggtgcaaaaggagccagaggttctgctggaccacctggtgcaactggttttcctggagcagctggaagggtcggtccgcctggtccctcgggaaatgctggtcctcctggtccgcctggacctgccggcaaagaaggtggtaaaggtcctaggggtgaaactggcccagctggtagacctggtgaagttggtccaccaggccctcctgggccggcaggagaaaaaggttctcctggtgccgatggtccagctggtgcacccggtactcctggtcctcagggaatagctggtcaaagaggtgttgttggtctaccaggacaaagaggtgaaaggggattcccaggacttccaggcccatcaggtgaacccggaaagcaaggtccatcaggtgcttctggagagagaggcccacccgggccaatgggtccaccaggattggcaggtcctcctggtgagtctggtagagaaggtgctccgggtgctgaaggttccccgggtagagatggttcaccaggtgctaagggagatagaggtgaaacaggaccagccggtcctcctggagctccaggcgcacccggggctccaggtccagtgggacccgctggaaaatctggtgatagaggagagactggacctgccggtcccgcagggccggtaggccccgttggtgcacgtggtcccgctggtccacagggccctagaggtgataaaggtgagactggtgaacaaggcgatagaggaattaaaggtcatagaggtttctctggtttgcaaggaccacctggtcctcctggatcaccaggtgaacaaggaccatctggtgctagtggaccagccggacctagaggtcctcccggatctgctggtgcccctggaaaagatggtttaaacggacttccaggacctattggtccaccaggaccaaggggcagaactggtgatgcaggacctgttggaccacctggaccgccaggtcctcctggcccaccgggtccaccttctgcaggctttgatttttcatttttgccacaaccaccacaagaaaaagctcatgatggt

SEQ ID NO: 6 - protein, 1124 aa, Artificial Sequence
  In silico generated modification of human sequence
GECCPVCPDGSESPTDQETTGVEGPKGDTGPRGPRGPAGPPGRDGIPGQPGLPGPPGPPGPPGPPGLGGNFAPQLSYGYDEKSTGGISVPGPMGPSGPRGLPGPPGAPGPQGFQGPPGEPGEPGASGPMGPRGPPGPPGKNGDDGEAGKPGRPGERGPPGPQGARGLPGTAGLPGMKGHRGFSGLDGAKGDAGPAGPKGEPGSPGENGAPGQMGPRGLPGERGRPGAPGPAGARGNDGATGAAGPPGPTGPAGPPGFPGAVGAKGEAGPQGPRGSEGPQGVRGEPGPPGPAGAAGPAGNPGADGQPGAKGANGAPGIAGAPGFPGARGPSGPQGPGGPPGPKGNSGEPGAPGSKGDTGAKGEPGPVGVQGPPGPAGEEGKRGARGEPGPTGLPGPPGERGGPGSRGFPGADGVAGPKGPAGERGSPGPAGPKGSPGEAGRPGEAGLPGAKGLTGSPGSPGPDGKTGPPGPAGQDGRPGPPGPPGARGQAGVMGFPGPKGAAGEPGKAGERGVPGPPGAVGPAGKDGEAGAQGPPGPAGPAGERGEQGPAGSPGFQGLPGPAGPPGEAGKPGEQGVPGDLGAPGPSGARGERGFPGERGVQGPPGPAGPRGANGAPGNDGAKGDAGAPGAPGSQGAPGLQGMPGERGAAGLPGPKGDRGDAGPKGADGSPGKDGVRGLTGPIGPPGPAGAPGDKGESGPSGPAGPTGARGAPGDRGEPGPPGPAGFAGPPGADGQPGAKGEPGDAGAKGDAGPPGPAGPAGPPGPIGNVGAPGAKGARGSAGPPGATGFPGAAGRVGPPGPSGNAGPPGPPGPAGKEGGKGPRGETGPAGRPGEVGPPGPPGPAGEKGSPGADGPAGAPGTPGPQGIAGQRGVVGLPGQRGERGFPGLPGPSGEPGKQGPSGASGERGPPGPMGPPGLAGPPGESGREGAPGAEGSPGRDGSPGAKGDRGETGPAGPPGAPGAPGAPGPVGPAGKSGDRGETGPAGPAGPVGPVGARGPAGPQGPRGDKGETGEQGDRGIKGHRGFSGLQGPPGPPGSPGEQGPSGASGPAGPRGPPGSAGAPGKDGLNGLPGPIGPPGPRGRTGDAGPVGPPGPPGPPGPPGPPSAGFDFSFLPQPPQEKAHDG

SEQ ID NO: 7 - DNA, 3207 bp, Artificial Sequence
  In silico generated modification of human sequence
ccccagaactacagcccgcaatacgattcttacgacgtcaaatctggtgttgctgttggtgggctagctggttatccaggtcccgctggtcctcccggaccgcctggtccaccaggtacatctggacatcctggctcacccggttcaccaggttatcaaggtccacctggtgagcccggacaagctggtccaagtggtcctcctggtccgccaggagctatcggtccatctggtccagccggtaaggatggtgaatctggtagaccaggcagacctggcgaaaggggcttgccaggtccacctggaataaaaggccctgcgggaataccaggttttcctggcatgaaaggtcacagaggttttgatggtagaaatggcgaaaagggtgaaactggtgctcctggtttaaaaggagaaaatggcttgccaggcgaaaatggagctccaggcccaatgggtccaaggggtgctccaggagagaggggtagacctggtttgccaggagcagccggtgcccgtggtaatgatggtgctagaggatctgacggacaacctggcccacctggaccacctggaacagctggctttcctggcagtcccggcgccaaaggtgaagttggtccagcaggttcacccggttcaaatggtgctcctggacaaaggggagaacccggacctcagggtcatgctggtgctcaaggtcctcccggtccacccggaattaacggtagcccaggaggtaaaggcgaaatgggtcctgccggaatacctggtgctccaggcttaatgggtgcgagaggccctcctggtccagctggagctaacggtgctccaggtctaagaggtggagccggtgaaccaggaaagaatggtgctaaaggcgaacccggaccaaggggagaacgtggagaatgtggcatacccggagtacccggtgctaaaggtgaggatggcaaagatggttcccctggtgagcctggggctaatggattgcctggtgctgcaggcgaaagaggagctcctggattcagaggacccgcaggaccaaatggaataccaggtgaaaaaggcccagcaggtgaaagaggtgcgcccggaccggccggaccacgtggagcagcaggagaacccggcagagatggagttccaggaggtccaggaatgagaggtatgccaggttcgccaggtggtcctggatctgatggtaaacccggtccaccgggaagccaaggtgagtcaggcagacccggtcctcctggcccctcaggaccaaggggtcaaccaggagttatgggctttcctggcccaaaaggcaacgatggcgcacctggtaaaaatggtgaaagaggcggacccggtggtcccggcccacaaggacctcctggaaagaatggtgaaacaggaccacaagggccacccggcccaacaggtccaggtggagacaaaggtgataccggaccacctggaccccaaggattgcaaggactacctggtacaggaggacctcccggagagaatggtaaaccaggtgaacctggtccaaaaggtgatgctggagctccaggagctccgggtggaaaaggtgatgcaggcgcgccaggtgaacgtggtccaccaggtttggcaggagcaccaggattgagaggaggagctggtccacctggtcccgaaggaggtaaaggagctgcaggaccacctggtcctcctggtgctgccgggactccaggtttgcaaggtatgccaggagagagaggcggtttgggttcacccggaccaaaaggtgataagggtgaacctggttgtcctggcgcagacggtgttcccggtaaagatggtcccagaggaccaactggaccaattggacctcccggacccgcaggtcagcctggtgataaaggtgaaggtggtgcacccggattaccaggaattgctggcccaaggggtagtccaggtgaaaggggagaaacaggtccaccaggcccagctggattcccaggtgcccctggacaaaatggtgagcctggtggtaaaggtgaaagaggggctccaggcgaaaaaggagagggtggaccaccaggagttgcaggtccaccaggaggttccggaccagcaggacctccgggtccccagggtgttaaaggagaaagaggttctcctggaggtccaggagccgctggttttccaggagctaggggattgccaggaccgcccggatctaatggtaatcctggaccacccggtccttctggatctcctggtaaagacggtccacctggaccggcgggaaatactggtgcaccgggttcacccggtgtatctggaccaaagggagatgctggtcaacccggtgaaaaaggttcaccaggagctcagggtccgcccggcgcaccaggcccgttgggaatagctggaattactggtgctaggggcttggctggaccgccaggcatgccaggtcctaggggcagtccaggtccacaaggcgttaaaggcgagtcaggaaaaccaggtgctaatggtttatcgggagaaagaggtccgcctgggcctcagggtttgcctggattggctggtactgctggtgagccaggtagagatggtaatcccggttctgatggattacccggcagggacggatcccctggtggcaagggagatagaggtgaaaatggttccccaggtgctcccggtgctccagggcatccgggacctcccggtcctgtaggaccagccggcaaatctggtgatagaggagagtcaggtcctgcaggtcccgcaggagctcctggccccgccggatctaggggtgcaccaggtccccaaggccctagaggtgacaagggtgaaacaggtgagagaggtgctgcgggcattaaaggtcatagaggctttccaggtaatcctggtgcacctggtagtcctggaccagctggtcaacaaggtgctattggtagtcctggccctgcaggtcctcgtggaccagttggaccaagcggaccacctggtaaagatggaacttctggtcatccaggtccaataggtcctcctggaccaaggggcaatagaggagaaagaggctctgaaggtagccctggtcatccaggacagccaggacctcctggaccaccaggtgctccggggccctgttgtggtggtgttggtgctgctgctattgctggcatcggcggtgaaaaagctggtggattc

SEQ ID NO: 8 - protein, 1069 aa, Artificial Sequence
  In silico generated modification of human sequence
PQNYSPQYDSYDVKSGVAVGGLAGYPGPAGPPGPPGPPGTSGHPGSPGSPGYQGPPGEPGQAGPSGPPGPPGAIGPSGPAGKDGESGRPGRPGERGLPGPPGIKGPAGIPGFPGMKGHRGFDGRNGEKGETGAPGLKGENGLPGENGAPGPMGPRGAPGERGRPGLPGAAGARGNDGARGSDGQPGPPGPPGTAGFPGSPGAKGEVGPAGSPGSNGAPGQRGEPGPQGHAGAQGPPGPPGINGSPGGKGEMGPAGIPGAPGLMGARGPPGPAGANGAPGLRGGAGEPGKNGAKGEPGPRGERGECGIPGVPGAKGEDGKDGSPGEPGANGLPGAAGERGAPGFRGPAGPNGIPGEKGPAGERGAPGPAGPRGAAGEPGRDGVPGGPGMRGMPGSPGGPGSDGKPGPPGSQGESGRPGPPGPSGPRGQPGVMGFPGPKGNDGAPGKNGERGGPGGPGPQGPPGKNGETGPQGPPGPTGPGGDKGDTGPPGPQGLQGLPGTGGPPGENGKPGEPGPKGDAGAPGAPGGKGDAGAPGERGPPGLAGAPGLRGGAGPPGPEGGKGAAGPPGPPGAAGTPGLQGMPGERGGLGSPGPKGDKGEPGCPGADGVPGKDGPRGPTGPIGPPGPAGQPGDKGEGGAPGLPGIAGPRGSPGERGETGPPGPAGFPGAPGQNGEPGGKGERGAPGEKGEGGPPGVAGPPGGSGPAGPPGPQGVKGERGSPGGPGAAGFPGARGLPGPPGSNGNPGPPGPSGSPGKDGPPGPAGNTGAPGSPGVSGPKGDAGQPGEKGSPGAQGPPGAPGPLGIAGITGARGLAGPPGMPGPRGSPGPQGVKGESGKPGANGLSGERGPPGPQGLPGLAGTAGEPGRDGNPGSDGLPGRDGSPGGKGDRGENGSPGAPGAPGHPGPPGPVGPAGKSGDRGESGPAGPAGAPGPAGSRGAPGPQGPRGDKGETGERGAAGIKGHRGFPGNPGAPGSPGPAGQQGAIGSPGPAGPRGPVGPSGPPGKDGTSGHPGPIGPPGPRGNRGERGSEGSPGHPGQPGPPGPPGAPGPCCGGVGAAAIAGIGGEKAGGF

SEQ ID NO: 9 - DNA, 3207 bp, Artificial Sequence
  In silico generated modification of human sequence
ccccagaactacagcccgcaatacgattcttacgacgtcaaatctggtgttgctgttggtgggctagctggttatccaggtcccgctggtcctcccggaccgcctggtccaccaggtacatctggacatcctggctcacccggttcaccaggttatcaaggtccacctggtgagcccggacaagctggtccaagtggtcctcctggtccgccaggagctatcggtccatctggtccagccggtaaggatggtgaatctggtagaccagggagtccaggcggcaaaggcttgccaggtccacctggaataaaaggccctgcgggaataccaggttttcctggcatgaaaggtcacagaggttttgatggtagaaatggcgaaaagggtgaaactggtgctcctggatcgccaggtggtaagggttcgccaggaggaaagggagctccaggcccaatgggtccaaggggttcaccaggcggtaaaggtagacctggtttgccaggagcagccggtgcccgtggtaatgatggtgctagaggatctgacggacaacctggcccacctggaccacctggaacagctggctttcctggcagtcccggcgccaaaggtgaagttggtccagcaggttcacccggttcaaatggtgctcctggacaaaggggagaacccggacctcagggtcatgctggtgctcaaggtcctcccggtccacccggaattaacggtagcccaggaggtaaaggcgaaatgggtcctgccggaatacctggtgctccaggcttaatgggtgcgagaggccctcctggtccagctggagctaacggtgctccaggtctaagaggtggagccggtgaaccaggaaagaatggtgctaaaggcgaacccggaccaaggggagaacgtggagaagccggcatacccggagtacccggtgctaaaggtgaggatggcaaagatggttcccctggtgagcctggggctaatggattgcctggtgctgcaggcgaaagaggagctcctggattcagaggacccgcaggaccaaatggaataccaggtgaaaaaggcccagcaggtgaaagaggtgcgcccggaccggccggaccacgtggagcagcaggagaacccggcagagatggagttccaggaggtccaggaatgagaggtatgccaggttcgccaggtggtcctggatctgatggtaaacccggtccaccgggaagccaaggtgagtcaggcagacccggtcctcctggcccctcaggaccaaggggtcaaccaggagttatgggctttcctggcccaaaaggcaacgatggcgcacctggtaaaaatggtgaaagaggcggacccggtggtcccggcccacaaggacctcctggaaagaatggtgaaacaggaccacaagggccacccggcccaacaggtccaggtggagacaaaggtgataccggaccacctggaccccaaggattgcaaggactacctggtacaggaggacctcccggagagaatggtaaaccaggtgaacctggtccaaaaggtgatgctggagctccaggagctccgggtggaaaaggtgatgcaggtagtcctggtggaaaaggtccaccaggtttggcaggagcaccaggattgagaggaggagctggtccacctggtcccgaaggaggtaaaggagctgcaggaccacctggtcctcctggtgctgccgggactccaggtttgcaaggttcgcctggtggcaaaggcggtttgggttcacccggaccaaaaggtgataagggtgaacctggtggtcctggcgcagacggtgttcccggtaaagatggtcccagaggaccaactggaccaattggacctcccggacccgcaggtcagcctggtgataaaggtgaaggtggtgcacccggattaccaggaattgctggcccaaggggtagtccaggtgaaaggggagaaacaggtccaccaggcccagctggattcccaggtgcccctggacaaaatggtgagcctggtggtaaaggtgaaagaggggctccaggcgaaaaaggagagggtggaccaccaggagttgcaggtccaccaggaggttccggaccagcaggacctccgggtccccagggtgttaaaggagaaagaggttctcctggaggtccaggagccgctggttttccaggagctaggggattgccaggaccgcccggatctaatggtaatcctggaccacccggtccttctggatctcctggtaaagacggtccacctggaccggcgggaaatactggtgcaccgggttcacccggtgtatctggaccaaagggagatgctggtcaacccggtgaaaaaggttcaccaggagctcagggtccgcccggcgcaccaggcccgttgggaatagctggaattactggtgctaggggcttggctggaccgccaggcatgccaggtcctaggggcagtccaggtccacaaggcgttaaaggcgagtcaggaaaaccaggtgctaatggtttatcgggagaaagaggtccgcctgggcctcagggtttgcctggattggctggtactgctggtgagccaggtagagatggtaatcccggttctgatggattacccggcagggacggatcccctggtggcaagggagatagaggtgaaaatggttccccaggtgctcccggtgctccagggcatccgggacctcccggtcctgtaggaccagccggcaaatctggtgatagaggagagtcaggtcctgcaggtcccgcaggagctcctggccccgccggatctaggggtgcaccaggtccccaaggccctagaggtgacaagggtgaaacaggtgagagaggtgctgcgggcattaaaggtcatagaggctttccaggtaatcctggtgcacctggtagtcctggaccagctggtcaacaaggtgctattggtagtcctggccctgcaggtcctcgtggaccagttggaccaagcggaccacctggtaaagatggaacttctggtcatccaggtccaataggtcctcctggaccaaggggcaatagaggagaaagaggctctgaaggtagccctggtcatccaggacagccaggacctcctggaccaccaggtgctccggggccctgttgtggtggtgttggtgctgctgctattgctggcatcggcggtgaaaaagctggtggattc

SEQ ID NO: 10 - protein, 1069 aa, Artificial Sequence
  In silico generated modification of human sequence
PQNYSPQYDSYDVKSGVAVGGLAGYPGPAGPPGPPGPPGTSGHPGSPGSPGYQGPPGEPGQAGPSGPPGPPGAIGPSGPAGKDGESGRPGSPGGKGLPGPPGIKGPAGIPGFPGMKGHRGFDGRNGEKGETGAPGSPGGKGSPGGKGAPGPMGPRGSPGGKGRPGLPGAAGARGNDGARGSDGQPGPPGPPGTAGFPGSPGAKGEVGPAGSPGSNGAPGQRGEPGPQGHAGAQGPPGPPGINGSPGGKGEMGPAGIPGAPGLMGARGPPGPAGANGAPGLRGGAGEPGKNGAKGEPGPRGERGEAGIPGVPGAKGEDGKDGSPGEPGANGLPGAAGERGAPGFRGPAGPNGIPGEKGPAGERGAPGPAGPRGAAGEPGRDGVPGGPGMRGMPGSPGGPGSDGKPGPPGSQGESGRPGPPGPSGPRGQPGVMGFPGPKGNDGAPGKNGERGGPGGPGPQGPPGKNGETGPQGPPGPTGPGGDKGDTGPPGPQGLQGLPGTGGPPGENGKPGEPGPKGDAGAPGAPGGKGDAGSPGGKGPPGLAGAPGLRGGAGPPGPEGGKGAAGPPGPPGAAGTPGLQGSPGGKGGLGSPGPKGDKGEPGGPGADGVPGKDGPRGPTGPIGPPGPAGQPGDKGEGGAPGLPGIAGPRGSPGERGETGPPGPAGFPGAPGQNGEPGGKGERGAPGEKGEGGPPGVAGPPGGSGPAGPPGPQGVKGERGSPGGPGAAGFPGARGLPGPPGSNGNPGPPGPSGSPGKDGPPGPAGNTGAPGSPGVSGPKGDAGQPGEKGSPGAQGPPGAPGPLGIAGITGARGLAGPPGMPGPRGSPGPQGVKGESGKPGANGLSGERGPPGPQGLPGLAGTAGEPGRDGNPGSDGLPGRDGSPGGKGDRGENGSPGAPGAPGHPGPPGPVGPAGKSGDRGESGPAGPAGAPGPAGSRGAPGPQGPRGDKGETGERGAAGIKGHRGFPGNPGAPGSPGPAGQQGAIGSPGPAGPRGPVGPSGPPGKDGTSGHPGPIGPPGPRGNRGERGSEGSPGHPGQPGPPGPPGAPGPCCGGVGAAAIAGIGGEKAGGF

SEQ ID NO: 11 - DNA, 3207 bp, Artificial Sequence
  In silico generated modification of human sequence
ccccagaactacagcccgcaatacgattcttacgacgtcaaatctggtgttgctgttggtgggctagctggttatccaggtcccgctggtcctcccggaccgcctggtccaccaggtacatctggacatcctggctcacccggttcaccaggttatcaaggtccacctggtgagcccggacaagctggtccaagtggtcctcctggtccgccaggagctatcggtccatctggtccagccggtaaggatggtgaatctggcgaaagaggctttcctggtgaaagaggtgttcagggtccacctggaataaaaggccctgcgggaataccaggttttcctggcatgaaaggtcacagaggttttgatggtagaaatggcgaaaagggtgaaactggtgctcctggatcgccaggtggtaagggttcgccaggaggaaagggagctccaggcccaatgggtccaaggggttcaccaggcggtaaaggtagacctggtttgccaggagcagccggtgcccgtggtaatgatggtgctagaggatctgacggacaacctggcccacctggaccacctggaacagctggctttcctggcagtcccggcgccaaaggtgaagttggtccagcaggttcacccggttcaaatggtgctcctggacaaaggggagaacccggacctcagggtcatgctggtgctcaaggtcctcccggtccacccggaattaacggtagcccaggaggtaaaggcgaaatgggtcctgccggaatacctggtgctccaggcttaatgggtgcgagaggccctcctggtccagctggagctaacggtgctccaggtctaagaggtggagccggtgaaccaggaaagaatggtgctaaaggcgaacccggaccaaggggagaacgtggagaagccggcatacccggagtacccggtgctaaaggtgaggatggcaaagatggttcccctggtgagcctggggctaatggattgcctggtgctgcaggcgaaagaggagctcctggattcagaggacccgcaggaccaaatggaataccaggtgaaaaaggcccagcaggtgaaagaggtgcgcccggaccggccggaccacgtggagcagcaggagaacccggcagagatggagttccaggaggtccaggaatgagaggtatgccaggttcgccaggtggtcctggatctgatggtaaacccggtccaccgggaagccaaggtgagtcaggcagacccggtcctcctggcccctcaggaccaaggggtcaaccaggagttatgggctttcctggcccaaaaggcaacgatggcgcacctggtaaaaatggtgaaagaggcggacccggtggtcccggcccacaaggacctcctggaaagaatggtgaaacaggaccacaagggccacccggcccaacaggtccaggtggagacaaaggtgataccggaccacctggaccccaaggattgcaaggactacctggtacaggaggacctcccggagagaatggtaaaccaggtgaacctggtccaaaaggtgatgctggagctccaggagctccgggtggaaaaggtgatgcaggtagtcctggtggaaaaggtccaccaggtttggcaggagcaccaggattgagaggaggagctggtccacctggtcccgaaggaggtaaaggagctgcaggaccacctggtcctcctggtgctgccgggactccaggtttgcaaggttcgcctggtggcaaaggcggtttgggttcacccggaccaaaaggtgataagggtgaacctggtggtcctggcgcagacggtgttcccggtaaagatggtcccagaggaccaactggaccaattggacctcccggacccgcaggtcagcctggtgataaaggtgaaggtggtgcacccggattaccaggaattgctggcccaaggggtagtccaggtgaaaggggagaaacaggtccaccaggcccagctggattcccaggtgcccctggacaaaatggtgagcctggtggtaaaggtgaaagaggggctccaggcgaaaaaggagagggtggaccaccaggagttgcaggtccaccaggaggttccggaccagcaggacctccgggtccccagggtgttaaaggagaaagaggttctcctggaggtccaggagccgctggttttccaggagctaggggattgccaggaccgcccggatctaatggtaatcctggaccacccggtccttctggatctcctggtaaagacggtccacctggaccggcgggaaatactggtgcaccgggttcacccggtgtatctggaccaaagggagatgctggtcaacccggtgaaaaaggttcaccaggagctcagggtccgcccggcgcaccaggcccgttgggaatagctggaattactggtgctaggggcttggctggaccgccaggcatgccaggtcctaggggcagtccaggtccacaaggcgttaaaggcgagtcaggaaaaccaggtgctaatggtttatcgggagaaagaggtccgcctgggcctcagggtttgcctggattggctggtactgctggtgagccaggtagagatggtaatcccggttctgatggattacccggcagggacggatcccctggtggcaagggagatagaggtgaaaatggttccccaggtgctcccggtgctccagggcatccgggacctcccggtcctgtaggaccagccggcaaatctggtgatagaggagagtcaggtcctgcaggtcccgcaggagctcctggccccgccggatctaggggtgcaccaggtccccaaggccctagaggtgacaagggtgaaacaggtgagagaggtgctgcgggcattaaaggtcatagaggctttccaggtaatcctggtgcacctggtagtcctggaccagctggtcaacaaggtgctattggtagtcctggccctgcaggtcctcgtggaccagttggaccaagcggaccacctggtaaagatggaacttctggtcatccaggtccaataggtcctcctggaccaaggggcaatagaggagaaagaggctctgaaggtagccctggtcatccaggacagccaggacctcctggaccaccaggtgctccggggccctgttgtggtggtgttggtgctgctgctattgctggcatcggcggtgaaaaagctggtggattc

SEQ ID NO: 12 - protein, 1069 aa, Artificial Sequence
  In silico generated modification of human sequence
PQNYSPQYDSYDVKSGVAVGGLAGYPGPAGPPGPPGPPGTSGHPGSPGSPGYQGPPGEPGQAGPSGPPGPPGAIGPSGPAGKDGESGERGFPGERGVQGPPGIKGPAGIPGFPGMKGHRGFDGRNGEKGETGAPGSPGGKGSPGGKGAPGPMGPRGSPGGKGRPGLPGAAGARGNDGARGSDGQPGPPGPPGTAGFPGSPGAKGEVGPAGSPGSNGAPGQRGEPGPQGHAGAQGPPGPPGINGSPGGKGEMGPAGIPGAPGLMGARGPPGPAGANGAPGLRGGAGEPGKNGAKGEPGPRGERGEAGIPGVPGAKGEDGKDGSPGEPGANGLPGAAGERGAPGFRGPAGPNGIPGEKGPAGERGAPGPAGPRGAAGEPGRDGVPGGPGMRGMPGSPGGPGSDGKPGPPGSQGESGRPGPPGPSGPRGQPGVMGFPGPKGNDGAPGKNGERGGPGGPGPQGPPGKNGETGPQGPPGPTGPGGDKGDTGPPGPQGLQGLPGTGGPPGENGKPGEPGPKGDAGAPGAPGGKGDAGSPGGKGPPGLAGAPGLRGGAGPPGPEGGKGAAGPPGPPGAAGTPGLQGSPGGKGGLGSPGPKGDKGEPGGPGADGVPGKDGPRGPTGPIGPPGPAGQPGDKGEGGAPGLPGIAGPRGSPGERGETGPPGPAGFPGAPGQNGEPGGKGERGAPGEKGEGGPPGVAGPPGGSGPAGPPGPQGVKGERGSPGGPGAAGFPGARGLPGPPGSNGNPGPPGPSGSPGKDGPPGPAGNTGAPGSPGVSGPKGDAGQPGEKGSPGAQGPPGAPGPLGIAGITGARGLAGPPGMPGPRGSPGPQGVKGESGKPGANGLSGERGPPGPQGLPGLAGTAGEPGRDGNPGSDGLPGRDGSPGGKGDRGENGSPGAPGAPGHPGPPGPVGPAGKSGDRGESGPAGPAGAPGPAGSRGAPGPQGPRGDKGETGERGAAGIKGHRGFPGNPGAPGSPGPAGQQGAIGSPGPAGPRGPVGPSGPPGKDGTSGHPGPIGPPGPRGNRGERGSEGSPGHPGQPGPPGPPGAPGPCCGGVGAAAIAGIGGEKAGGF

SEQ ID NO: 13 - DNA, 510 bp, Artificial Sequence
  In silico generated modification of human sequence
atgatgtctttcgttcaaaaggggagctggttgttgttggctctactccacccgaccattattttggcccagcaggaagctgttgaaggtggatgttctcatttgggtcaaagctacgcggatagagatgtttggaagccagaaccatgtcaaatctgtgtctgtgatagcggttcggtcctgtgtgatgacattatttgtgacgatcaggagctcgactgtcctaaccctgagatcccattcggtgagtgttgtgctgtttgtccgcaaccgcccactgctccaactcgtcctcccaatggtcaaggtccccaaggtccaaaaggtgatccggggccaccaggcataccaggtagaaatggtgacccaggaatacccggacagccaggttccccagggagtccaggacctcccggaatttgtgagtcttgtccaactggaccccagaactacagcccgcaatacgattcttacgacgtcaaatctggtgttgctgttggtgggctagct

SEQ ID NO: 14 - protein, 170 aa, Artificial Sequence
  In silico generated modification of human sequence
MMSFVQKGSWLLLALLHPTIILAQQEAVEGGCSHLGQSYADRDVWKPEPCQICVCDSGSVLCDDIICDDQELDCPNPEIPFGECCAVCPQPPTAPTRPPNGQGPQGPKGDPGPPGIPGRNGDPGIPGQPGSPGSPGPPGICESCPTGPQNYSPQYDSYDVKSGVAVGGLA

SEQ ID NO: 15 - DNA, 3207 bp, Artificial Sequence
  In silico generated modification of human sequence
ccccagaactacagcccgcaatacgattcttacgacgtcaaatctggtgttgctgttggtgggctagctggttatccaggtcccgctggtcctcccggaccgcctggtccaccaggtacatctggacatcctggctcacccggttcaccaggttatcaaggtccacctggtgagcccggacaagctggtccaagtggtcctcctggtccgccaggagctatcggtccatctggtccagccggtaaggatggtgaatctggtagaccaggcagacctggcgaaaggggcttgccaggtccacctggaataaaaggccctgcgggaataccaggttttcctggcatgaaaggtcacagaggttttgatggtagaaatggcgaaaagggtgaaactggtgctcctggtttaaaaggagaaaatggcttgccaggcgaaaatggagctccaggcccaatgggtccaaggggtgctccaggagagaggggtagacctggtttgccaggagcagccggtgcccgtggtaatgatggtgctagaggatctgacggacaacctggcccacctggaccacctggaacagctggctttcctggcagtcccggcgccaaaggtgaagttggtccagcaggttcacccggttcaaatggtgctcctggacaaaggggagaacccggacctcagggtcatgctggtgctcaaggtcctcccggtccacccggaattaacggtagcccaggaggtaaaggcgaaatgggtcctgccggaatacctggtgctccaggcttaatgggtgcgagaggccctcctggtccagctggagctaacggtgctccaggtctaagaggtggagccggtgaaccaggaaagaatggtgctaaaggcgaacccggaccaaggggagaacgtggagaagccggcatacccggagtacccggtgctaaaggtgaggatggcaaagatggttcccctggtgagcctggggctaatggattgcctggtgctgcaggcgaaagaggagctcctggattcagaggacccgcaggaccaaatggaataccaggtgaaaaaggcccagcaggtgaaagaggtgcgcccggaccggccggaccacgtggagcagcaggagaacccggcagagatggagttccaggaggtccaggaatgagaggtatgccaggttcgccaggtggtcctggatctgatggtaaacccggtccaccgggaagccaaggtgagtcaggcagacccggtcctcctggcccctcaggaccaaggggtcaaccaggagttatgggctttcctggcccaaaaggcaacgatggcgcacctggtaaaaatggtgaaagaggcggacccggtggtcccggcccacaaggacctcctggaaagaatggtgaaacaggaccacaagggccacccggcccaacaggtccaggtggagacaaaggtgataccggaccacctggaccccaaggattgcaaggactacctggtacaggaggacctcccggagagaatggtaaaccaggtgaacctggtccaaaaggtgatgctggagctccaggagctccgggtggaaaaggtgatgcaggcgcgccaggtgaacgtggtccaccaggtttggcaggagcaccaggattgagaggaggagctggtccacctggtcccgaaggaggtaaaggagctgcaggaccacctggtcctcctggtgctgccgggactccaggtttgcaaggtatgccaggagagagaggcggtttgggttcacccggaccaaaaggtgataagggtgaacctggtggtcctggcgcagacggtgttcccggtaaagatggtcccagaggaccaactggaccaattggacctcccggacccgcaggtcagcctggtgataaaggtgaaggtggtgcacccggattaccaggaattgctggcccaaggggtagtccaggtgaaaggggagaaacaggtccaccaggcccagctggattcccaggtgcccctggacaaaatggtgagcctggtggtaaaggtgaaagaggggctccaggcgaaaaaggagagggtggaccaccaggagttgcaggtccaccaggaggttccggaccagcaggacctccgggtccccagggtgttaaaggagaaagaggttctcctggaggtccaggagccgctggttttccaggagctaggggattgccaggaccgcccggatctaatggtaatcctggaccacccggtccttctggatctcctggtaaagacggtccacctggaccggcgggaaatactggtgcaccgggttcacccggtgtatctggaccaaagggagatgctggtcaacccggtgaaaaaggttcaccaggagctcagggtccgcccggcgcaccaggcccgttgggaatagctggaattactggtgctaggggcttggctggaccgccaggcatgccaggtcctaggggcagtccaggtccacaaggcgttaaaggcgagtcaggaaaaccaggtgctaatggtttatcgggagaaagaggtccgcctgggcctcagggtttgcctggattggctggtactgctggtgagccaggtagagatggtaatcccggttctgatggattacccggcagggacggatcccctggtggcaagggagatagaggtgaaaatggttccccaggtgctcccggtgctccagggcatccgggacctcccggtcctgtaggaccagccggcaaatctggtgatagaggagagtcaggtcctgcaggtcccgcaggagctcctggccccgccggatctaggggtgcaccaggtccccaaggccctagaggtgacaagggtgaaacaggtgagagaggtgctgcgggcattaaaggtcatagaggctttccaggtaatcctggtgcacctggtagtcctggaccagctggtcaacaaggtgctattggtagtcctggccctgcaggtcctcgtggaccagttggaccaagcggaccacctggtaaagatggaacttctggtcatccaggtccaataggtcctcctggaccaaggggcaatagaggagaaagaggctctgaaggtagccctggtcatccaggacagccaggacctcctggaccaccaggtgctccggggccctgttgtggtggtgttggtgctgctgctattgctggcatcggcggtgaaaaagctggtggattc

SEQ ID NO: 16 - protein, 1069 aa, Artificial Sequence
  In silico generated modification of human sequence
PQNYSPQYDSYDVKSGVAVGGLAGYPGPAGPPGPPGPPGTSGHPGSPGSPGYQGPPGEPGQAGPSGPPGPPGAIGPSGPAGKDGESGRPGRPGERGLPGPPGIKGPAGIPGFPGMKGHRGFDGRNGEKGETGAPGLKGENGLPGENGAPGPMGPRGAPGERGRPGLPGAAGARGNDGARGSDGQPGPPGPPGTAGFPGSPGAKGEVGPAGSPGSNGAPGQRGEPGPQGHAGAQGPPGPPGINGSPGGKGEMGPAGIPGAPGLMGARGPPGPAGANGAPGLRGGAGEPGKNGAKGEPGPRGERGEAGIPGVPGAKGEDGKDGSPGEPGANGLPGAAGERGAPGFRGPAGPNGIPGEKGPAGERGAPGPAGPRGAAGEPGRDGVPGGPGMRGMPGSPGGPGSDGKPGPPGSQGESGRPGPPGPSGPRGQPGVMGFPGPKGNDGAPGKNGERGGPGGPGPQGPPGKNGETGPQGPPGPTGPGGDKGDTGPPGPQGLQGLPGTGGPPGENGKPGEPGPKGDAGAPGAPGGKGDAGAPGERGPPGLAGAPGLRGGAGPPGPEGGKGAAGPPGPPGAAGTPGLQGMPGERGGLGSPGPKGDKGEPGGPGADGVPGKDGPRGPTGPIGPPGPAGQPGDKGEGGAPGLPGIAGPRGSPGERGETGPPGPAGFPGAPGQNGEPGGKGERGAPGEKGEGGPPGVAGPPGGSGPAGPPGPQGVKGERGSPGGPGAAGFPGARGLPGPPGSNGNPGPPGPSGSPGKDGPPGPAGNTGAPGSPGVSGPKGDAGQPGEKGSPGAQGPPGAPGPLGIAGITGARGLAGPPGMPGPRGSPGPQGVKGESGKPGANGLSGERGPPGPQGLPGLAGTAGEPGRDGNPGSDGLPGRDGSPGGKGDRGENGSPGAPGAPGHPGPPGPVGPAGKSGDRGESGPAGPAGAPGPAGSRGAPGPQGPRGDKGETGERGAAGIKGHRGFPGNPGAPGSPGPAGQQGAIGSPGPAGPRGPVGPSGPPGKDGTSGHPGPIGPPGPRGNRGERGSEGSPGHPGQPGPPGPPGAPGPCCGGVGAAAIAGIGGEKAGGF

SEQ ID NO: 17 - DNA, 819 bp, Artificial Sequence
  In silico generated modification of human sequence
gggccctgttgtggtggtgttggtgctgctgctattgctggcatcggcggtgaaaaagctggtggattcgctccatattatggtgatgagcccatggactttaagattaatacggatgagatcatgacgtccctgaaatcggtcaatgggcaaattgaatcgttgatttcacccgatggttccagaaagaatccagctaggaactgtcgtgacctaaaattctgtcaccccgaattaaaatccggtgaatattgggtcgatccaaatcaaggctgtaagttggacgctattaaggtgttctgtaacatggaaactggcgaaacgtgtatttctgcgaatcccctcaatgttcctagaaaacactggtggactgatagttctgctgagaagaagcatgtttggttcggtgaatccatggacggtggttttcaatttagctacggtaatccagaattgccagaggatgttttggacgttcaattggcattcttgcgtttgttgtcatcaagggcgtcgcagaatataacttatcattgtaagaactccatcgcctatatggatcaagcatccggcaacgtgaagaaggcattaaagttgatgggttctaatgagggcgaatttaaggctgaaggtaacagcaagttcacctatactgttctggaggatggttgtactaaacacacgggtgaatggtctaagacggtgttcgaatacagaacgaggaaggctgtgagattgcccattgttgacatagctccatacgatataggtggtccagatcaagaatttggggttgatgttggtccggtttgtttcttg

SEQ ID NO: 18 - protein, 273 aa, Artificial Sequence
  In silico generated modification of human sequence
GPCCGGVGAAAIAGIGGEKAGGFAPYYGDEPMDFKINTDEIMTSLKSVNGQIESLISPDGSRKNPARNCRDLKFCHPELKSGEYWVDPNQGCKLDAIKVFCNMETGETCISANPLNVPRKHWWTDSSAEKKHVWFGESMDGGFQFSYGNPELPEDVLDVQLAFLRLLSSRASQNITYHCKNSIAYMDQASGNVKKALKLMGSNEGEFKAEGNSKFTYTVLEDGCTKHTGEWSKTVFEYRTRKAVRLPIVDIAPYDIGGPDQEFGVDVGPVCFL

(19) United States
(12) Patent Application Publication
(10) Pub. No.: US 2009/0291474 A1
(43) Pub. Date: Nov. 26, 2009

(54) COLLAGEN-LIKE POLYPEPTIDES AND ENCODING POLYNUCLEOTIDES

(76) Inventors: Szu-Wen Wang, Irvine, CA (US); Nancy A. Da Silva, Irvine, CA (US); She-Pin Hung, Aliso Viejo, CA (US); Richard H. Lathrop, Irvine, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 12/467,184

(22) Filed: May 15, 2009

Related U.S. Application Data
(60) Provisional application No. 61/054,113, filed on May 16, 2008.

(51) Int. Cl. C12P 19/34; C07H 21/02; C12N 15/74; C07K 14/78; C07H 21/00

(52) U.S. Cl. 435/91.1; 536/23.1; 435/320.1; 530/350

(57) ABSTRACT

Provided herein are isolated polynucleotides encoding collagen-like polypeptide domains and the encoded polypeptides. In one such polypeptide, at least a portion of the encoded collagen-like polypeptide domain is in the form of Gly-X-Y triads, where X and Y symbolize individual amino acids; X is proline in at least 20% of the triads; Y is proline in at least 20% of the triads. The encoding polynucleotide of such a polypeptide encodes a region having at least 50 consecutive amino acids, wherein the region is at least 90% identical to an amino acid sequence of a naturally occurring collagen polypeptide; and no more than 98% of the nucleotides of the collagen-like polypeptide domain-encoding portion of the polynucleotide fall into a window of 100 consecutive nucleotides in which the nucleotides of the window have greater than 98% identity to a naturally occurring collagen-encoding nucleotide sequence.

This Application claims priority to U.S. Provisional Application No. 61/054,113, filed May 16, 2008, the entirety of which is hereby incorporated by reference.

### STATEMENT REGARDING FEDERALLY SPONSORED R&D

This project was sponsored in part by the U.S. Government, which has a paid-up license in this invention and the right in limited circumstances to require the patent owner to license others on reasonable terms as provided for by the terms of Grant No. DMR-0706669 awarded by the National Science Foundation.

### BACKGROUND

1. Technical Field

This application relates to synthetic collagen polypeptides, polynucleotides encoding the same, amino acid and nucleotide sequences thereof, and methods related thereto.

2. Description of the Related Art

Natural and biomimetic substrates, including collagen, can direct cellular behavior. Cells respond to specific biochemical and mechanical signals that exist within their extracellular matrices. These factors regulate important cellular behaviors, such as adhesion, proliferation, differentiation, and apoptosis. By introducing cell-interacting ligands onto surfaces, specific cellular behavior can be elicited. The diversity of materials and the control over cellular functions potentially comes from the combination of divergent properties, which include the concentrations and combinations of cell-interaction sites, spatial geometry of these sites, mechanical properties of the substrate material, and the material degradation kinetics.

Collagen, the most abundant protein in mammals, has been shown to be an integral component in influencing cellular behavior in the native extracellular matrix. It has been shown to dynamically control the life cycle and function of cells through natural binding sites; in turn, cells are able to remodel the structure of the collagenous matrix. Examples of these dynamic cell-matrix associations in tissue development have been revealed in the differentiation of certain types of cells, such as osteoblasts and cardiac myocytes. Collagen can also direct integrins to initiate a cascade which produces matrix metalloproteinases, thereby allowing degradation of the collagen matrix and permitting keratinocyte migration during wound-healing.

The structure of collagen imparts mechanical strength to tissues of the bone, tendon, and cartilage, while enabling the binding of other extracellular matrix proteins and soluble factors. It contains individual units that self-assemble into higher-order structures, and these properly-assembled three-dimensional structures are important in cellular recognition. Indeed, many biomaterials shown to successfully impart cellular function are fibers with structure and dimensions reminiscent of correctly-assembled collagen. Defects in collagen's structure or stability have been implicated in the development of various diseases, such as rheumatoid arthritis, varicose veins, osteogenesis imperfecta, and Ehlers-Danlos syndrome.

Since collagen is naturally the most abundant protein and is known to dynamically interact with cells, a collagen-based material potentially can mimic “natural” characteristics better than purely synthetic systems. The design and synthesis of such molecules, however, has exhibited challenges due to the stringent need to correctly post-translationally modify the polymeric backbone, the length of the collagen genes, and the repetitive nature of the polymeric units.

### SUMMARY

Provided herein is an isolated polynucleotide encoding a collagen-like polypeptide domain, wherein: at least 50% of the encoded collagen-like polypeptide domain is in the form of Gly-X-Y triads, where X and Y symbolize individual amino acids; X is proline in at least 20% of said triads; Y is proline in at least 20% of said triads; said polynucleotide encodes a region having at least 50 consecutive amino acids, wherein said region is at least 90% identical to an amino acid sequence of a naturally occurring collagen polypeptide; and no more than 98% of the nucleotides of the collagen-like polypeptide domain-encoding portion of said polynucleotide fall into a window of 100 consecutive nucleotides wherein the nucleotides of said window have greater than 98% identity to a naturally occurring collagen-encoding nucleotide sequence. Also provided herein is an isolated nucleic acid sequence encoding a collagen-like polypeptide domain, wherein: at least 50% of the encoded collagen-like polypeptide domain is in the form of Gly-X-Y triads, where X and Y symbolize individual amino acids; X is proline in at least 20% of said triads; Y is proline in at least 20% of said triads; said nucleotide sequence is configured to be specifically amplifiable and specifically mutatable; and no more than 98% of the nucleotides of the collagen-like polypeptide domain-encoding portion of said polynucleotide fall into a window of 100 consecutive nucleotides wherein the nucleotides of said window have greater than 98% identity to a naturally occurring collagen-encoding nucleotide sequence. In some such polynucleotides, the encoded polypeptide is capable of assembling into a triple-helical structure upon hydroxylation of said prolines in the Y position of said Gly-X-Y triads. Some such polynucleotides further comprise at least 2, at least 3, least 4, at least 5, least 6, at least 7, least 8, at least 9, least 10, or at least 12, endonuclease restriction sites not present at the corresponding site in a wild type mammalian collagen-encoding gene sequence. Some such polynucleotides further comprise at least 2, at least 3, least 4, at least 5, least 6, at least 7, least 8, at least 9, least 10, or at least 12, endonuclease restriction sites distributed throughout said gene at known distances from each other in the nucleotide sequence. In some such polynucleotides, said nucleotide sequence encodes at least 10, 20, 50, 100, or 200 consecutive amino acids of a naturally occurring collagen polypeptide core helical domain. In some such polynucleotides, the nucleotide sequence of the collagen-like polypeptide domain-encoding portion of said polynucleotide has less than 70% sequence identity with the nucleotide sequence of naturally-occurring collagen as set forth in SEQ ID NO: 1. In some such polynucleotides, said encoded collagen-like polypeptide domain is at least 100, 200, 300, 400, 500, 600, 700, 800, 900, 1000, 1100, 1200, 1300, 1400 or 1500 amino acids in length. Some such polynucleotides are configured for expression in yeast, bacteria, insect or mammalian cells. In some such polynucleotides, said polynucleotide further encodes a non-triple helical domain from a naturally occurring collagen. Also provided are yeast, bacteria, insect or mammalian host cells comprising the polynucleotide provided herein. In some such cells, said polynucleotide is integrated into the genome of the host organism.

Also provided are isolated polypeptides comprising an amino acid sequence at least 70% identical to a naturally occurring collagen polypeptide and comprising more than 3, 4, 5, 6, 7, 8, 9 or 10 amino acid changes. In some such polypeptides, said amino acid changes are each independently selected from the group consisting of: a protease recognition site, an integrin binding site, a kinase target site, growth factor binding site, cell binding site, and chemical attachment site. Also provided are isolated polypeptides comprising a collagen-like polypeptide domain, wherein: at least 50% of the encoded collagen-like polypeptide domain is in the form of Gly-X-Y triads, where X and Y symbolize individual amino acids; X is proline in at least 20% of said triads; Y is proline in at least 20% of said triads; and said collagen-like polypeptide domain is at least 300, 400, 500, 600, 700, 800, or 900, 1000, 1100, 1200, 1300, 1400 or 1500 amino acids in length.

Also provided are synthetic DNA molecules encoding a polypeptide that is at least 70% identical to a mature, full-length collagen polypeptide, wherein said DNA molecule has no more than 99%, 98%, 97%, 96%, or 95% sequence identity to the naturally occurring nucleotide sequence encoding said collagen polypeptide. Some such synthetic DNA molecules further encode a collagen C- or N-terminal propeptide. Some such synthetic DNA molecules further encode collagen C- and N-terminal propeptides. In some such synthetic DNA molecules, a signal sequence is attached. In some such synthetic DNA molecules, said mature, full-length collagen polypeptide is human collagen I, II, III, IV, V or XI.

Also provided are the synthetic DNA molecules of any of SEQ ID NOs: 3, 5, 7, 9 or 11.

Also provided are methods of making the synthetic DNA molecule or polynucleotide provided herein, comprising: synthesizing two or more polynucleotide fragments of said synthetic DNA molecule; and assembling said two or more polynucleotide fragments into the full-length synthetic DNA molecule. Also provided are methods of making a vector encoding collagen, comprising introducing the synthetic DNA or polynucleotide provided herein into a vector, wherein said introduced synthetic DNA is operatively linked to a regulatory sequence.

### BRIEF DESCRIPTION OF THE DRAWINGS

FIG. 1 depicts the nucleotide sequence (SEQ ID NO: 3) encoding the amino acid sequence of the human collagen II alpha middle helical domain (SEQ ID NO:4), and the oligonucleotide synthesis and assembly strategy for SEQ ID NO: 3.

FIG. 2 depicts the nucleotide sequence (SEQ ID NO: 5) encoding the amino acid sequence of the human collagen 1 alpha middle helical domain (SEQ ID NO:6), and the oligonucleotide synthesis and assembly strategy for SEQ ID NO: 5.

FIG. 3 depicts the nucleotide sequence (SEQ ID NO: 7) encoding the amino acid sequence of the human collagen III alpha middle helical domain, with two amino acid substitutions to add cysteine residues at a285c and g581c (SEQ ID NO:6), and the oligonucleotide synthesis and assembly strategy for SEQ ID NO: 7.

FIG. 4 depicts the nucleotide sequence (SEQ ID NO: 9) encoding the amino acid sequence of the human collagen III alpha middle helical domain, with putative integrin binding sites (GRPGER, GAPGER, GAPGER, GMPGER) substituted with the sequence GSPGGK, and with the sequence GLKGENGLPGEN substituted with the sequence GSPGGKGSPGGK (see SEQ ID NO:10), and the oligonucleotide synthesis and assembly strategy for SEQ ID NO: 9.

FIG. 5 depicts the nucleotide sequence (SEQ ID NO: 11) encoding the amino acid sequence of the human collagen III alpha middle helical domain of SEQ ID NO:9, with a heterologous collagen I integrin binding site-containing sequence GERGFPGERGVQ inserted for the sequence GRPGRPGERGLP (see SEQ ID NO:12), and the oligonucleotide synthesis and assembly strategy for SEQ ID NO: 11.

FIG. 6 depicts schematic designs of collagen III wild type, integrin binding site-inserted, and proteolytic site-inserted sequences, where the design can be modular and contain the integrin binding sites and proteolytic sites at regular intervals, or in various combinations.

FIG. 7 depicts an electrophoretic pattern showing assembly of the full-length collagen III encoding polynucleotide (lane 7) and the collagen middle helical domain encoding polynucleotide (lane 8).

FIG. 8 depicts the nucleotide sequence encoding the amino acid sequence of the N-terminal portion of human collagen III alpha, including up to the first three amino acids of the middle helical domain (SEQ ID NO: 14), and the oligonucleotide synthesis and assembly strategy for SEQ ID NO: 13.

FIG. 9 depicts the nucleotide sequence (SEQ ID NO: 15) encoding the amino acid sequence of the middle helical domain of human collagen III alpha, including the last twenty amino acids of the N-terminal portion and the first twenty amino acids of the C-terminal portion (SEQ ID NO:16), and the oligonucleotide synthesis and assembly strategy for SEQ ID NO: 15.

FIG. 10 depicts the nucleotide sequence (SEQ ID NO: 17) encoding the amino acid sequence of the C-terminal portion of human collagen III alpha, including up to the last three amino acids of the middle helical domain (SEQ ID NO:18), and the oligonucleotide synthesis and assembly strategy for SEQ ID NO: 17.

FIG. 11 depicts the nucleotide sequence (SEQ ID NO: 1) and amino acid sequence (SEQ ID NO:2) for wild type human collagen III.

### DETAILED DESCRIPTION

The ability to create artificial scaffolds that can direct the activity of cells is a critical component of furthering the areas of biomimetic materials, especially those associated with regenerative medicine and therapies for disease. Since collagen is naturally the most abundant protein and is known to dynamically interact with cells, a collagen-based material potentially can mimic “natural” characteristics better than purely synthetic systems. The design and synthesis of such molecules, however, has exhibited challenges not associated with other protein-based polymers, primarily because of the stringent need to correctly post-translationally modify the polymeric backbone, the length of the collagen genes, and the repetitive nature of the polymeric units. Provided herein are sequences, polynucleotides, polypeptides and related compositions and methods of collagen or collagen-like molecules which can be readily manipulated by known molecular methodologies to thereby provide collagen proteins as a platform for various applications.

Provided herein are nucleotide sequences and isolated polynucleotides encoding a collagen-like polypeptide domain. The collagen-like polypeptide domains encoded thereby are polypeptide domains that demonstrate a structural characteristic of a naturally occurring collagen. For example, naturally occurring collagen is known to be glycine-rich and contain a large number of Gly-X-Y repeats. In another example, naturally occurring collagen is known to form left-handed triple helices, for example, upon hydroxylation of proline residues therein. In another example, naturally occurring collagen is known to form gelatin, for example upon partial hydrolysis. Thus, for example, a collagen-like polypeptide domain is a domain that can be glycine-rich with Gly-X-Y repeats, or can form a left-handed triple-helix, or gelatin, under conditions similar to conditions in which naturally occurring collagen forms a left-handed triple-helix, or gelatin. As one example, a collagen-like polypeptide can demonstrate a structural characteristic of collagen Iα by being capable of forming a triple-helix in the presence of collagen Iβ. As another example, a collagen-like polypeptide can demonstrate a structural characteristic of collagen IIα by being capable of forming a triple-helix in the presence of collagen IIβ. As another example, a collagen-like polypeptide can demonstrate a structural characteristic of collagen III by being capable of forming a triple-helix.

In some embodiments, the encoded collagen-like polypeptide domain contains Gly-X-Y triads, where “Gly” is the amino acid glycine, and X and Y symbolize individual amino acids. When the encoded collagen-like polypeptide domain contains Gly-X-Y triads, at least, or at least about, 20%, 25%, 30%, 35%, 40%, 45%, 50%, 55%, 60%, 65%, 70%, 75%, 80%, 85%, 90%, 95%, 96%, 97%, 98%, or 99% of the encoded collagen-like polypeptide domain can be in the form of Gly-X-Y triads. It will be understood by those skilled in the art that the above can refer to only a subset of the entire sequence of the encoded polypeptide, where additional portions of the encoded polypeptide can include Gly-X-Y triad regions, non-Gly-X-Y triad collagen-like regions, and non-collagen-like regions.

When the encoded collagen-like polypeptide domain contains Gly-X-Y triads, X can be proline in at least, or at least about, 5%, 8%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of the triads. In addition, when the encoded collagen-like polypeptide domain contains Gly-X-Y triads, Y can be proline in at least, or at least about, 5%, 8%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of the triads.

In some embodiments, the polynucleotide encodes a polypeptide region that is sequence similar to an amino acid sequence of a naturally occurring collagen polypeptide. In some such embodiments, the encoded polypeptide region is at least, or at least about, 50%, 55%, 60%, 65%, 70%, 75%, 80%, 85%, 90%, 95%, 96%, 97%, 98%, 99%, 99.5%, 99.8%, or 99.9% identical after alignment to an amino acid sequence of a naturally occurring collagen polypeptide. In some such embodiments, the encoded polypeptide region is 100% identical after alignment to an amino acid sequence of a naturally occurring collagen polypeptide. In some such embodiments, the encoded polypeptide region that is sequence similar to an amino acid sequence of a naturally occurring collagen polypeptide is at least, or at least about, 5, 8, 10, 15, 20, 25, 30, 35, 40, 45, 50, 55, 60, 65, 70, 75, 80, 85, 90, 95, 100, 110, 120, 130, 140, 150, 175, 200, 225, 250, 275, 300, 350, 400, 450, 500, 600, 700, 800, 900, or 1000 consecutive amino acids in length. In some embodiments, the encoded amino acid sequence is identical after alignment over the entire length of a naturally occurring collagen polypeptide. It will be understood by those skilled in the art that the identity calculation is based on known methods such as BLAST.

In other embodiments, the polynucleotide encodes a polypeptide region that is not sequence similar to an amino acid sequence of a naturally occurring collagen polypeptide. In some such embodiments, the encoded polypeptide does not contain a region more than, or more than about, 5%, 10%, 15%, 20%, 25%, 30%, 35%, 40%, 45%, 50%, 55%, 60%, 65%, 70%, 75%, 80%, 85%, or 90% identical after alignment to an amino acid sequence of a naturally occurring collagen polypeptide. In some such embodiments, the encoded polypeptide region having the above maximum level of sequence similarity to an amino acid sequence of a naturally occurring collagen polypeptide is at least, or at least about, 5, 8, 10, 15, 20, 25, 30, 35, 40, 45, 50, 55, 60, 65, 70, 75, 80, 85, 90, 95, 100, 110, 120, 130, 140, 150, 175, 200, 225, 250, 275, 300, 350, 400, 450, 500, 600, 700, 800, 900, or 1000 consecutive amino acids in length. For example, in some embodiments, no encoded region at least 50 amino acids in length is more than 50% identical after alignment to a naturally occurring collagen polypeptide.

As provided herein, the nucleotide sequence of the polynucleotide encoding the collagen-like polypeptide domain is not identical to any naturally occurring nucleotide sequence, such as a naturally occurring collagen polypeptide-encoding nucleotide sequence. The degree to which the polynucleotide sequences provided herein vary from a naturally occurring sequence can vary according to the desired modifications to be made in accordance with the teachings provided herein. The degree to which the polynucleotide sequences provided herein vary from a naturally occurring sequence can be determined in any of a variety of ways. One such manner for evaluating the difference between the polynucleotide sequences provided herein and a naturally occurring sequence is to compare the identity after alignment of a provided sequence to a naturally occurring sequence over a window of consecutive nucleotides of a specified length. A maximum level of sequence identity can be established to a naturally occurring polynucleotide sequence as calculated within any such window that can be placed over the polynucleotide sequence. Thus, a maximum level of sequence identity after alignment to a naturally occurring sequence is established for any particular window of the nucleotide sequence. The window size can vary as desired by the skilled artisan, and will typically be, or will typically be about, 10, 20, 30, 40, 50, 60, 70, 80, 90, 100, 110, 120, 130, 140, 150, 160, 170, 180, 200, 220, 240, 260, 280, 300, 350, 400, 450, 500, 550, 600, 700, 800, 900, 1000, 1100, 1200, 1300, 1400, 1500, 1600, 1700, 1800, 1900, 2000, 2200, 2400, 2600, 2800, or 3000 nucleotides in length. The maximum level of sequence identity within the window can be set by those skilled in the art in accordance with the teachings provided herein. Typically, this level will be no more than, or no more than about, 50%, 55%, 60%, 65%, 70%, 75%, 80%, 85%, 90%, 95%, 96%, 97%, 98%, 99%, 99.5%, 99.8%, or 99.9% of the nucleotides. The maximum level of sequence identity after alignment to a naturally occurring sequence also can be set by those skilled in the art in accordance with the teachings provided herein. Typically, no more than the above-listed percentage of nucleotides fall into an above-specified length window in which the nucleotides have greater than, or greater than about, 50%, 55%, 60%, 65%, 70%, 75%, 80%, 85%, 90%, 95%, 96%, 97%, 98%, 99%, 99.5%, 99.8%, or 99.9% sequence identity after alignment to a naturally occurring sequence such as a naturally occurring collagen-encoding nucleotide sequence. In one example, no more than 98% of the nucleotides of the collagen-like polypeptide domain-encoding portion of said polynucleotide fall into a window of 100 consecutive nucleotides wherein the nucleotides of said window have greater than 98% sequence identity to a naturally occurring collagen-encoding nucleotide sequence.

In some embodiments, the nucleotide sequence is configured to be specifically amplifiable and specifically mutatable. A specifically amplifiable and specifically mutatable nucleotide sequence is a nucleotide sequence that is sufficiently non-repetitive so as to permit an oligonucleotide that is 100% complementary to a selected portion of the nucleotide sequence to hybridize to the selected portion with greater affinity than to any other portion of the nucleotide sequence. In another embodiment, such an oligonucleotide is at least, or at least about, 5, 6, 7, 8, 9, 10, 11, 12, 13, 14, 15, 16, 17, 18, 19, 20, 22, 24, 26, 28, 30, 35, 40, 45, 50, 65, 70, 75, 80, 85, 90, 95, 100, 110, 120, 130, 140, 150, 160, 170, 180, or 200 bases in length. In one embodiment, such an oligonucleotide is a chemically synthesized oligonucleotide. The amount of greater affinity can be established according to the requirements of the molecular technique implemented by one skilled in the art. In one embodiment, the greater affinity to the selected portion compared to any other portion of the nucleotide sequence is reflected by a differential of at least, or at least about, 0.5° C., 1° C., 1.5° C., 2° C., 2.5° C., 3° C., 3.5° C., 4° C., 4.5° C., 5° C., 5.5° C., 6° C., 6.5° C., 7° C., 7.5° C., 8° C., 8.5° C., 9° C., 9.5° C., or 10° C. between the melting temperature of the oligonucleotide hybridized to its intended sequence (the selected portion of the nucleotide sequence), compared to the highest melting temperature of an unintended hydridization to the rest of the polynucleotide (any other portion of the nucleotide sequence). Heretofore, methods of generating synthetic collagen-encoding nucleotide sequences suffered from the limitation of having large number of repeats throughout, which eliminates the possibility of amplifying the encoding polynucleotide with fidelity and prevents site specific mutagenesis, resulting in a synthetic collagen-encoding nucleotide sequences lacking the flexibility of being amenable to manipulation by traditional molecular biological methodologies. As provided herein, this problem has been solved by developing a nucleotide sequence, and a manner for generating such nucleotide sequence, that permits any portion of the synthetic collagen-encoding nucleotide sequence to be uniquely targeted by an appropriate primer or other oligonucleotide.

In some embodiments, the nucleotide sequence is configured to be assembled by a plurality of oligonucleotides smaller than the entire size of the final polynucleotide, while avoiding deleterious incorrect hybridization by the oligonucleotides. Olignucleotide-based assembly of polynucleotides is known in the art, as described elsewhere herein. When oligonucleotide design is performed in accordance with the methods provided herein, the oligonucleotides assemble as designed while avoiding deleterious incorrect hybridization because the oligonucleotides are designed to hybridize to their intended hybridization partner oligonucleotide with a differential of at least, or at least about, 0.5° C., 1° C., 1.5° C., 2° C., 2.5° C., 3° C., 3.5° C., 4° C., 4.5° C., 5° C., 5.5° C., 6° C., 6.5° C., 7° C., 7.5° C., 8° C., 8.5° C., 9° C., 9.5° C., or 10° C. between the melting temperature of the oligonucleotide hybridized to its intended hybridization partner oligonucleotide, compared to the highest melting temperature of a hydridization to an unintended hybridization partner oligonucleotide. The lengths of such oligonucleotides can vary, but are typically at least, or at least about, 10, 11, 12, 13, 14, 15, 16, 17, 18, 19, 20, 22, 24, 26, 28, 30, 35, 40, 45, 50, 65, 70, 75, 80, 85, 90, 95, 100, 110, 120, 130, 140, 150, 160, 170, 180, 200, 220, 240, 260, 280, or 300 bases in length.

In some embodiments, the collagen-like polypeptide encoding polynucleotide is at least, or at least about, 600, 700, 800, 900, 1000, 1100, 1200, 1300, 1400, 1500, 1600, 1700, 1800, 1900, 2000, 2200, 2400, 2600, 2800, 3000, 3200, 3400, 3600, 3800, 4000, 4200, 4400, 4600, 4800, or 5000 nucleotides in length. In one such embodiment, collagen-like polypeptide encoding polynucleotides with the above length can encode a polypeptide that is not, or does not contain a region that is, more than, or more than about, 5%, 10%, 15%, 20%, 25%, 30%, 35%, 40%, 45%, 50%, 55%, 60%, 65%, 70%, 75%, 80%, 85%, or 90% identical to an amino acid sequence of a naturally occurring collagen polypeptide.

In some embodiments, the encoded polypeptide is capable of assembling into a triple-helical structure upon hydroxylation of prolines in the Y position of said Gly-X-Y triads, under suitable conditions. As is known in the art, hydroxylation of prolines in the Y position of said Gly-X-Y triads in collagen polypeptides can result in triple helical formation of the collagen polypeptides. Such triple helices can be homo-trimers or hetero-trimers, as is known in the art. Conditions and methods for hydroxylation of prolines in vitro and in vivo are known in the art, and any such condition and method can be applied to the collagen-like polypeptides provided herein. Such conditions can include exposure to one or more naturally occurring collagen polypeptides with which collagen-like polypeptides provided herein can form a hetero-trimer upon proline hydroxylation. The collagen-like polypeptides provided herein can contain a core helical domain similar to or identical to a naturally occurring polypeptide, and, in this embodiment, the core helical domain can assemble as a homo-trimer and/or a hetero-trimer upon proline hydroxylation.

In some embodiments, polynucleotides provided herein contain at least, or at least about, 2, 3, 4, 5, 6, 7, 8, 9, 10, 12, 14, 16, 18, 20, 22, 25, 30, 35, 40, or 45 heterologous sequences not present at the corresponding site in a wild type collagen-encoding gene sequence. Such a corresponding site is a site of a wild type collagen-encoding gene sequence that aligns with the site of the polynucleotide provided herein after performing an alignment between the polynucleotide sequences. Alignments of sequences can be performed as known in the art, using, for example, BLAST or other suitable software. Such heterologous sequences can be any known heterologous sequences, as desired by one skilled in the art, and can include protease recognition sites, integrin binding sites, kinase target sites, growth factor binding sites, cell binding sites, chemical attachment sites, and endonuclease restriction sites. Such heterologous sequences can be the same or different (e.g., have different sequences), and will typically include at least, or at least about, 2, 3, 4, 5, 6, 7, 8, 9, 10, 12, 14, 16, 18, or 20 different heterologous sequences. In some embodiments, polynucleotides provided herein contain at least, or at least about, 2, 3, 4, 5, 6, 7, 8, 9, 10, 12, 14, 16, 18, 20, 22, 25, 30, 35, 40, or 45 heterologous sequences not present in a wild type mammalian collagen-encoding gene sequence.

In some embodiments, polynucleotides provided herein contain at least, or at least about, 2, 3, 4, 5, 6, 7, 8, 9, 10, 12, 14, 16, 18, 20, 22, 25, 30, 35, 40, or 45 heterologous sequences distributed throughout the polynucleotide at intentional distances from each other in the nucleotide sequence. Intentional distances are characterized in that the nucleotide sequence can be designed such that the identity of every nucleotide position is known, and therefore, the presence of the heterologous sequences is therefore intentional because of the design. For example, the position of the heterologous sequences can be designated upon design of the nucleotide sequence. For example, the positions of the heterologous sequences can be user-defined. In some embodiments, the distance between a plurality of heterologous sequences is set to be approximately the same. For example, the distance between heterologous sequences can be the same within 3%, 5%, 7%, 10%, 15%, or 20% variation from the mean distance between heterologous sequences. In some such embodiments, all heterologous sequences separated by approximately the same distance are the same (e.g., have the same nucleotide sequence). In some such embodiments, heterologous sequences separated by approximately the same distance are different (e.g., have different nucleotide sequences).

In some embodiments, the nucleotide sequence encodes a collagen-like polypeptide domain that can be at least, or at least about, 10, 20, 30, 40, 50, 60, 70, 80, 90, 100, 120, 140, 160, 180, 200, 250, 300, 350, 400, 450, 500, 550, 600, 650, 700, 750, 800, 850, 900, 950, 1100, 1200, 1300, 1400 or 1500 amino acids in length. Such a collagen-like polypeptide domain can be, for example, a collagen-like core helical domain. Such a region can be, for example, at least, or at least about, 10, 20, 30, 40, 50, 60, 70, 80, 90, 100, 120, 140, 160, 180, 200, 250, 300, 350, 400, 450, 500, 550, 600, 650, 700, 750, 800, 850, 900, 950, or 1000 consecutive amino acids of a naturally occurring collagen polypeptide core helical domain. In some such embodiments, the nucleotide sequence encodes a region identical to a naturally occurring collagen polypeptide core helical domain.

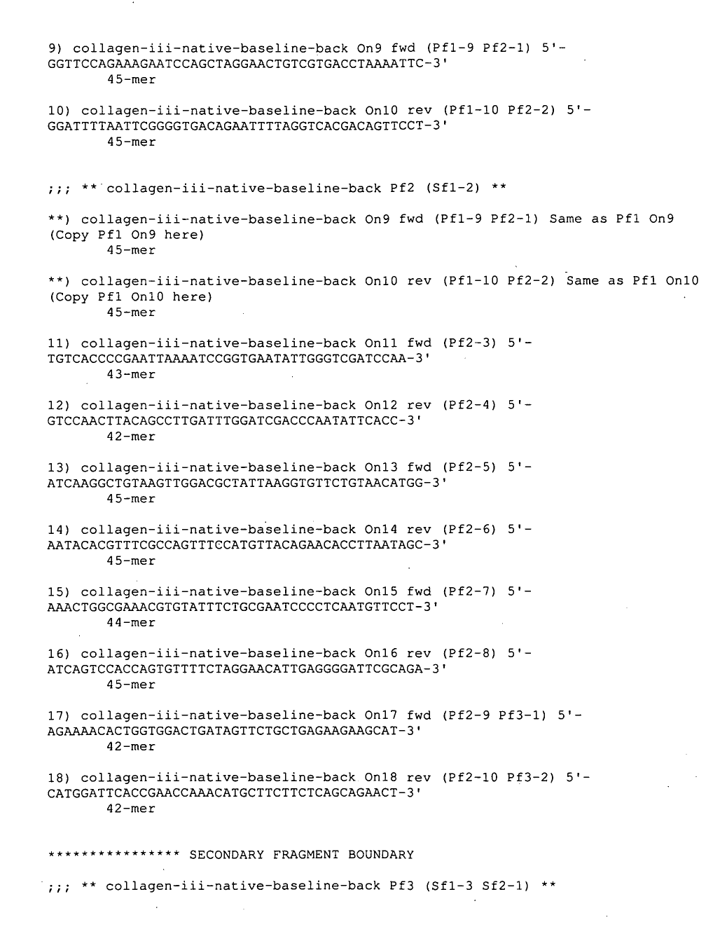

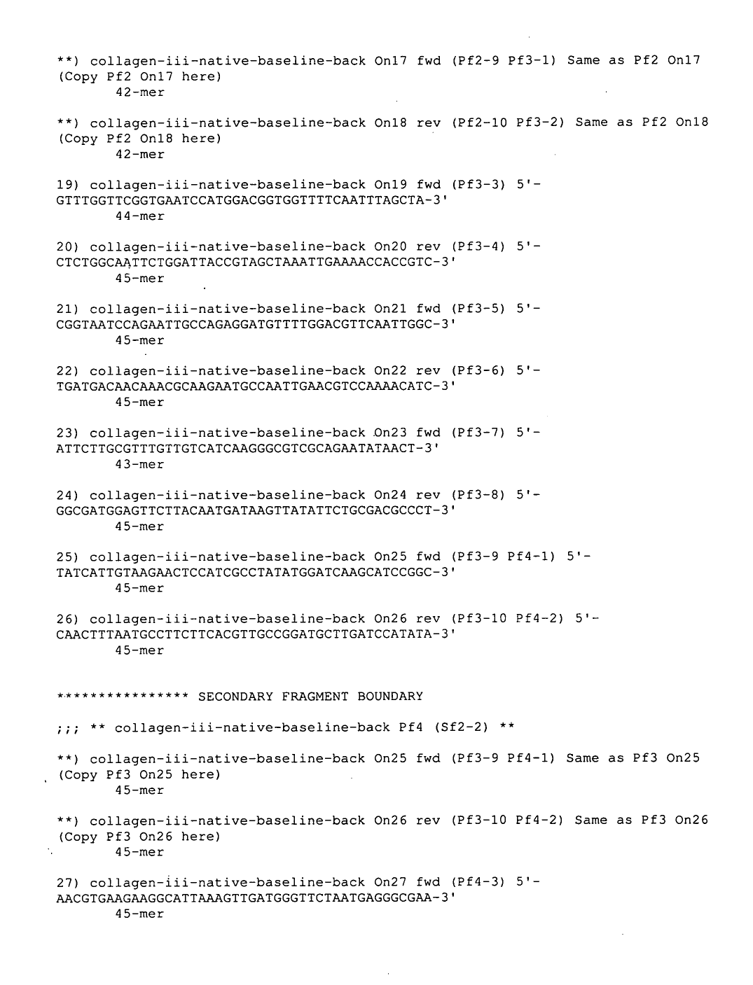

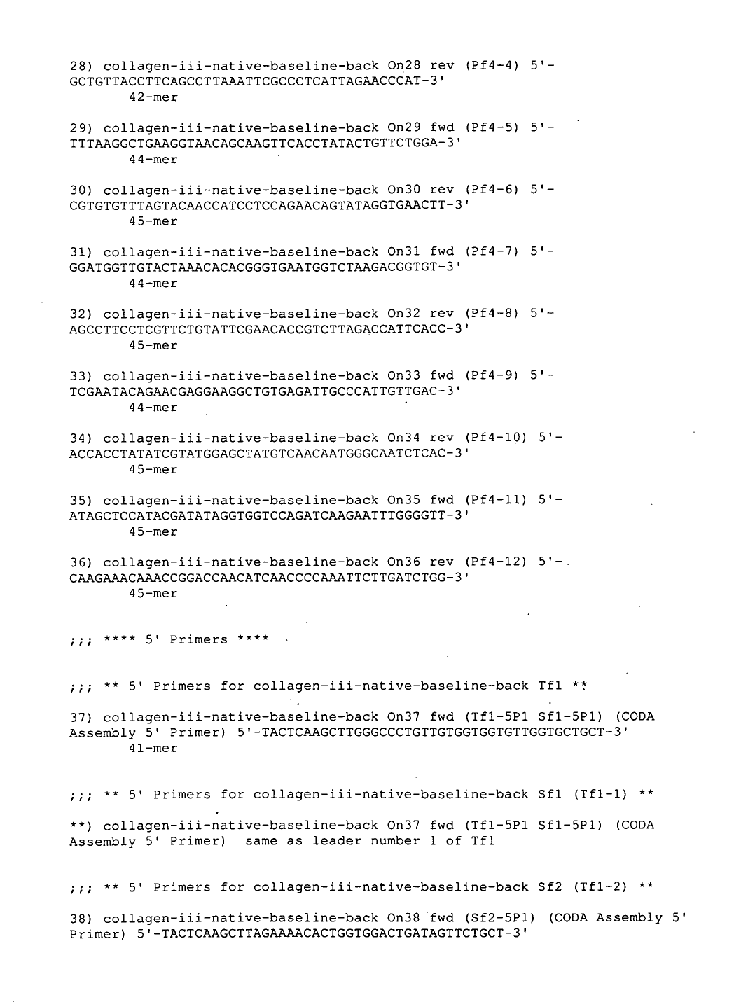

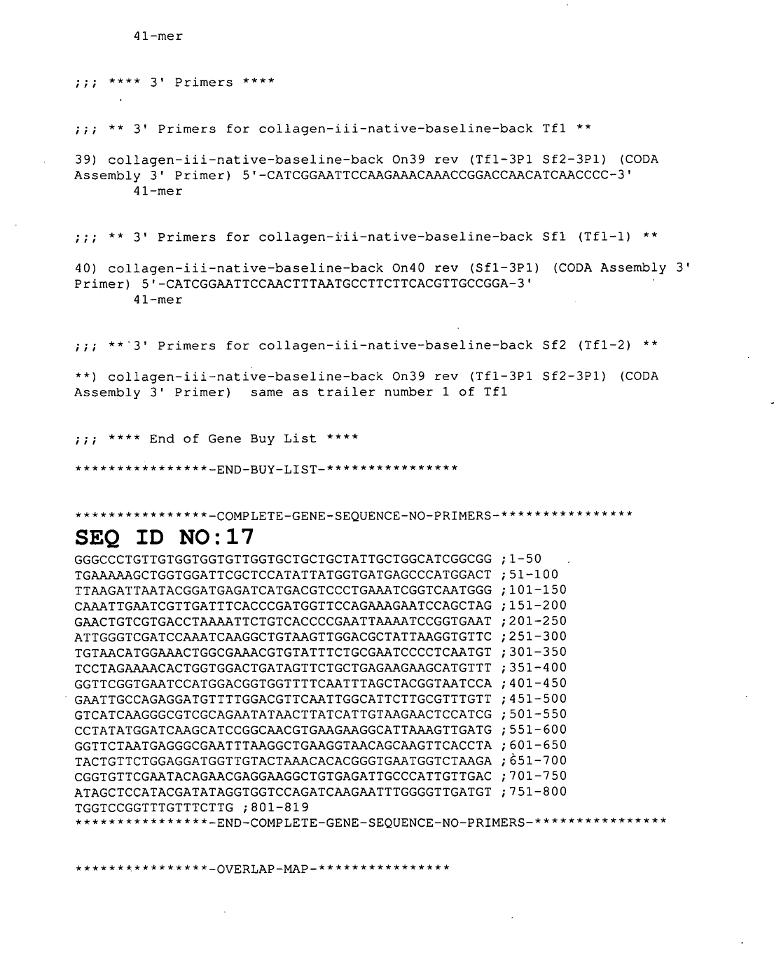

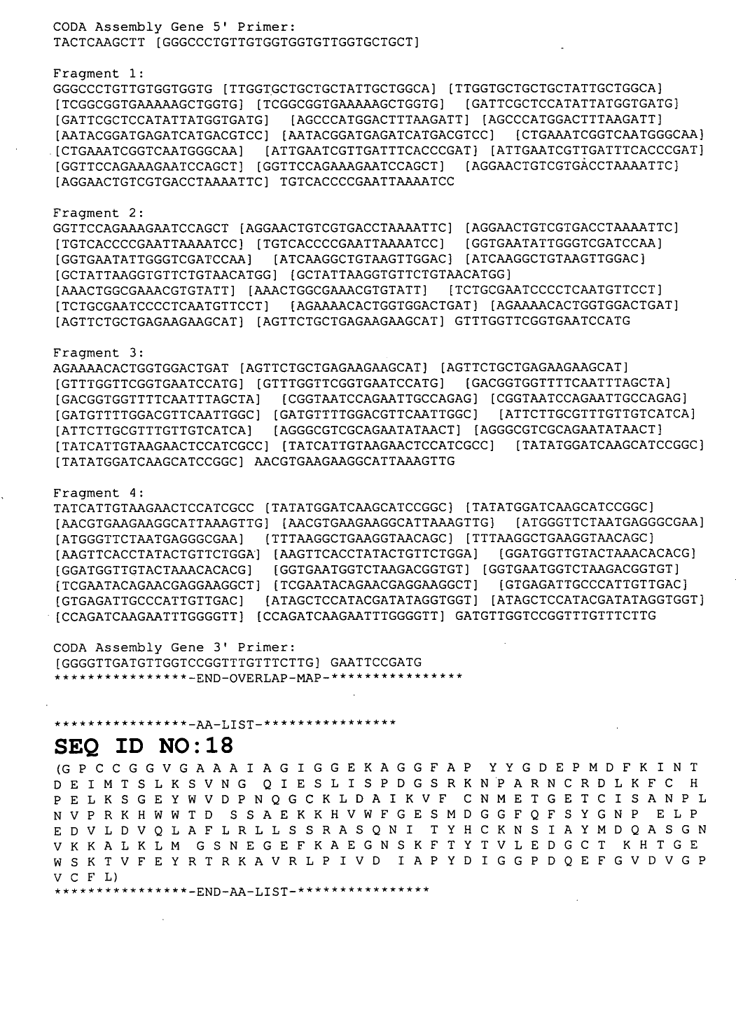

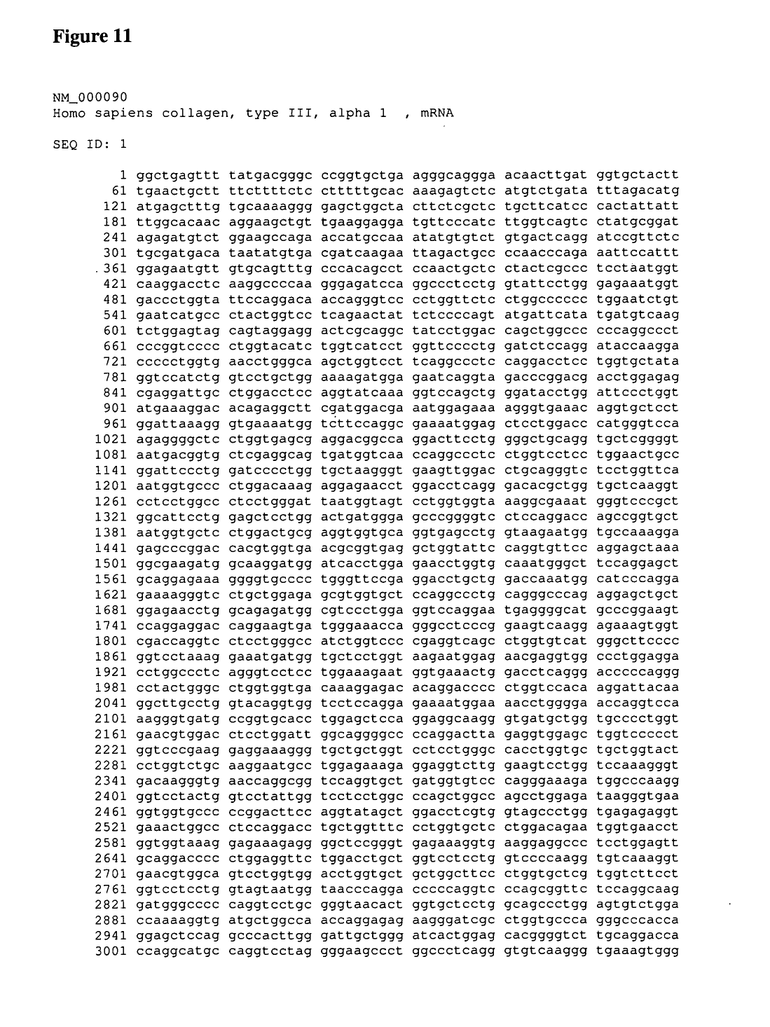

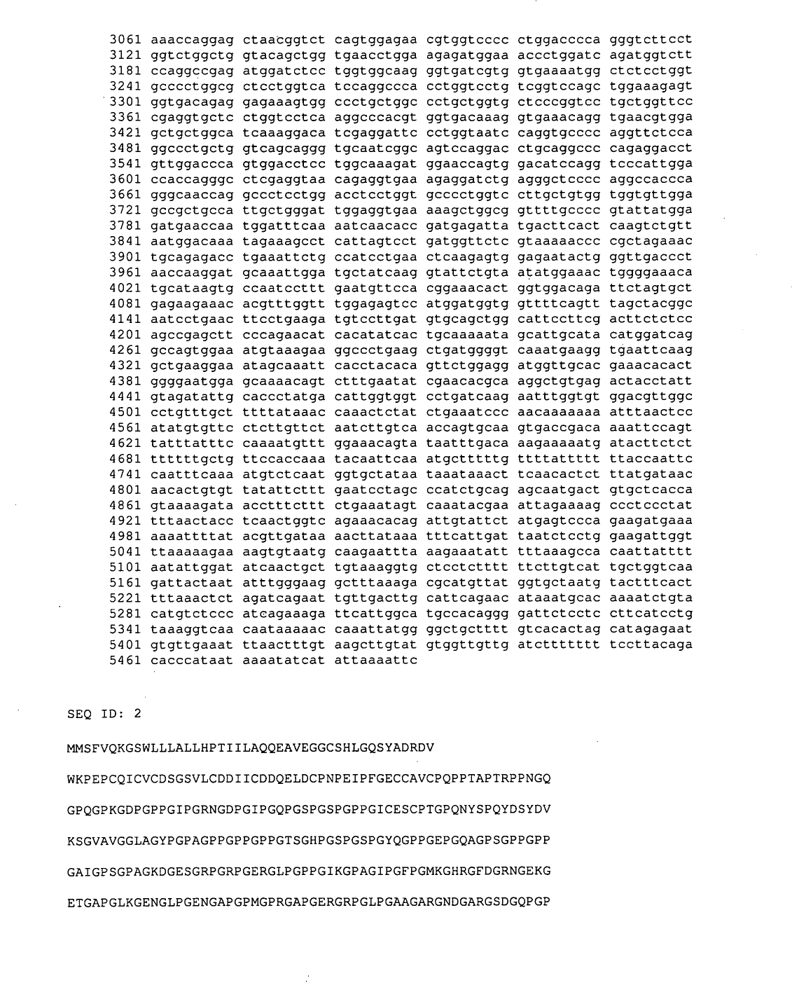

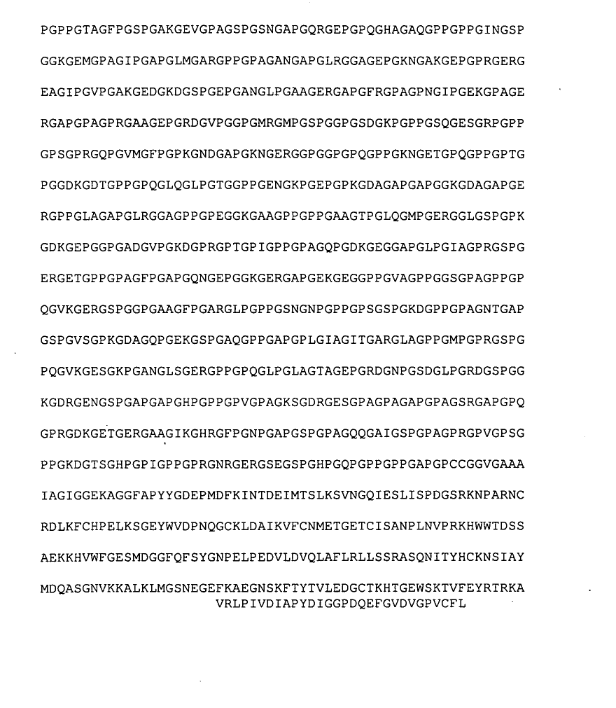

In some embodiments, the nucleotide sequence of the collagen-like polypeptide domain-encoding portion of the polynucleotide has little or no sequence similarity to a naturally-occurring collagen-encoding polynucleotide sequence. For example, the nucleotide sequence of the collagen-like polypeptide domain-encoding portion of the polynucleotide can have less than, or less than about, 99%, 98%, 97%, 96%, 95%, 90%, 85%, 80%, 75%, 70%, 65%, 60%, 55%, 50%, 45%, 40%, 35%, 30%, 25%, 20%, 15%, or 10%, sequence identity after alignment with a nucleotide sequence of naturally-occurring collagen. Exemplary known naturally-occurring collagens are provided elsewhere herein, and include human collagens I, II, III, IV, V, and XI, such as the sequence of human collagen III as set forth in SEQ ID NO: 1, and the encoded amino acid sequence of human collagen III is set for in SEQ ID NO:2 (see FIG. 11).

Also provided are synthetic DNA molecules encoding a polypeptide that is at least, or at least about, 50%, 55%, 60%, 65%, 70%, 75%, 80%, 85%, 90%, 91%, 92%, 93%, 94%, 95%, 96%, 97%, 98%, 99%, 99.5%, 99.8%, or 99.9% identical to the mature, full-length collagen polypeptide, wherein said DNA molecule has no more than, or no more than about, 99%, 98%, 97%, 96%, 95%, 90%, 85%, 80%, 75%, 70%, 65%, 60%, 55%, or 50% sequence identity to a naturally occurring nucleotide sequence encoding the collagen polypeptide.

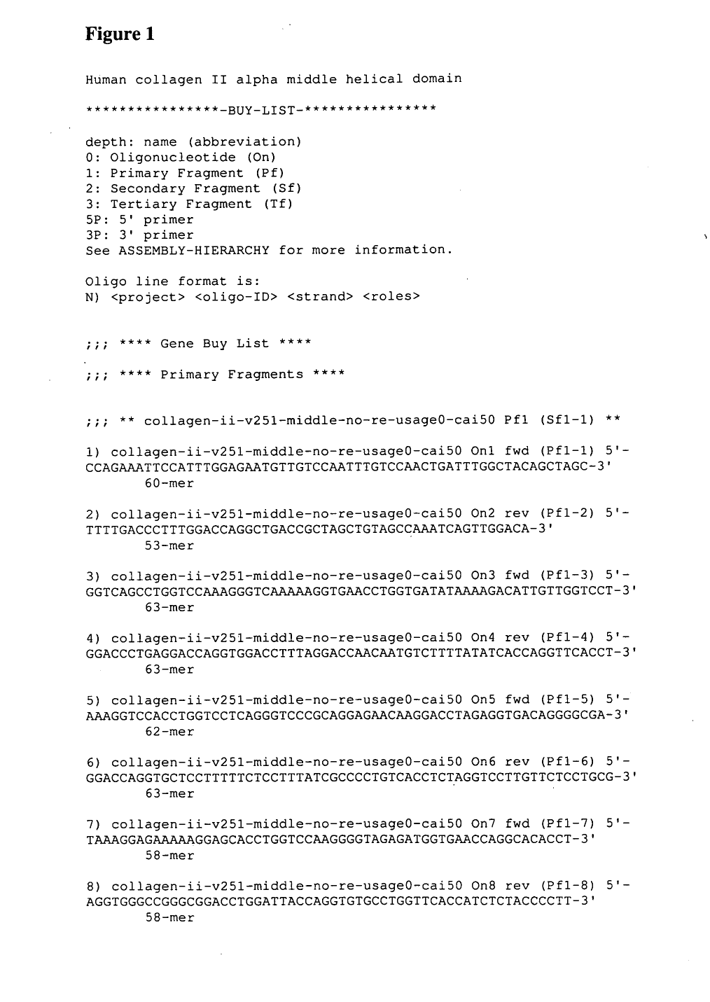

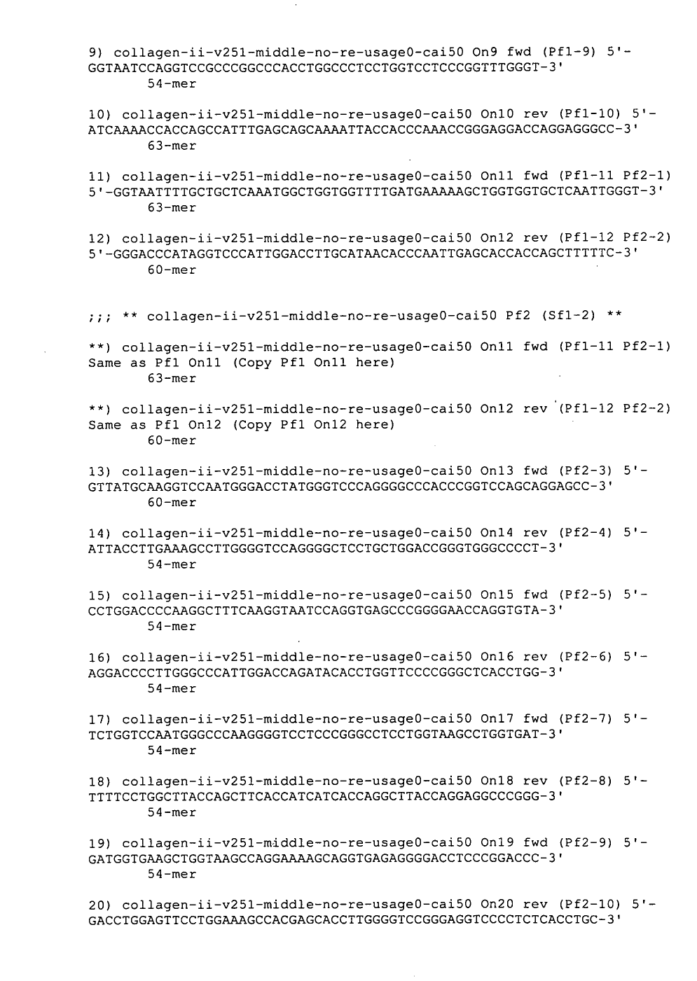

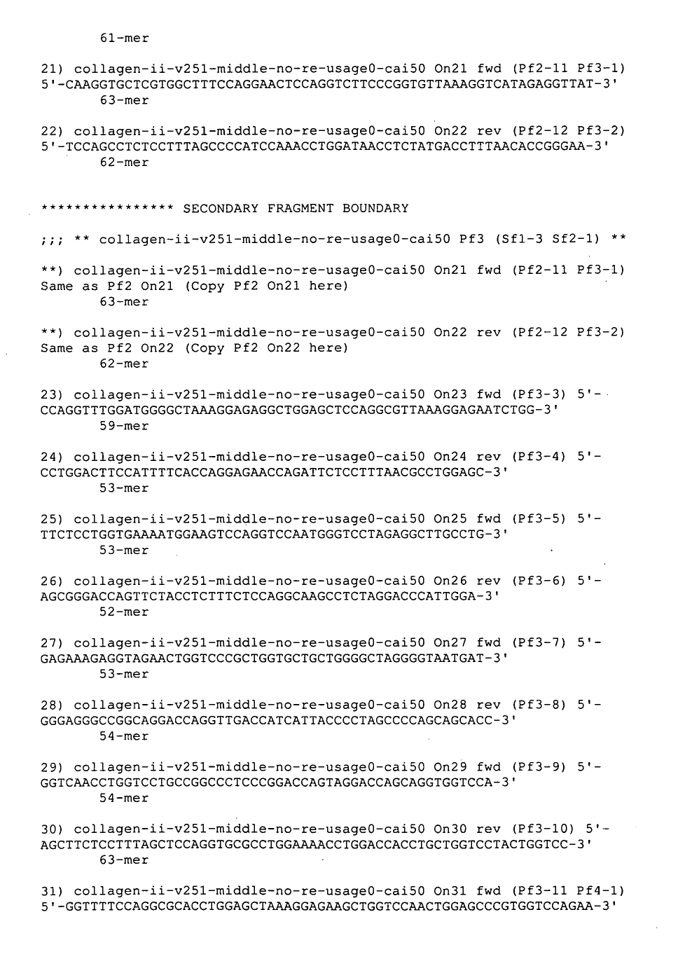

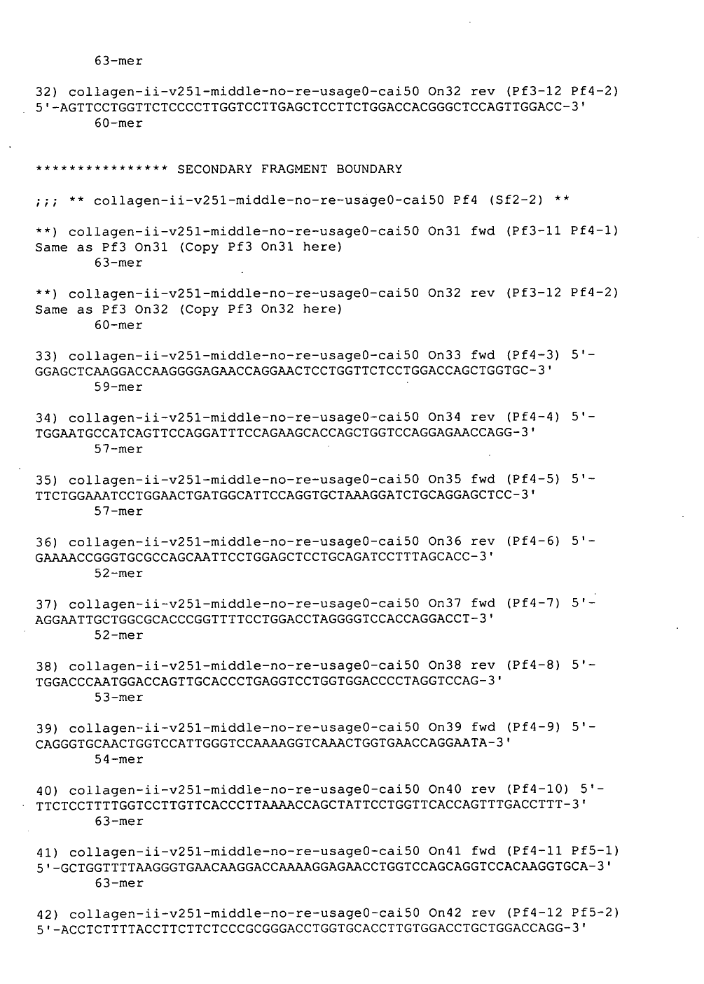

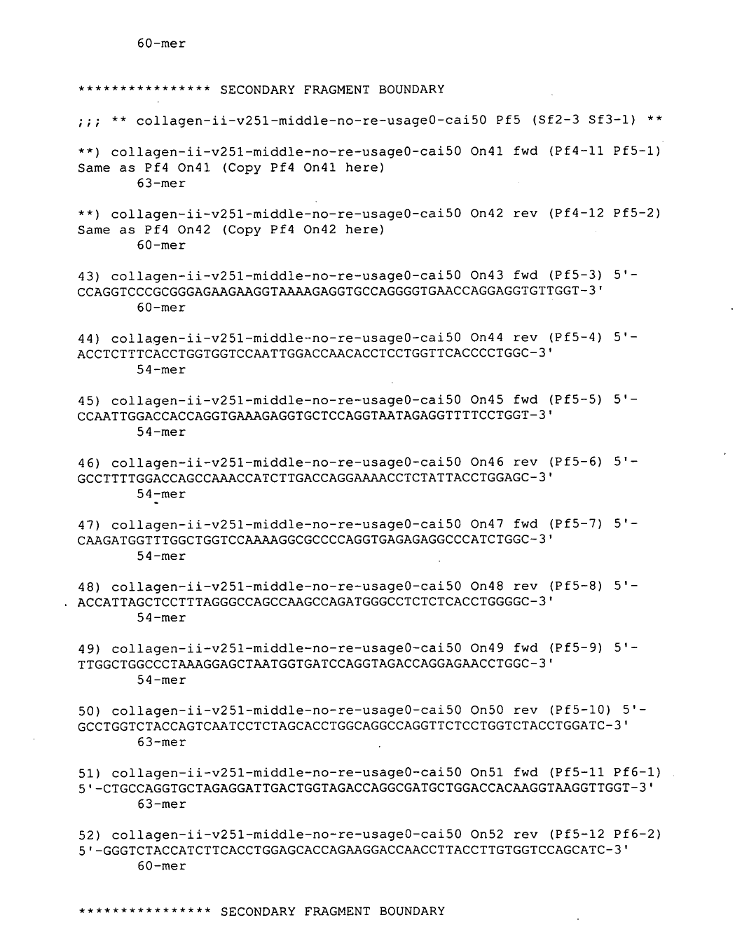

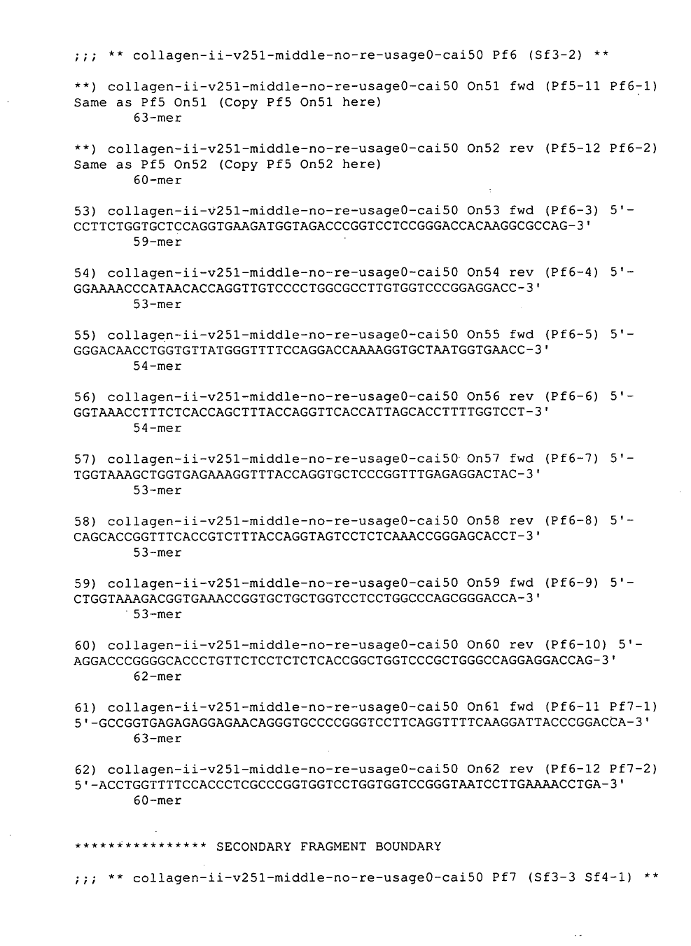

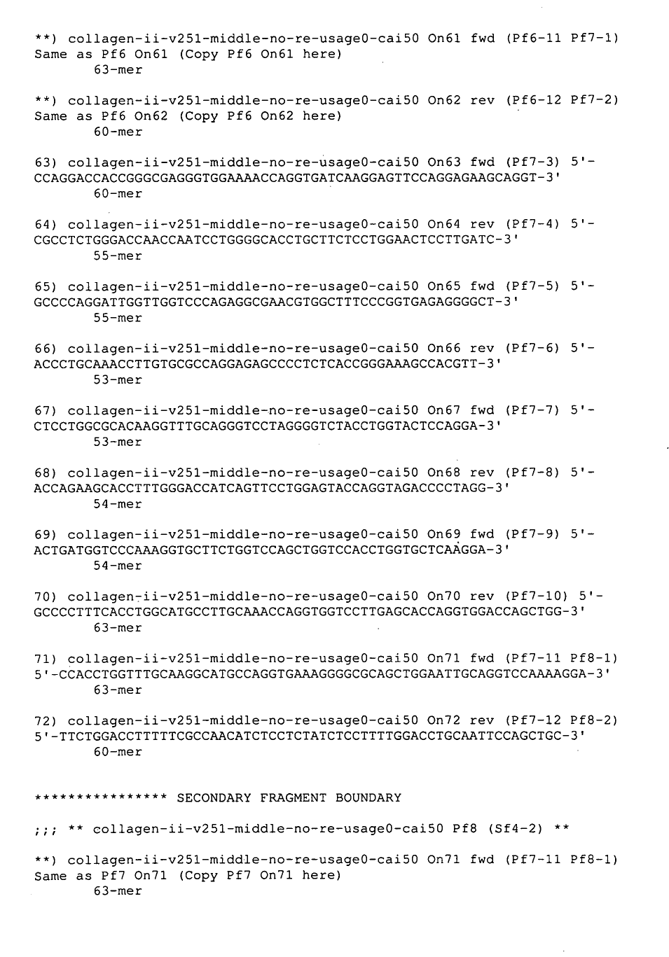

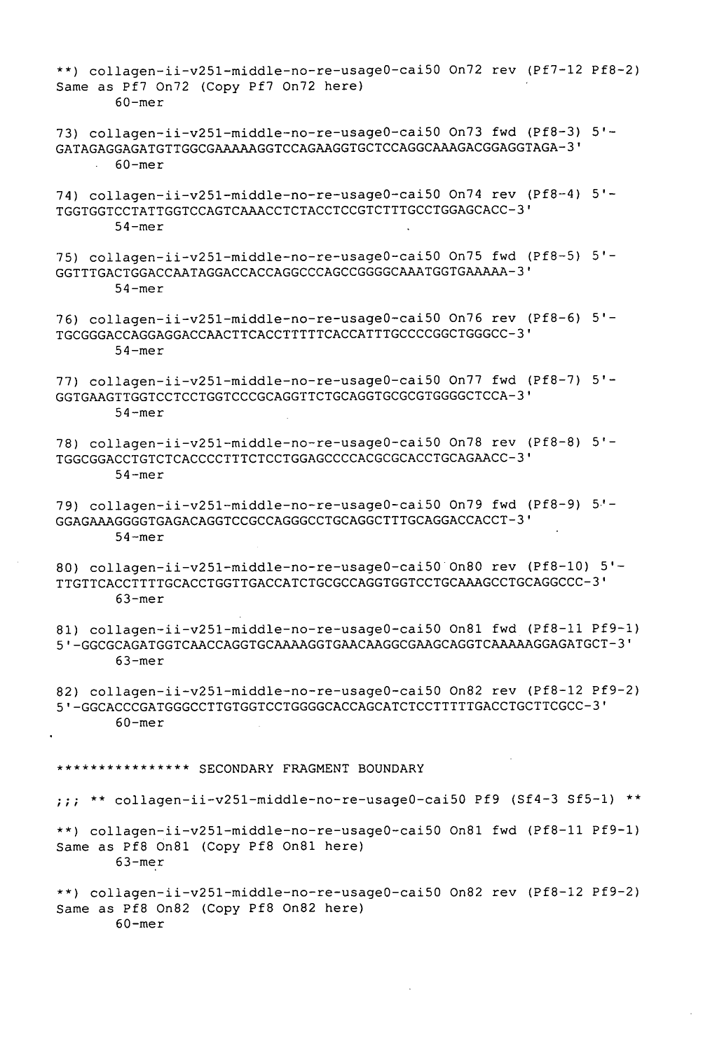

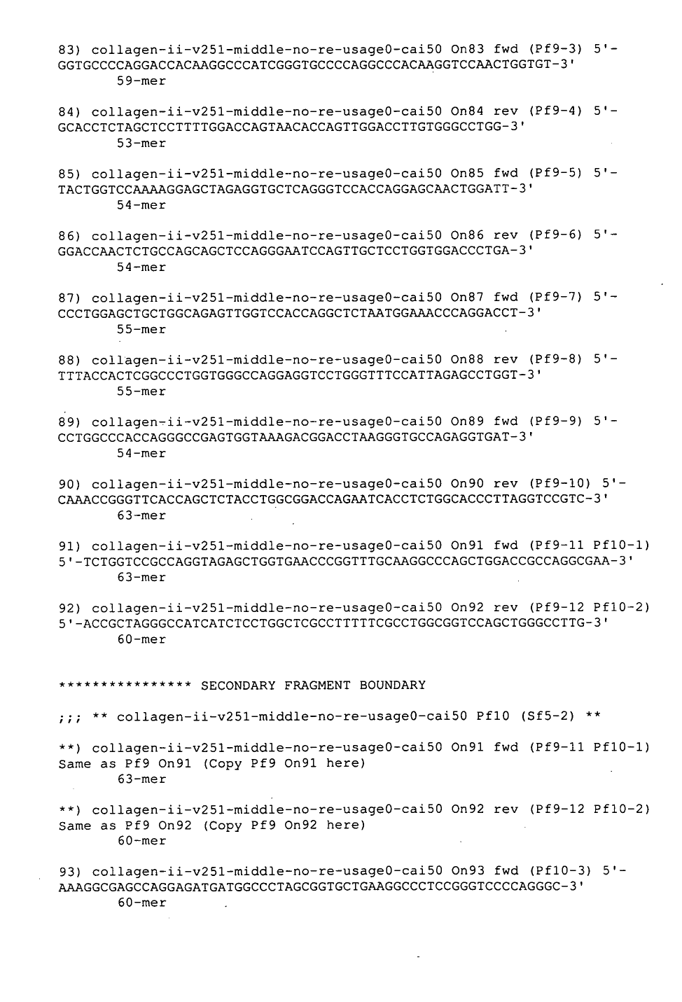

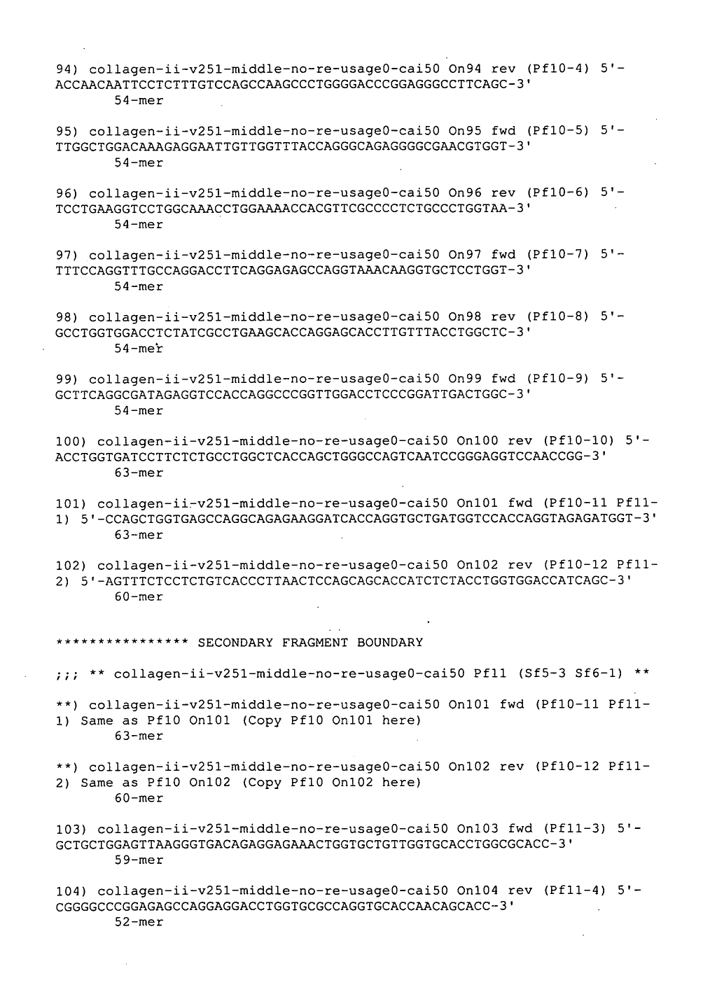

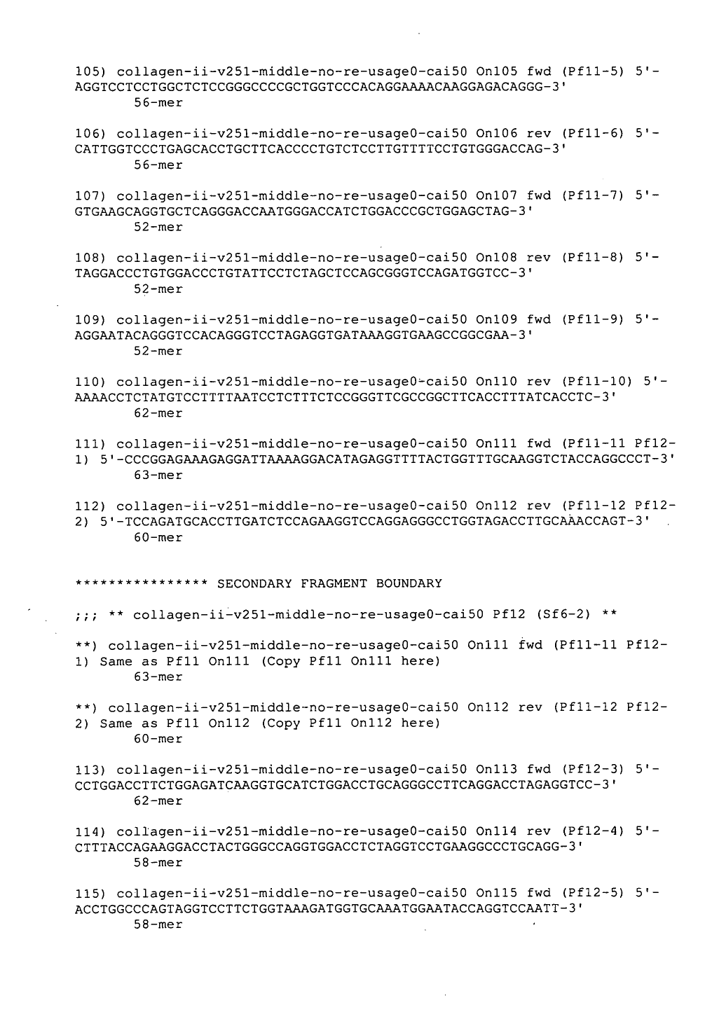

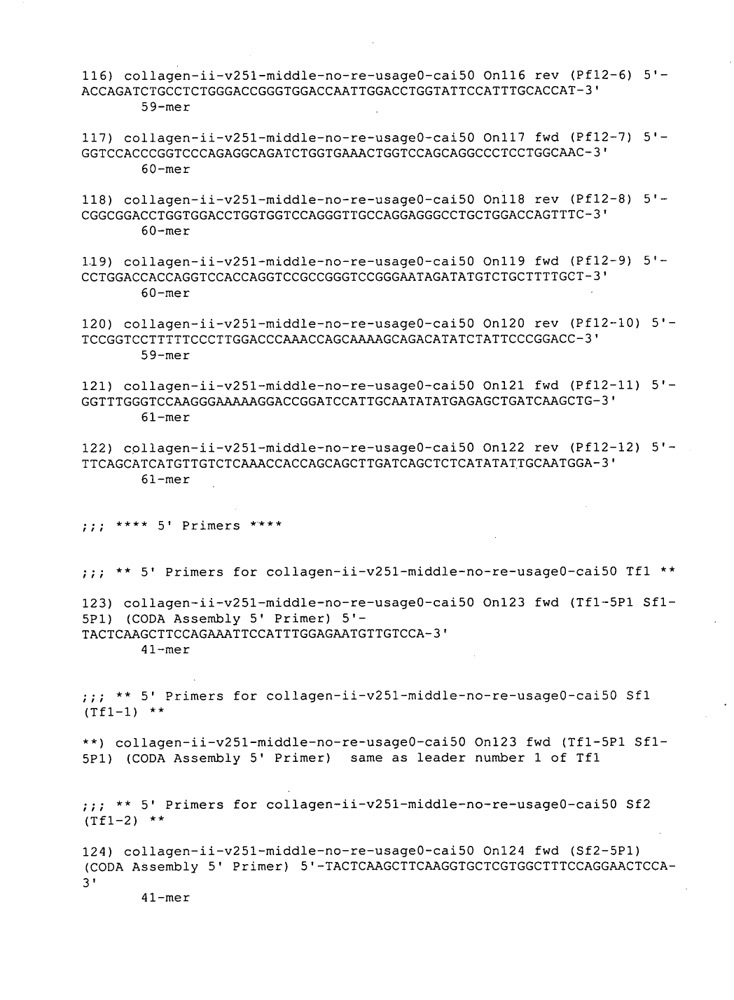

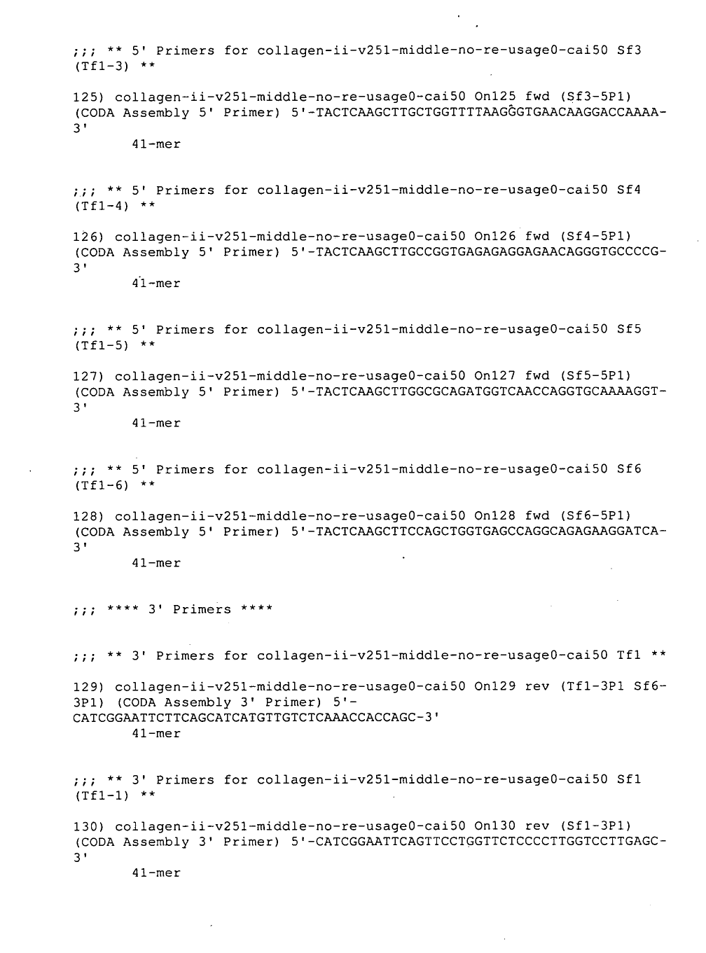

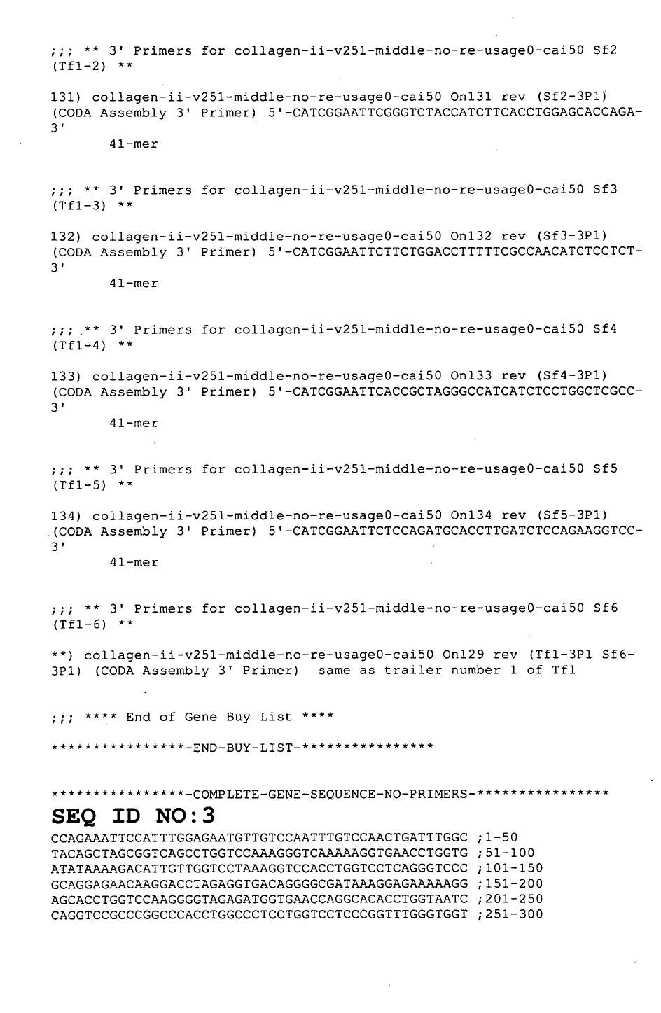

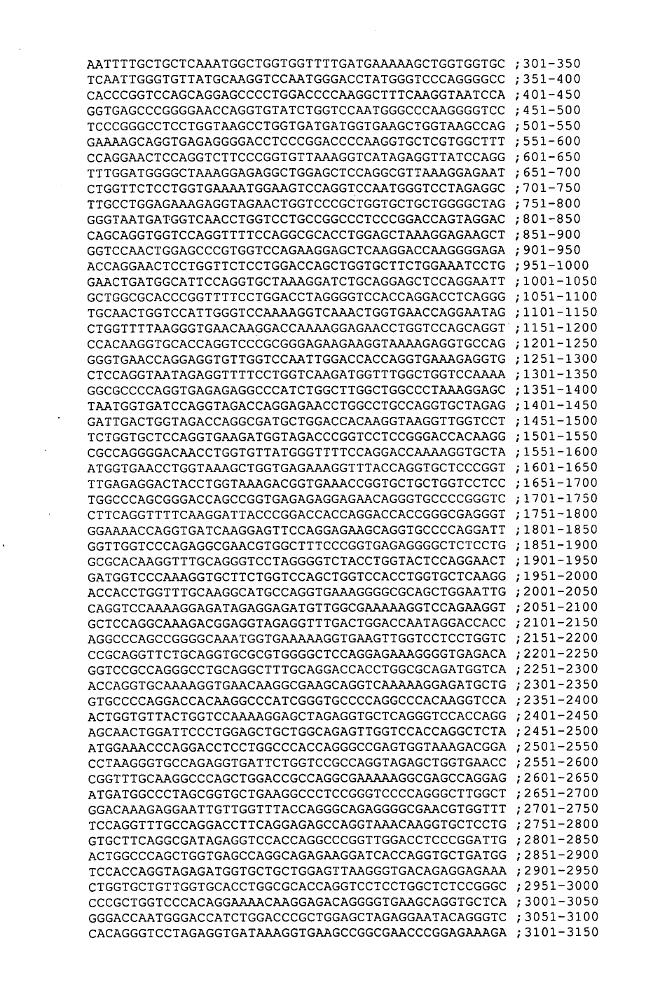

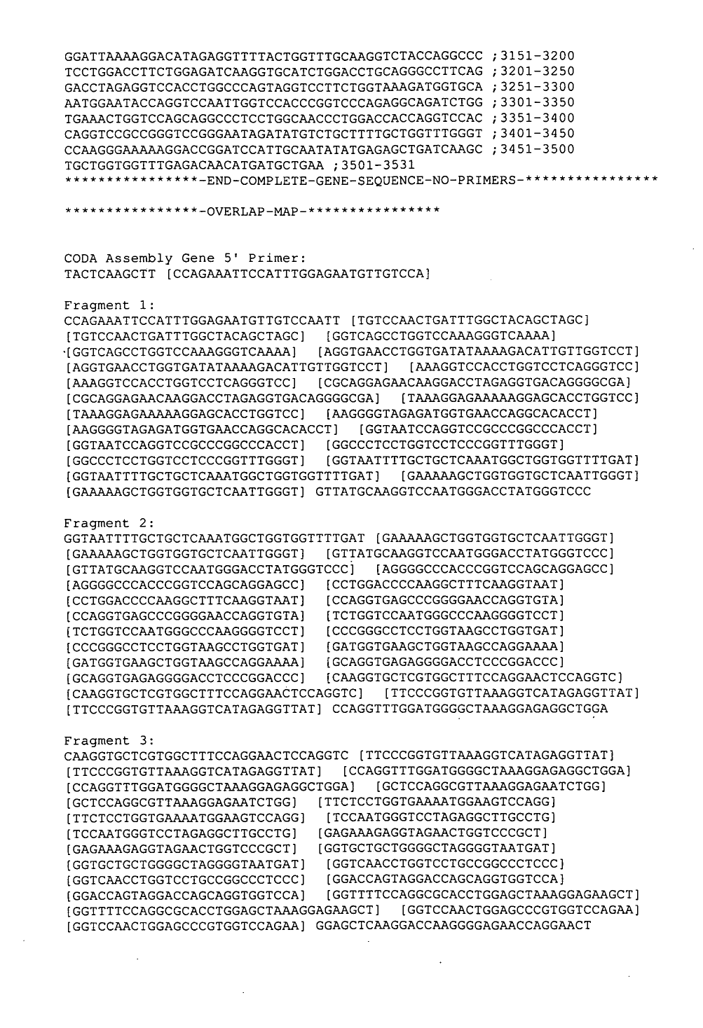

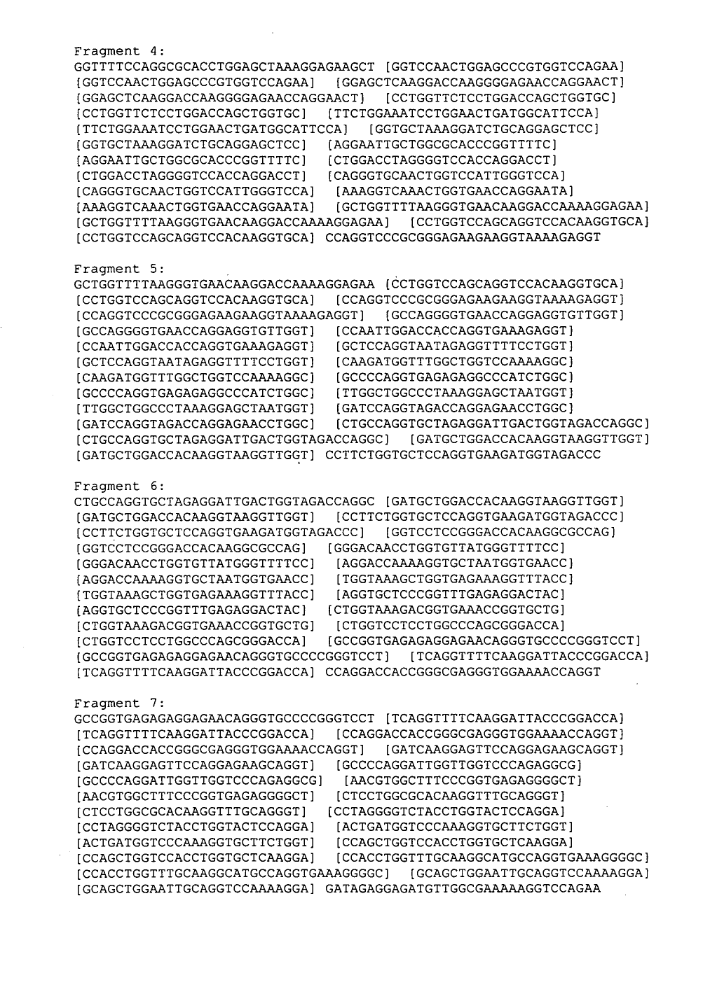

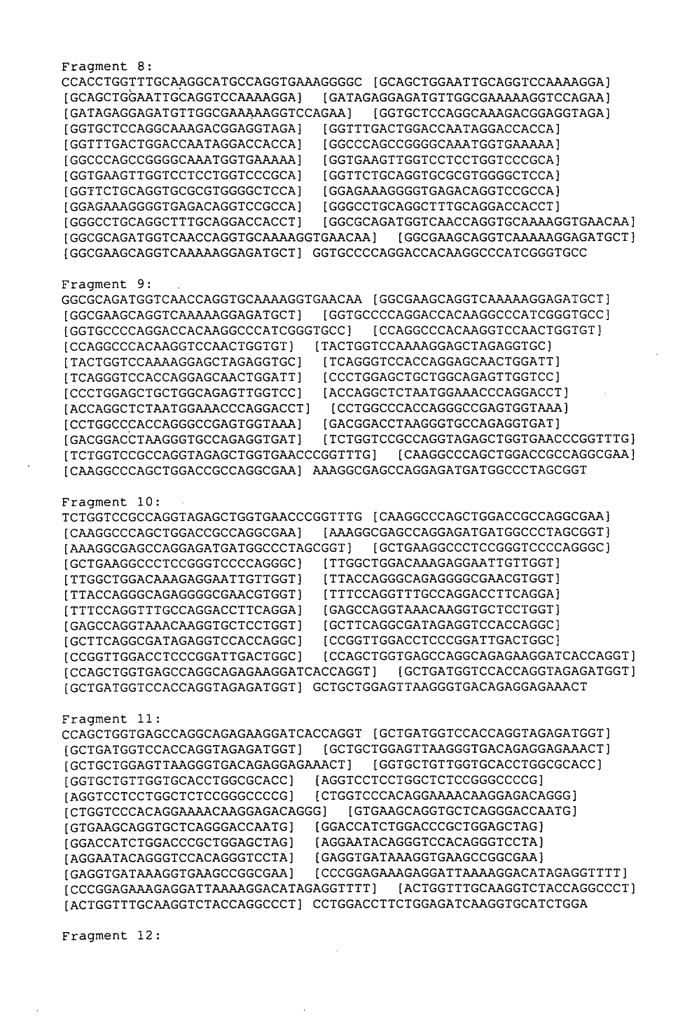

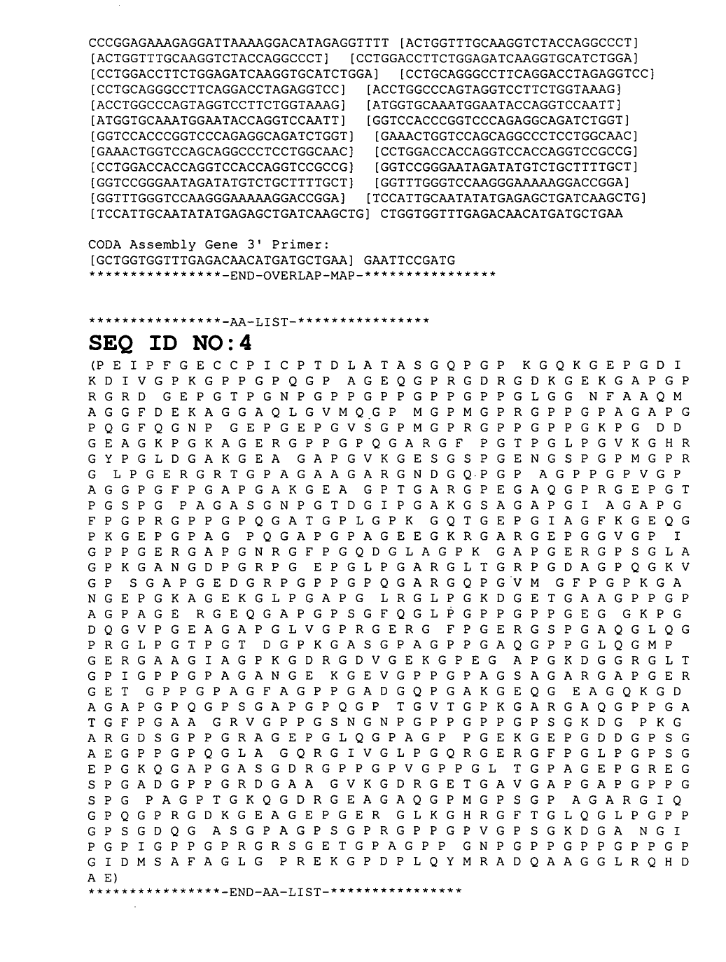

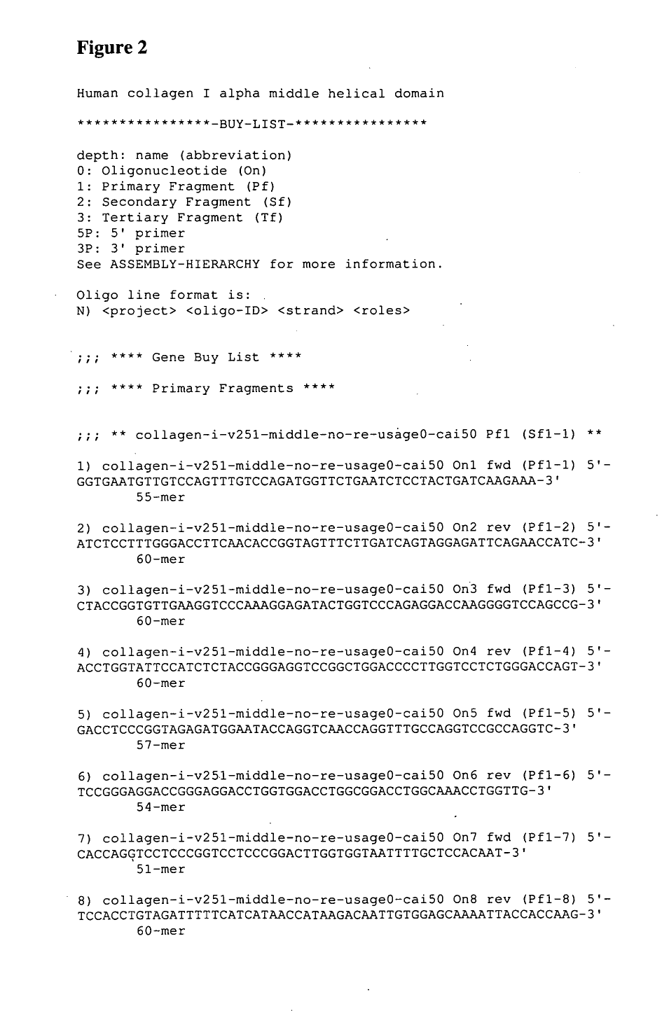

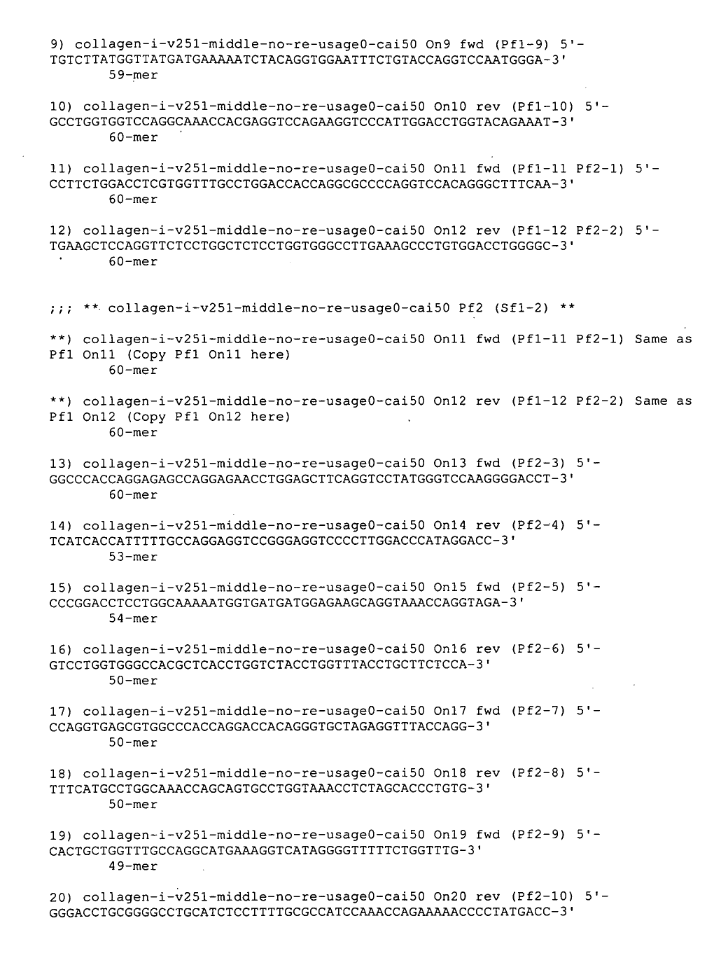

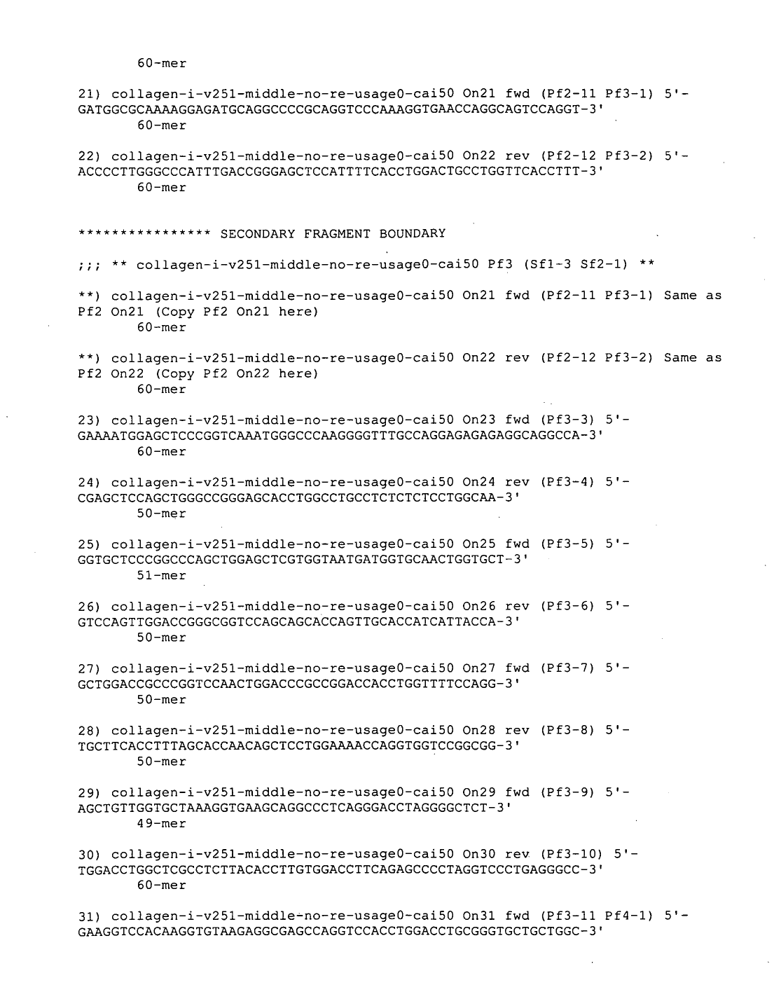

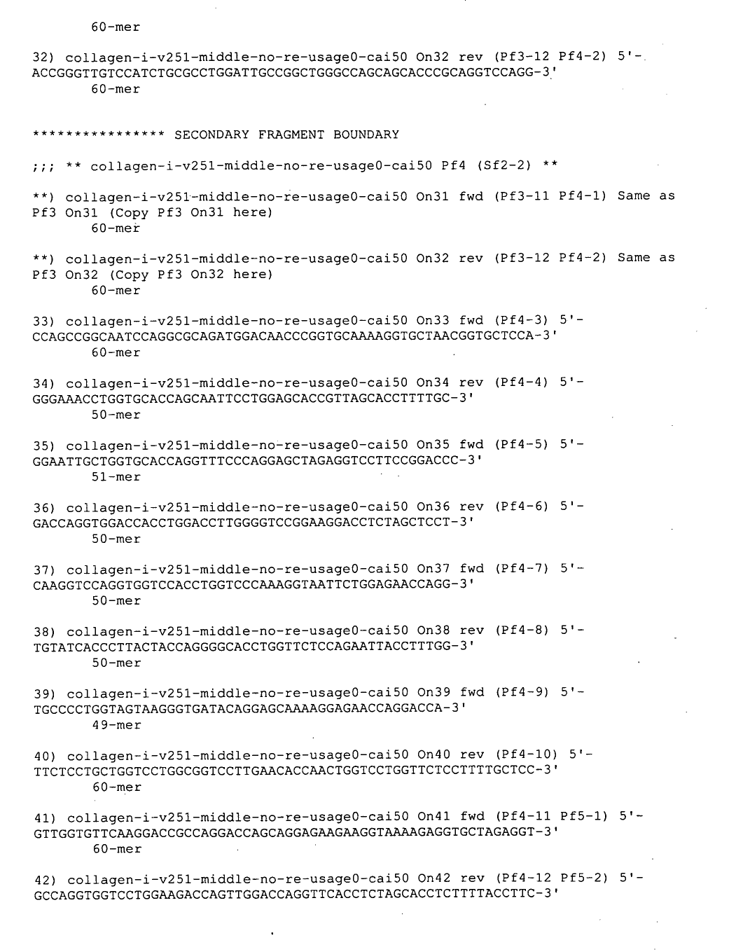

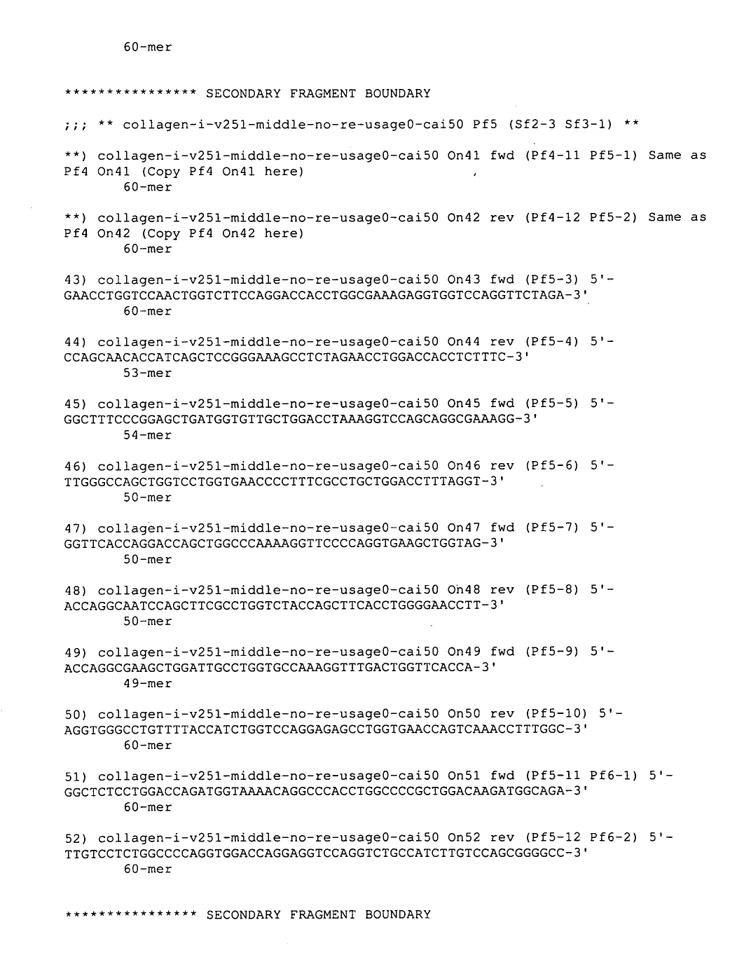

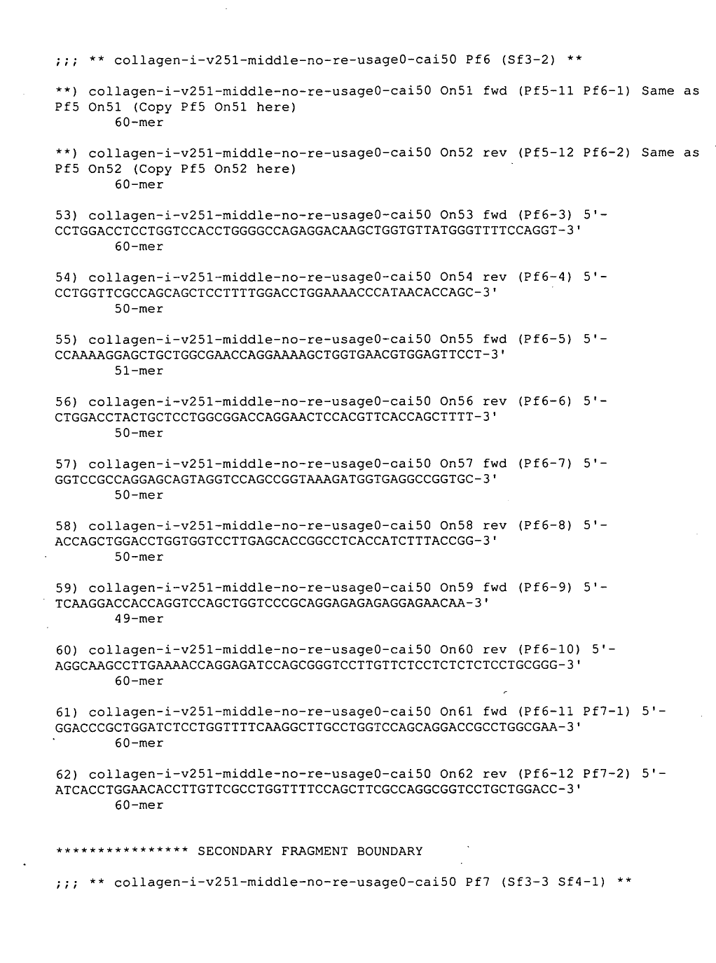

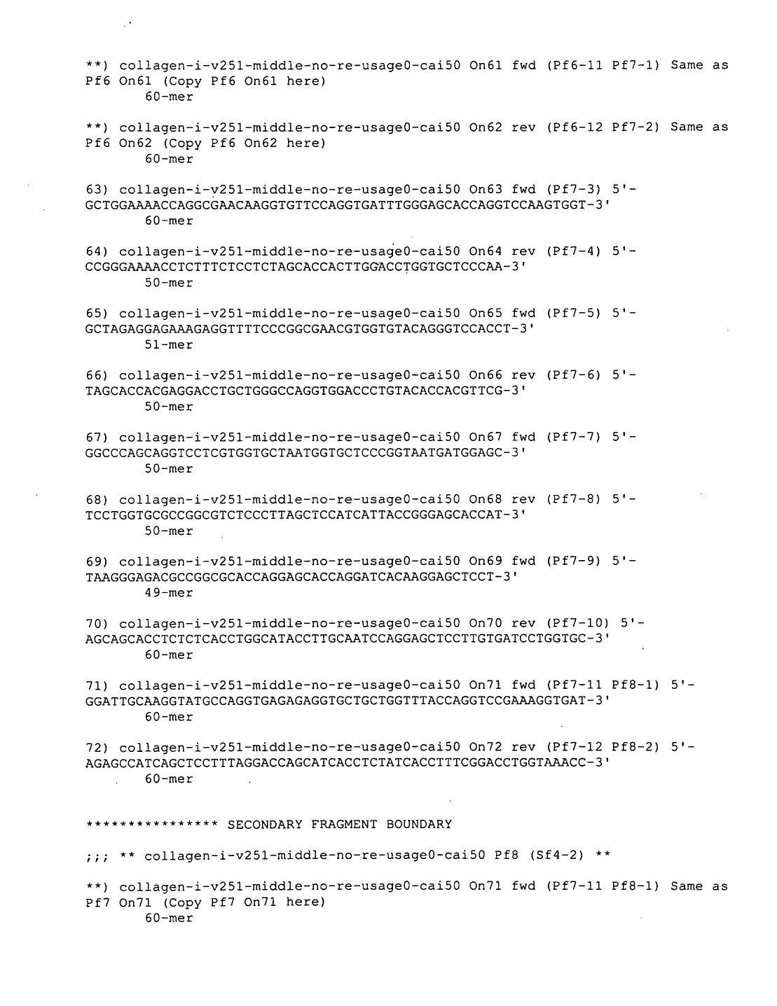

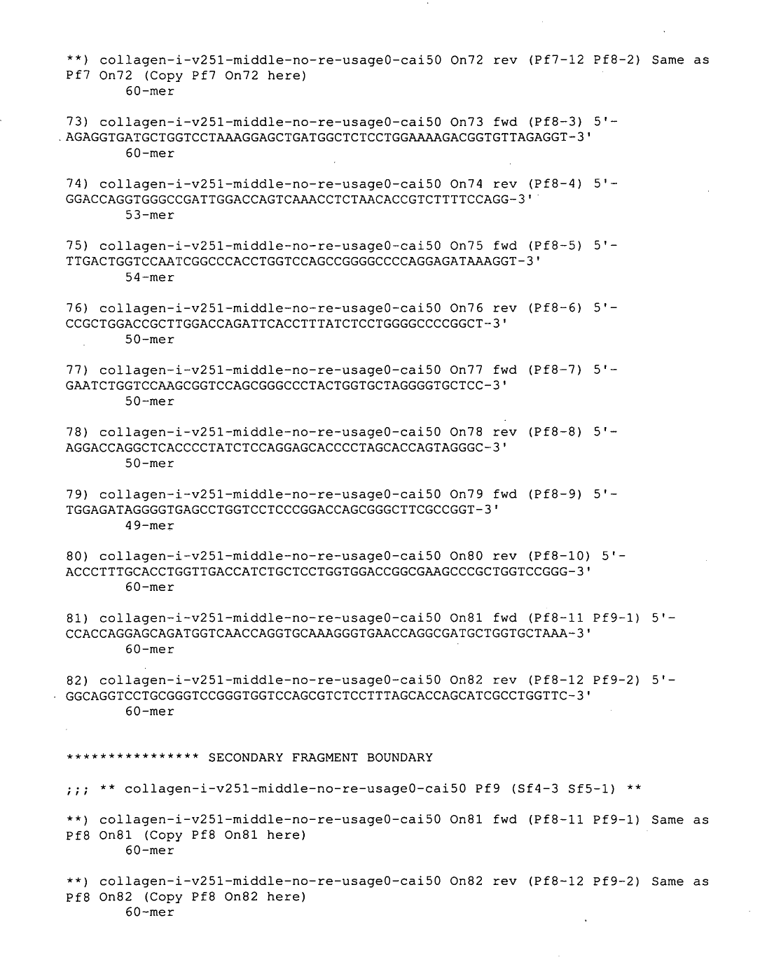

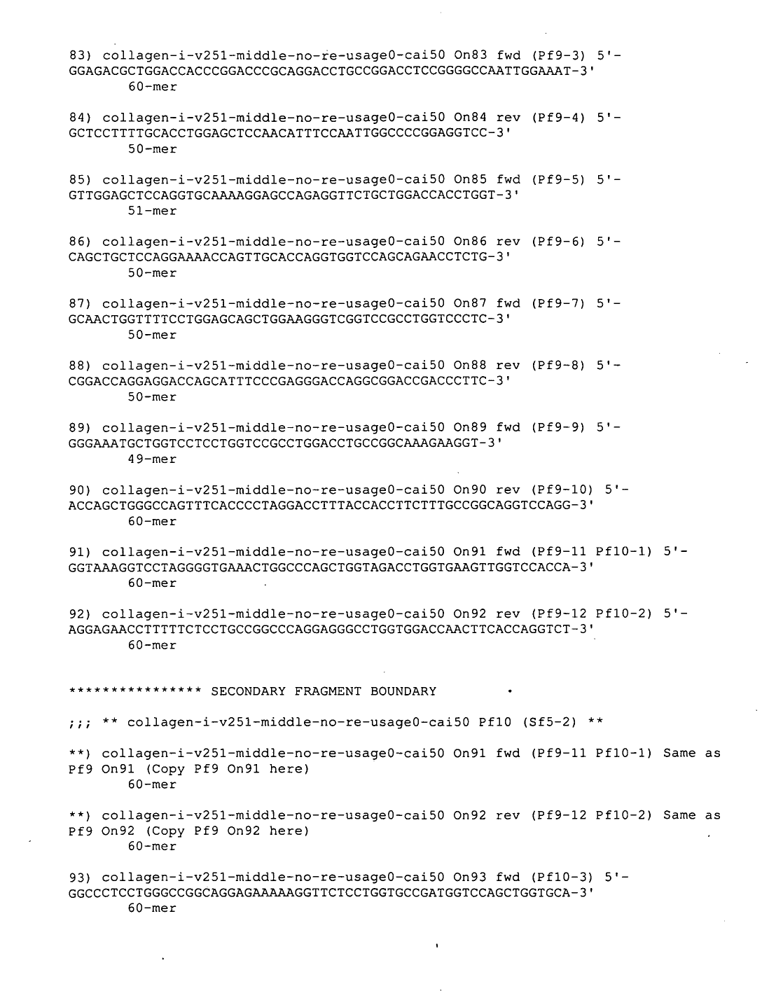

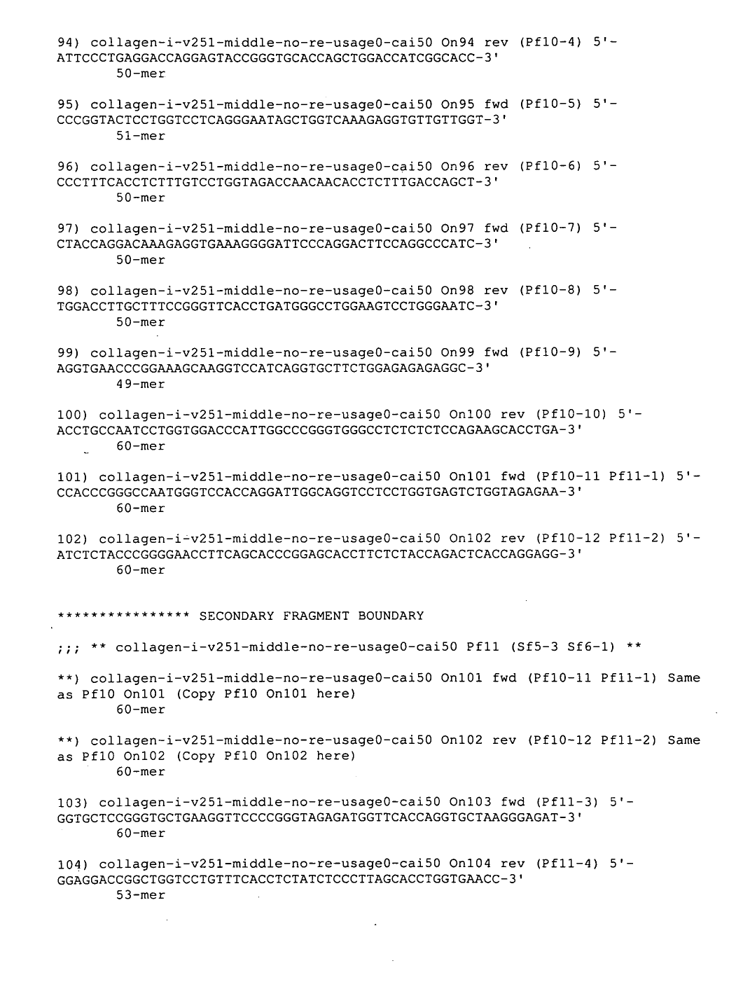

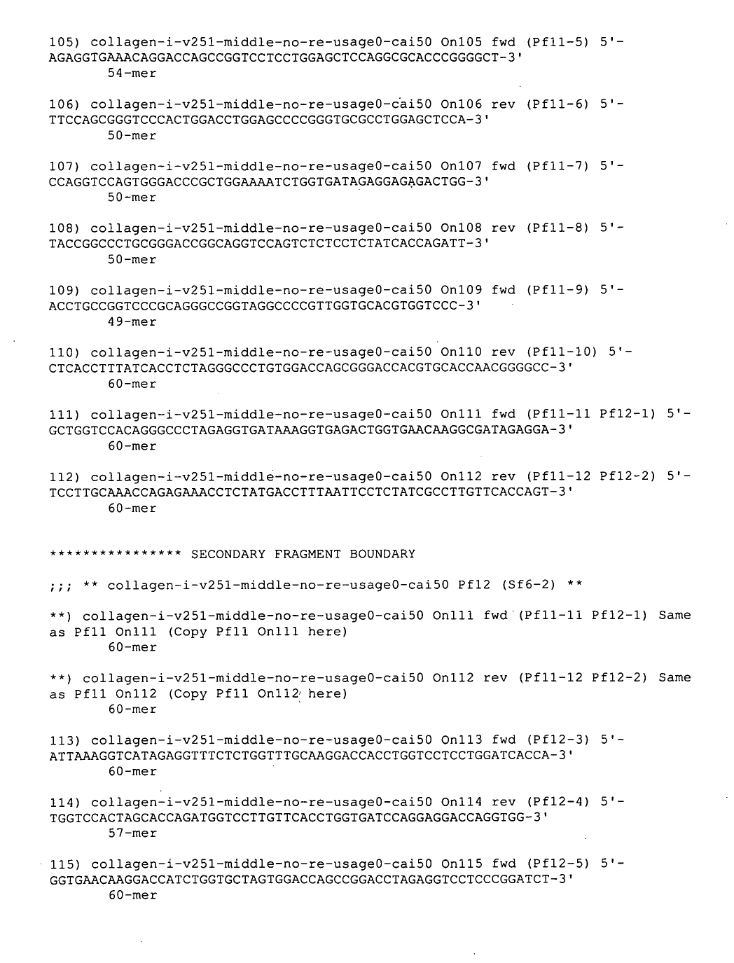

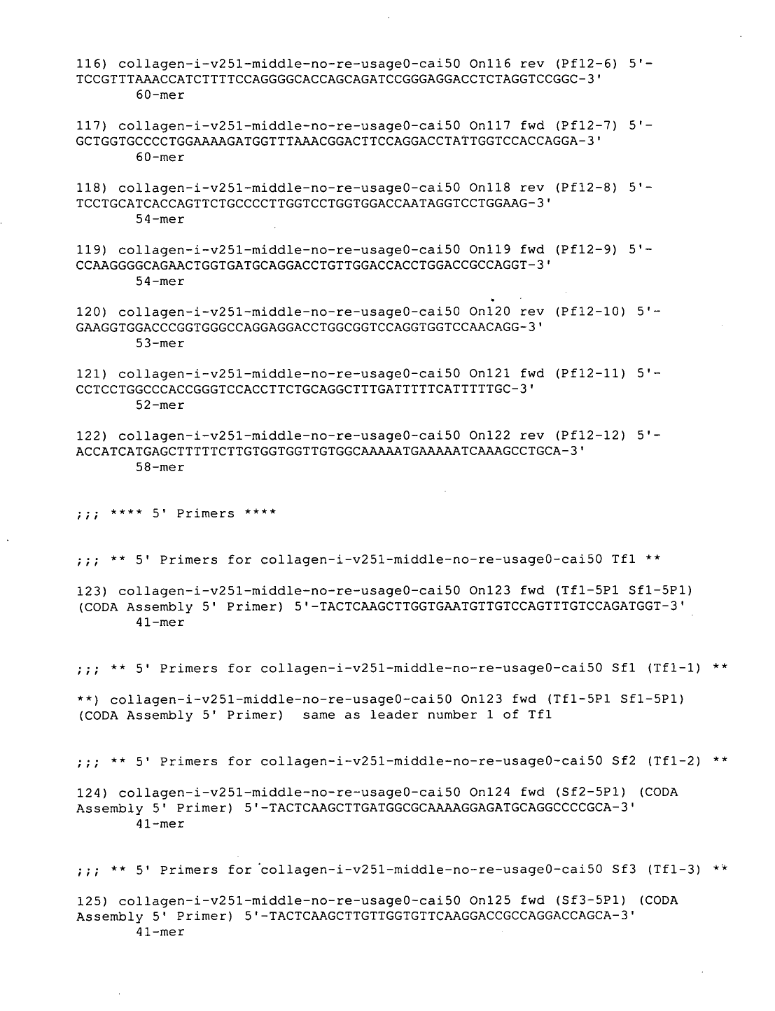

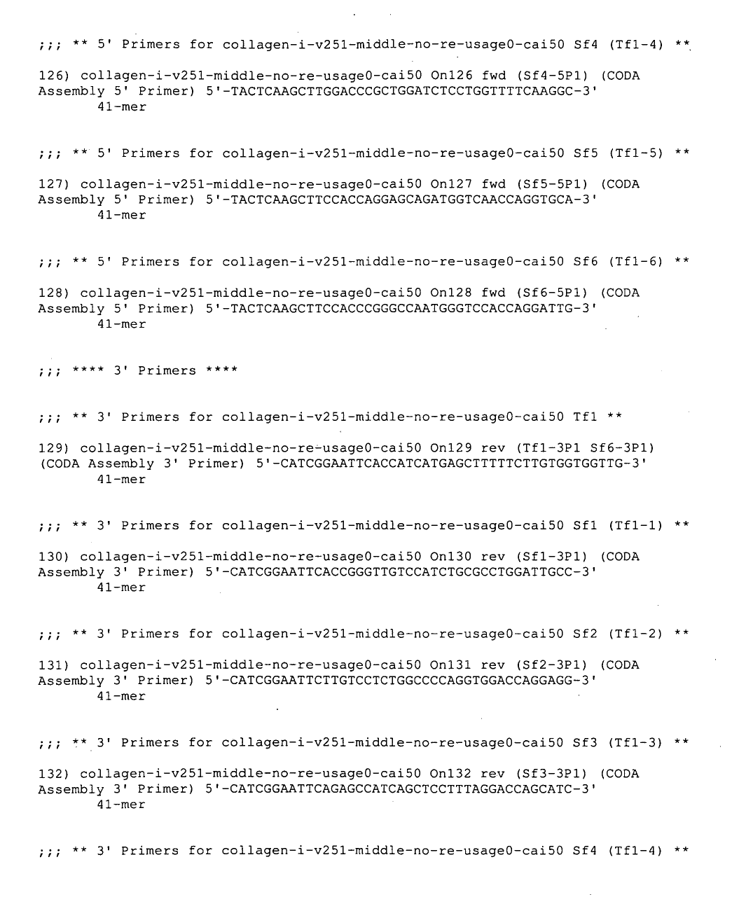

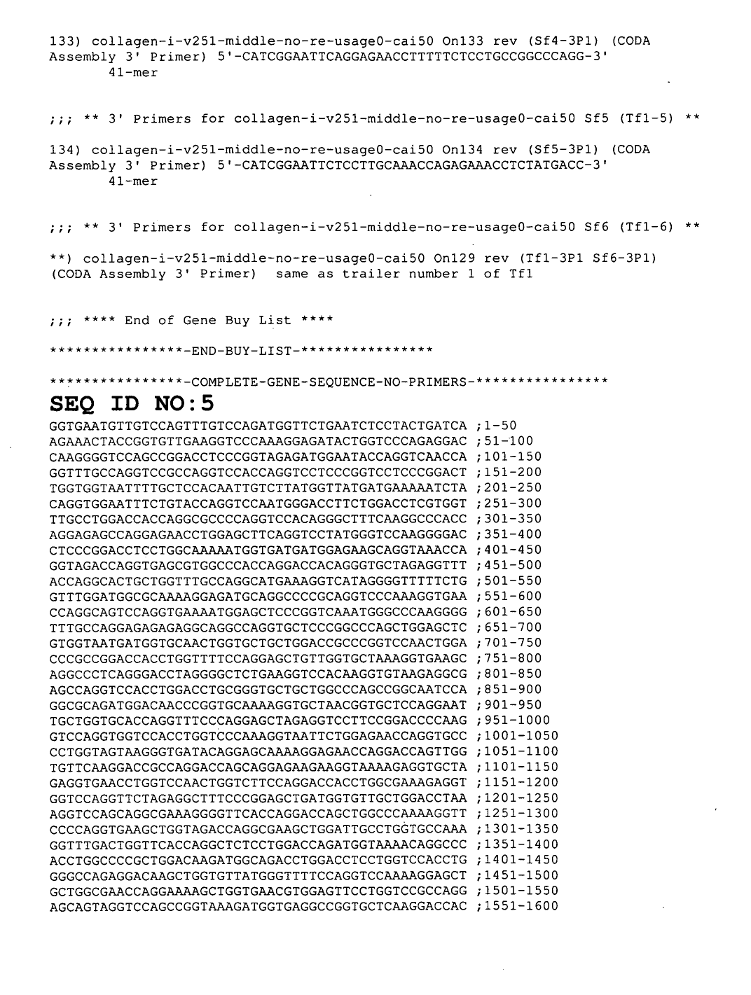

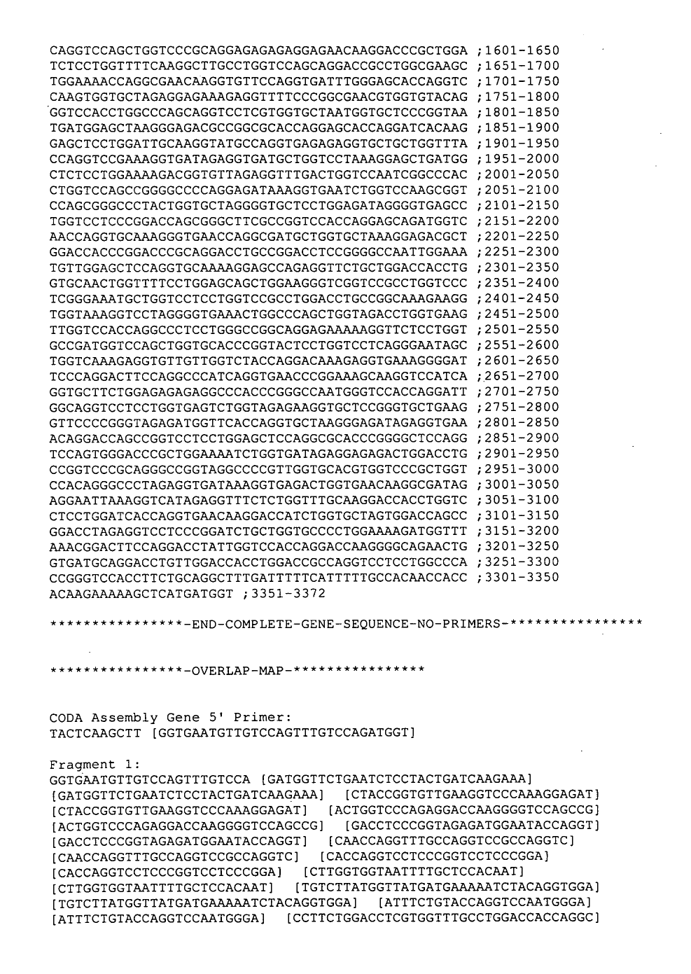

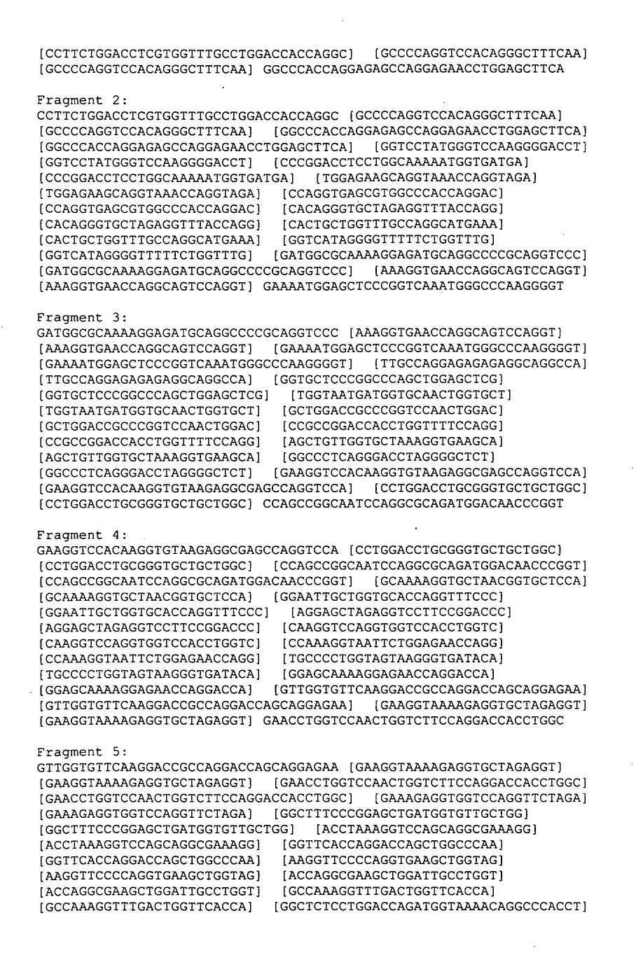

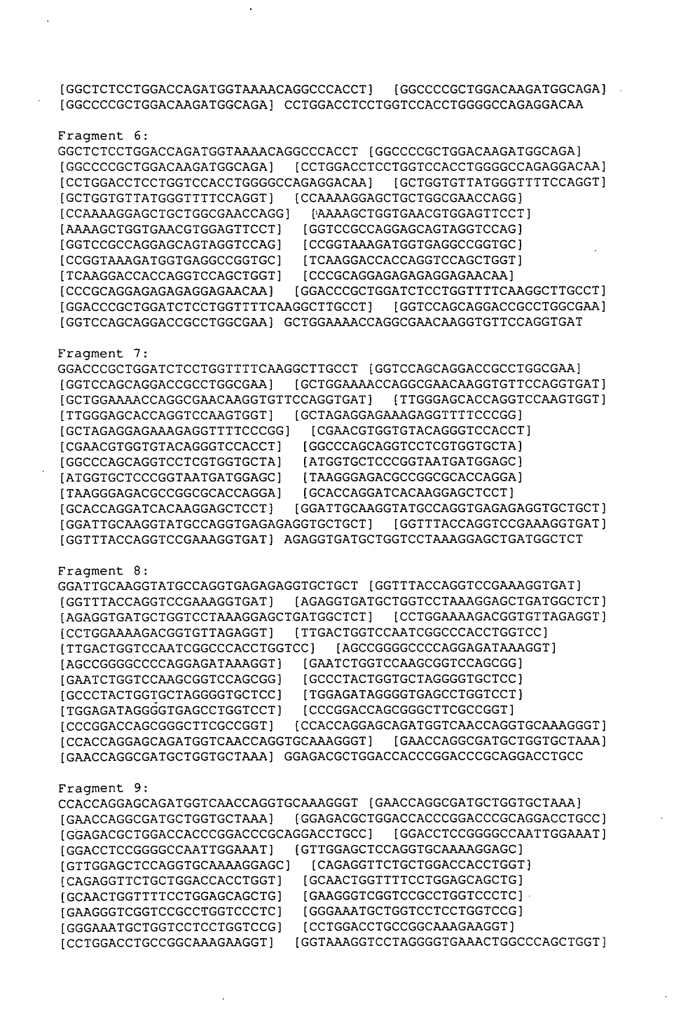

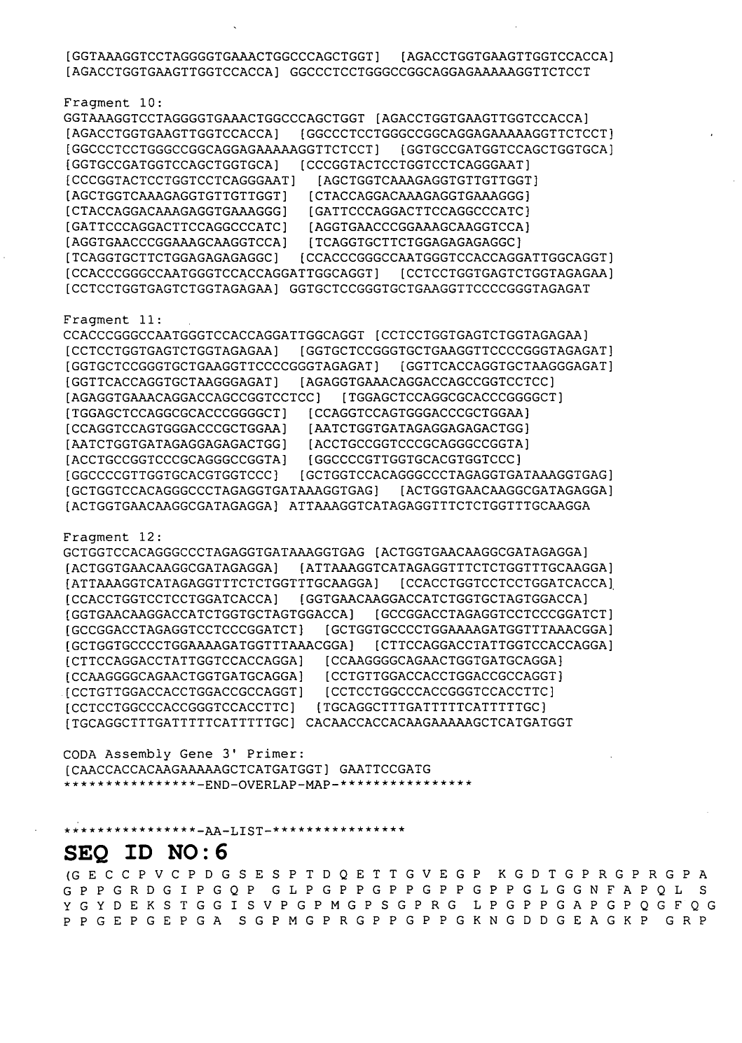

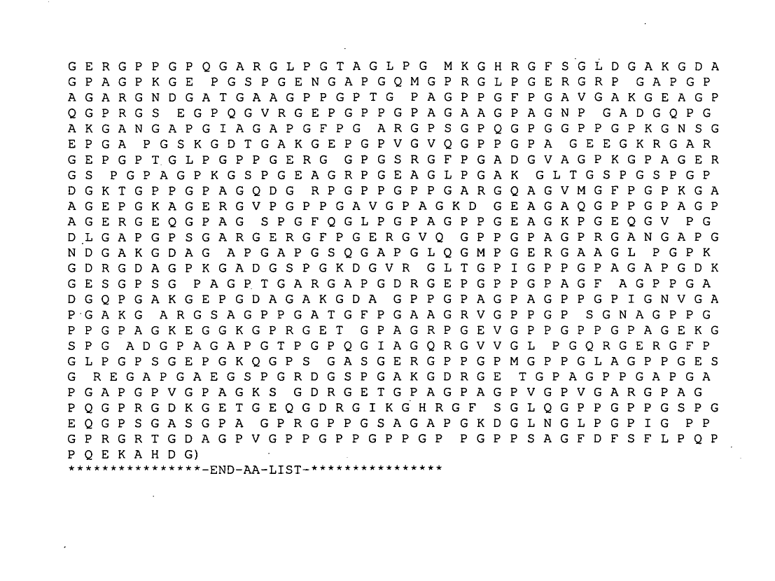

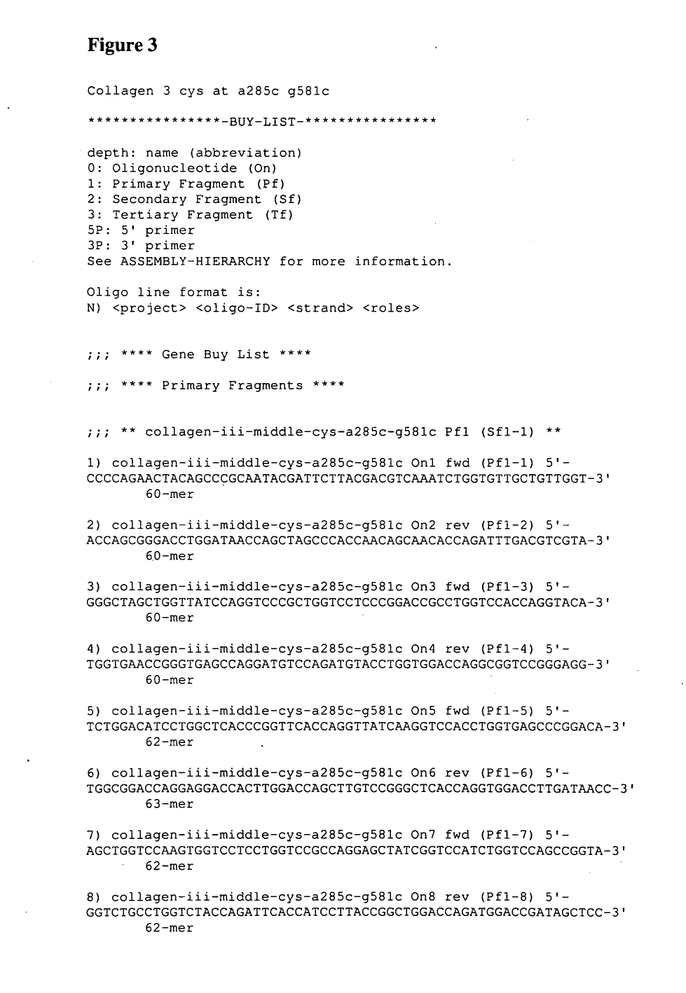

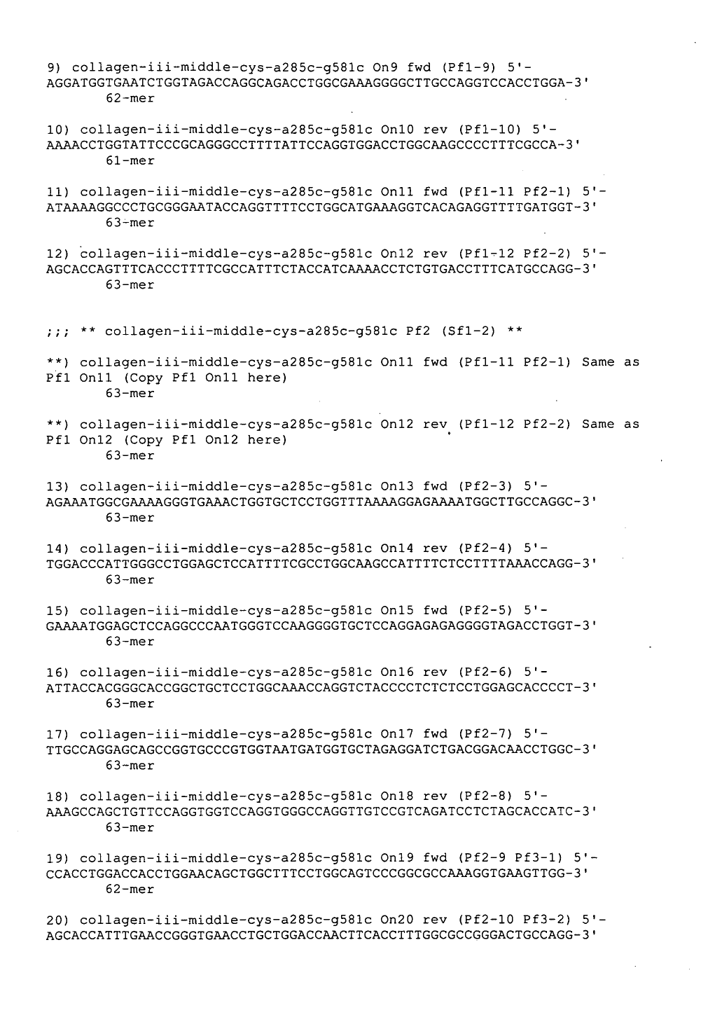

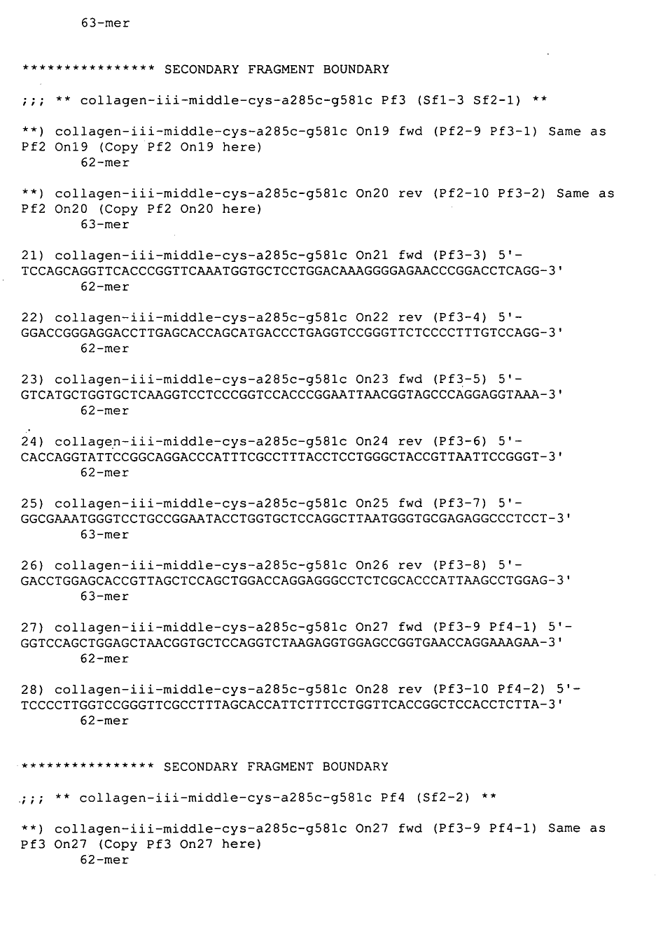

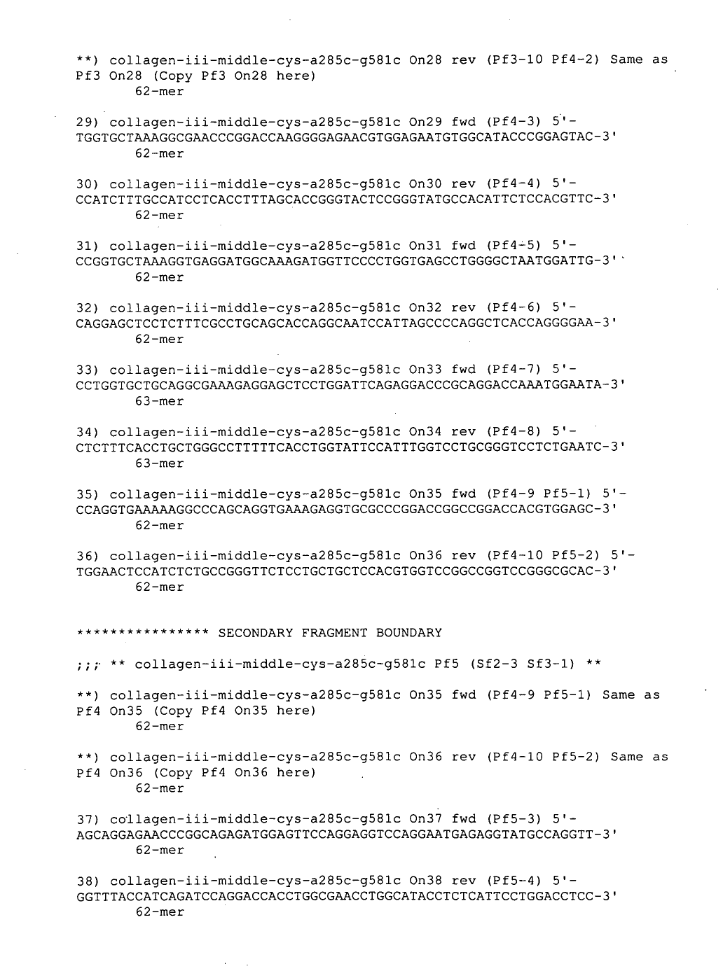

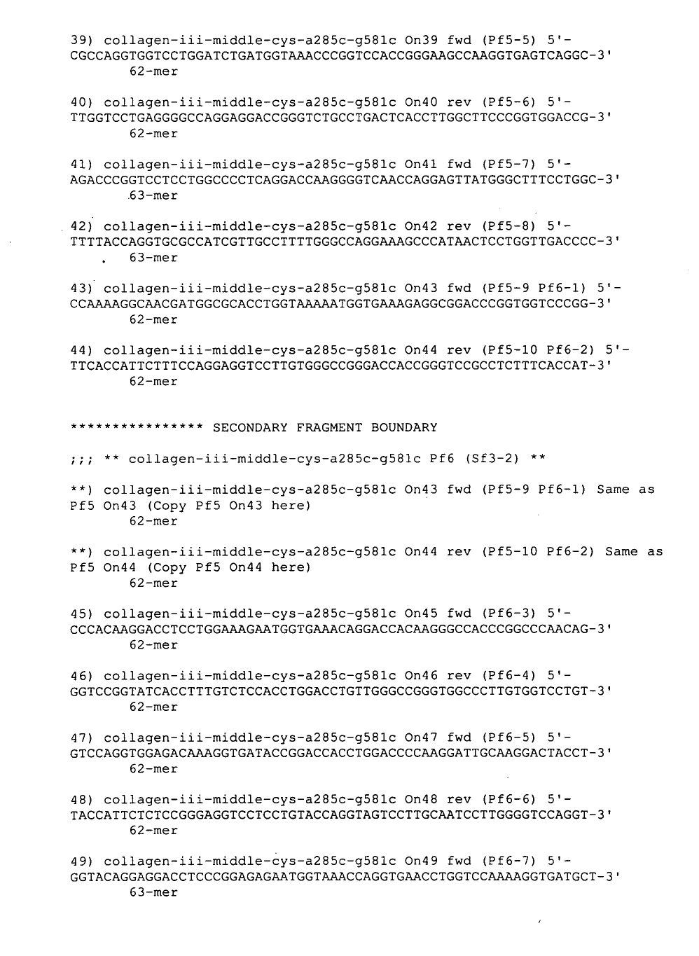

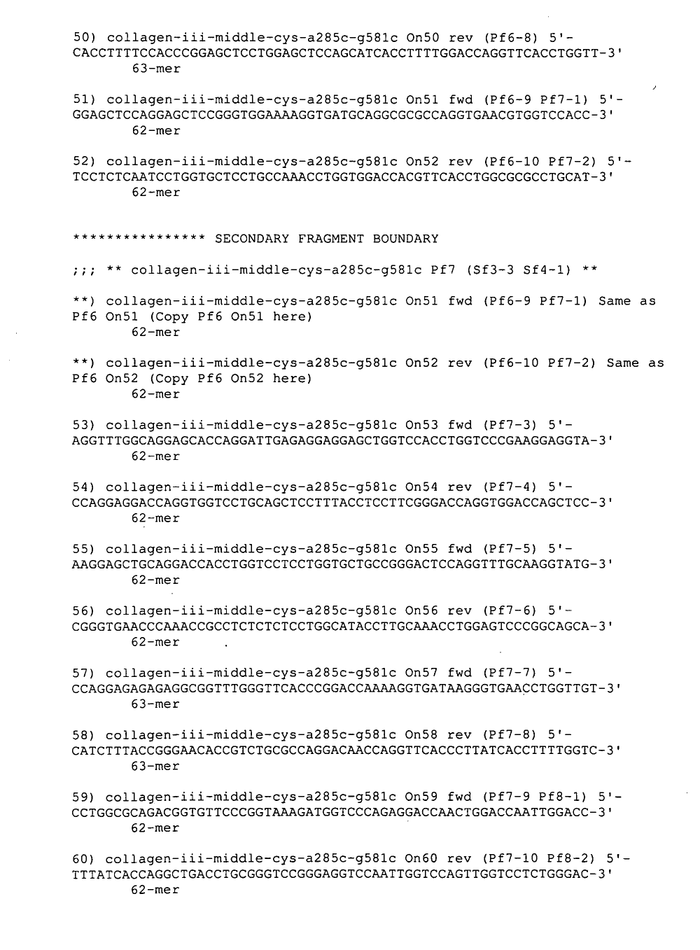

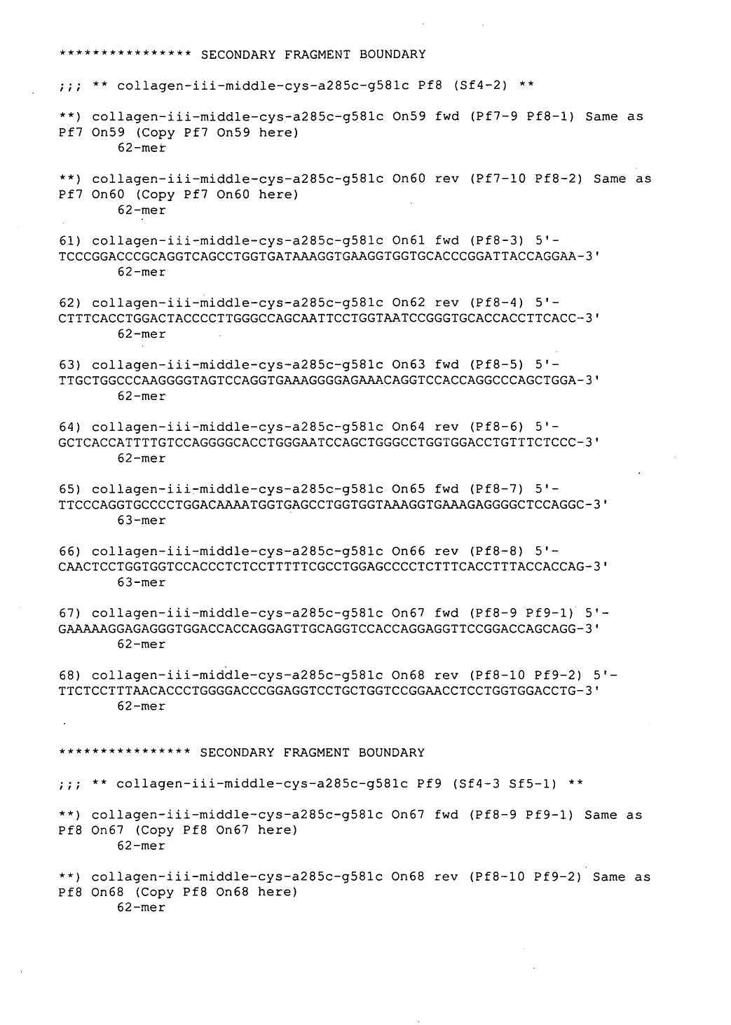

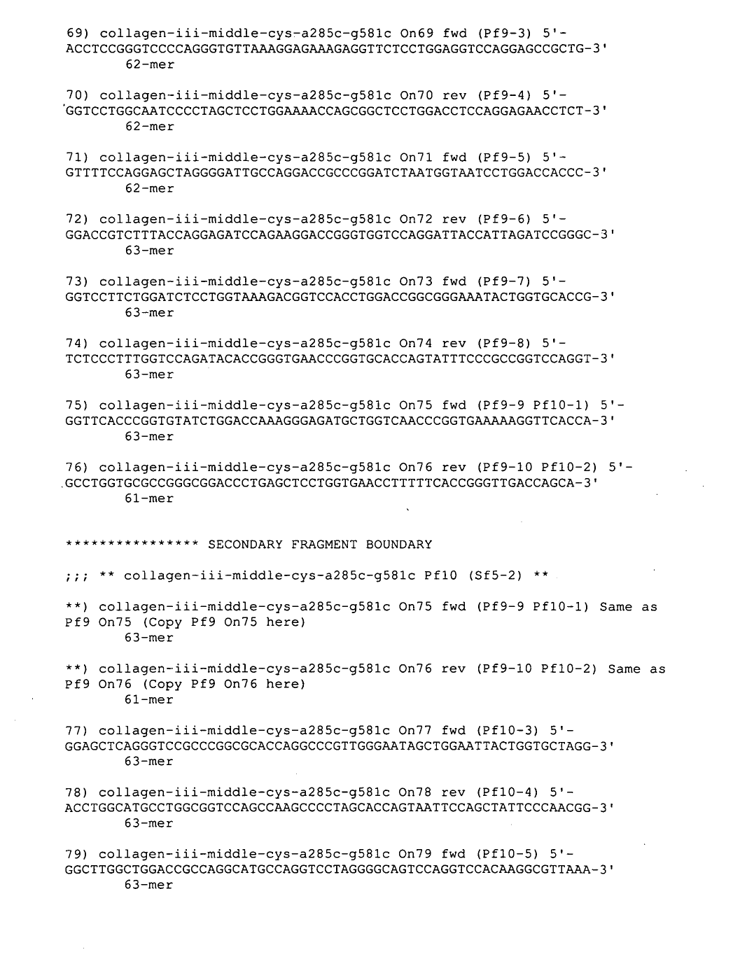

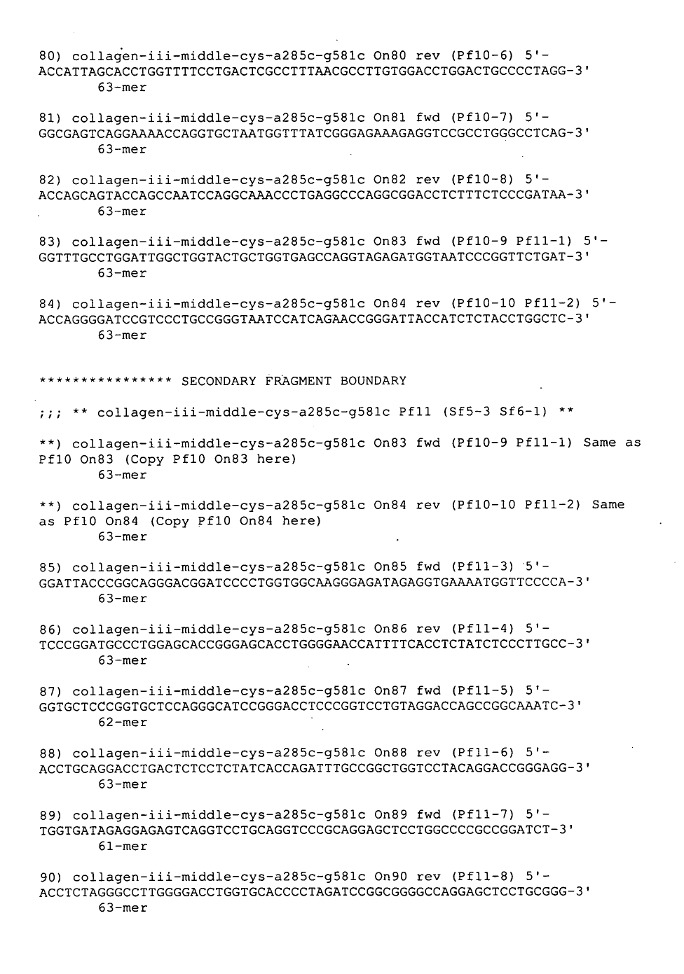

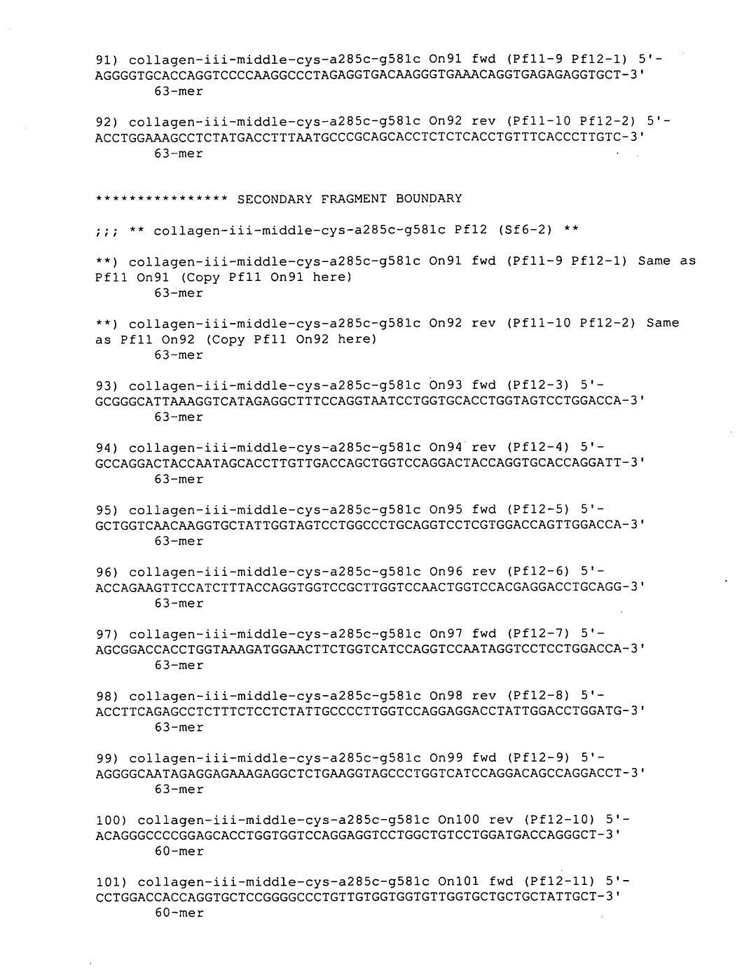

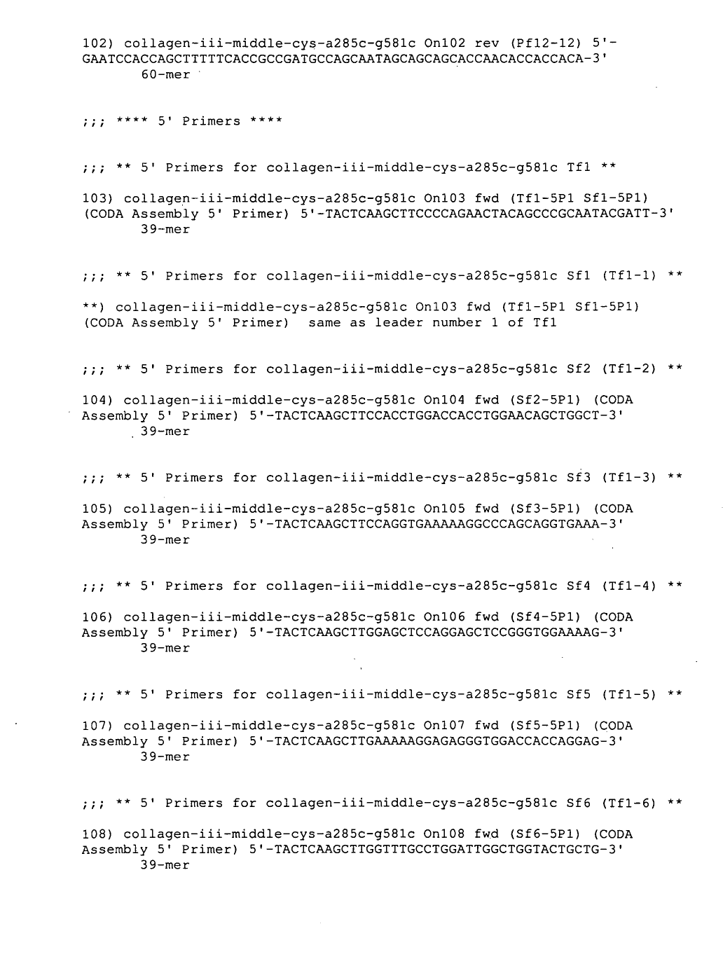

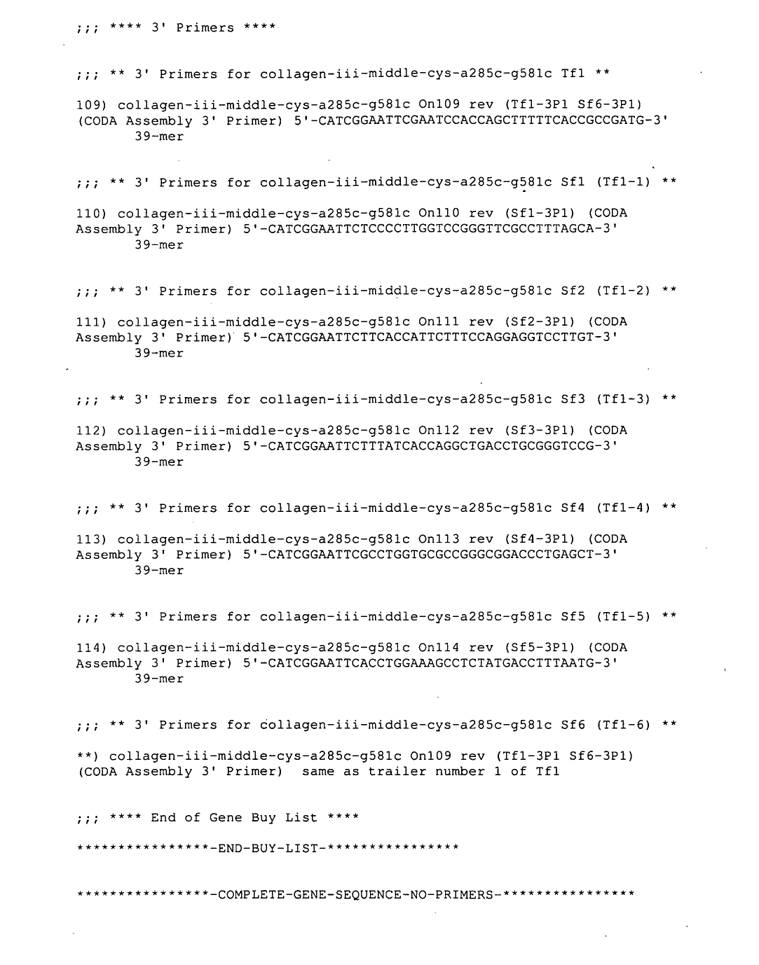

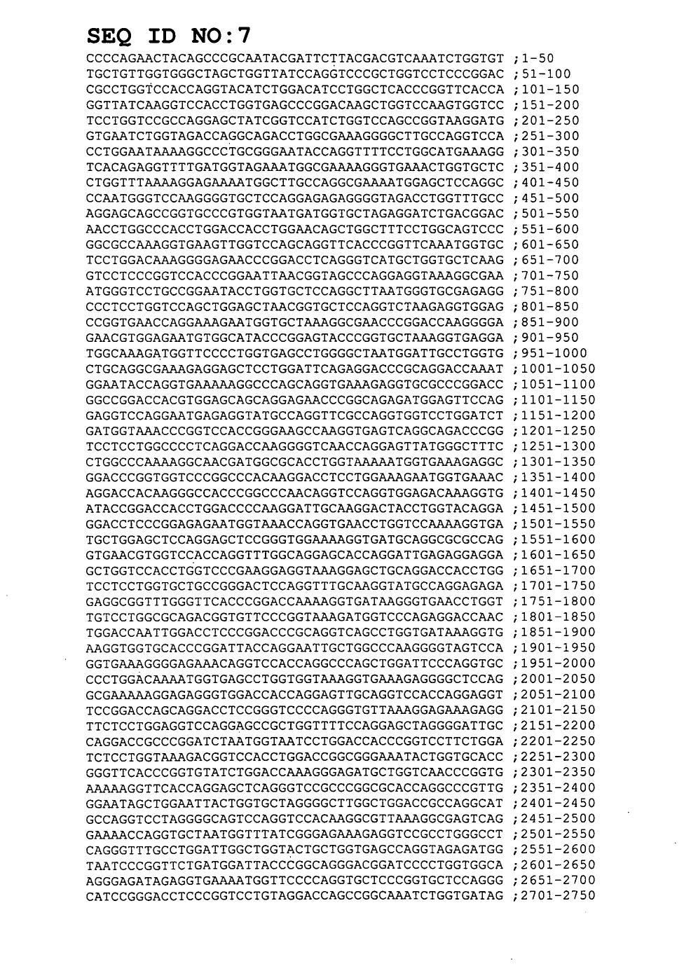

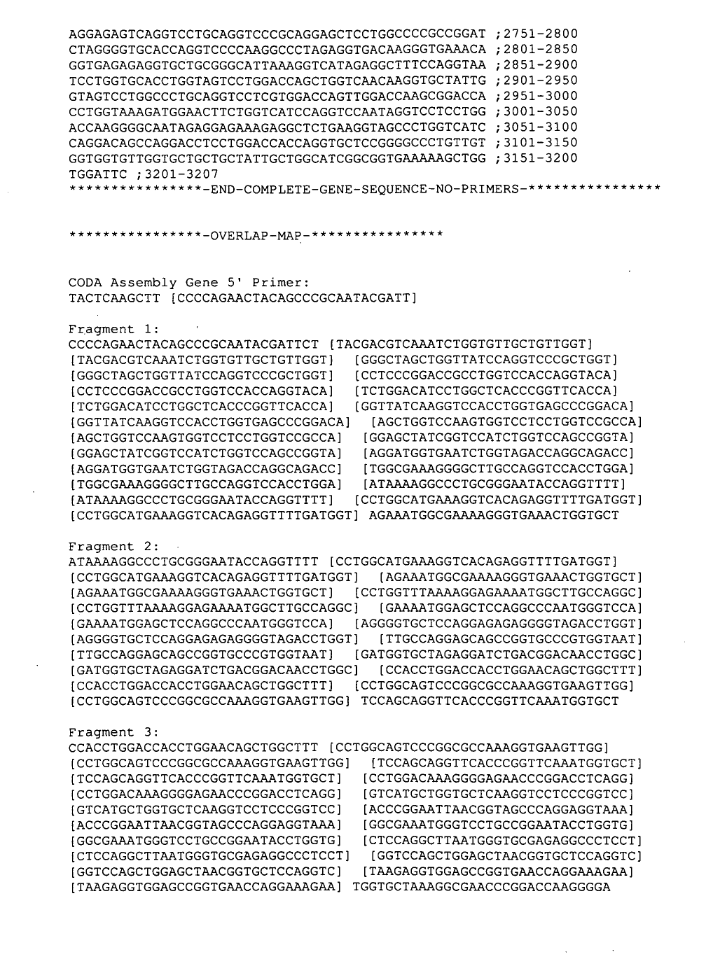

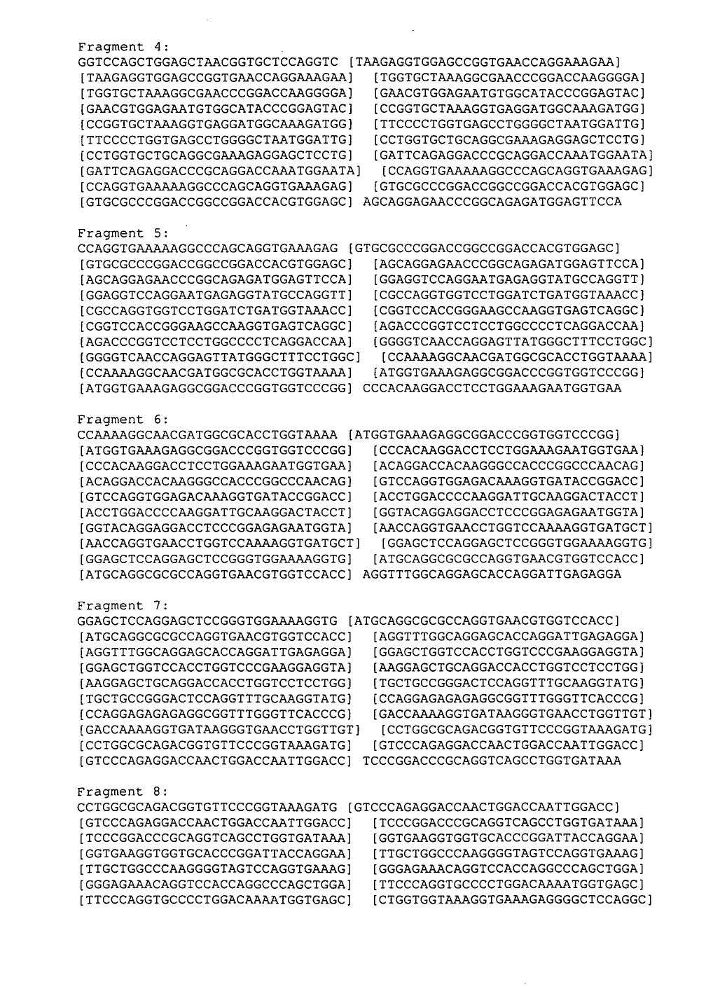

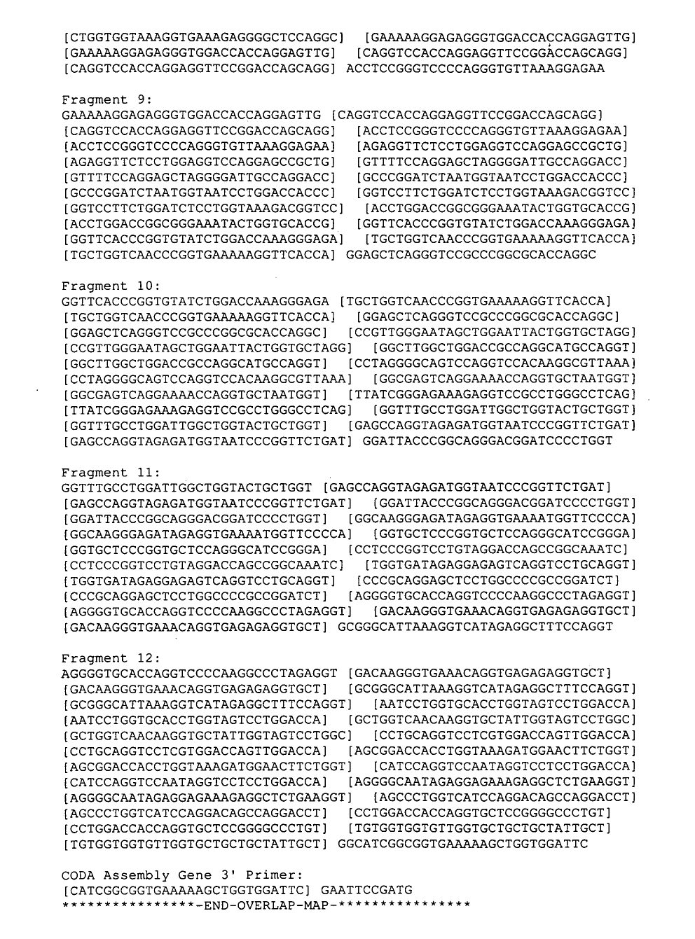

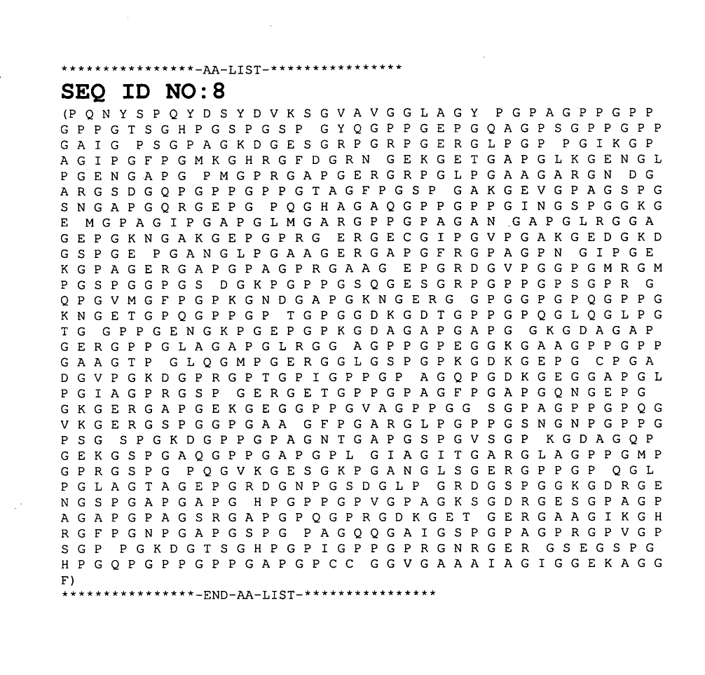

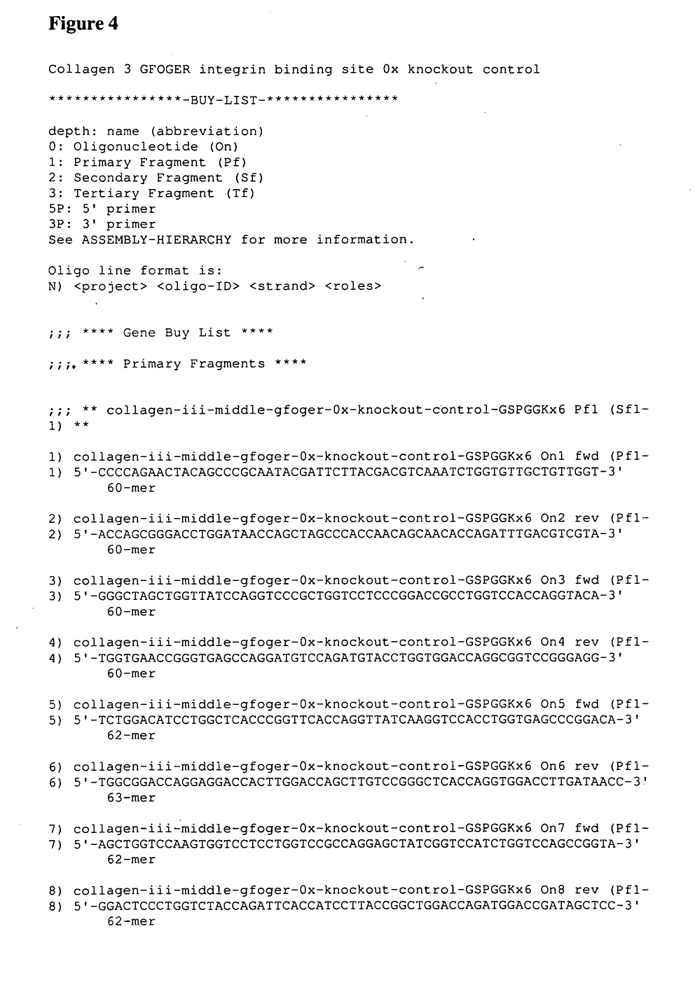

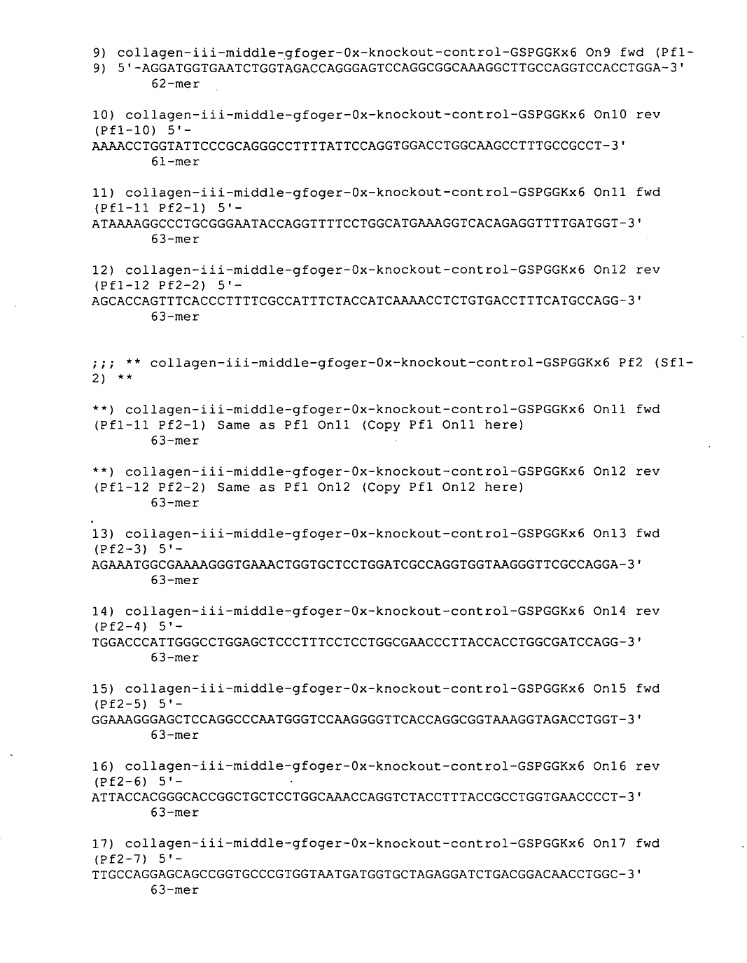

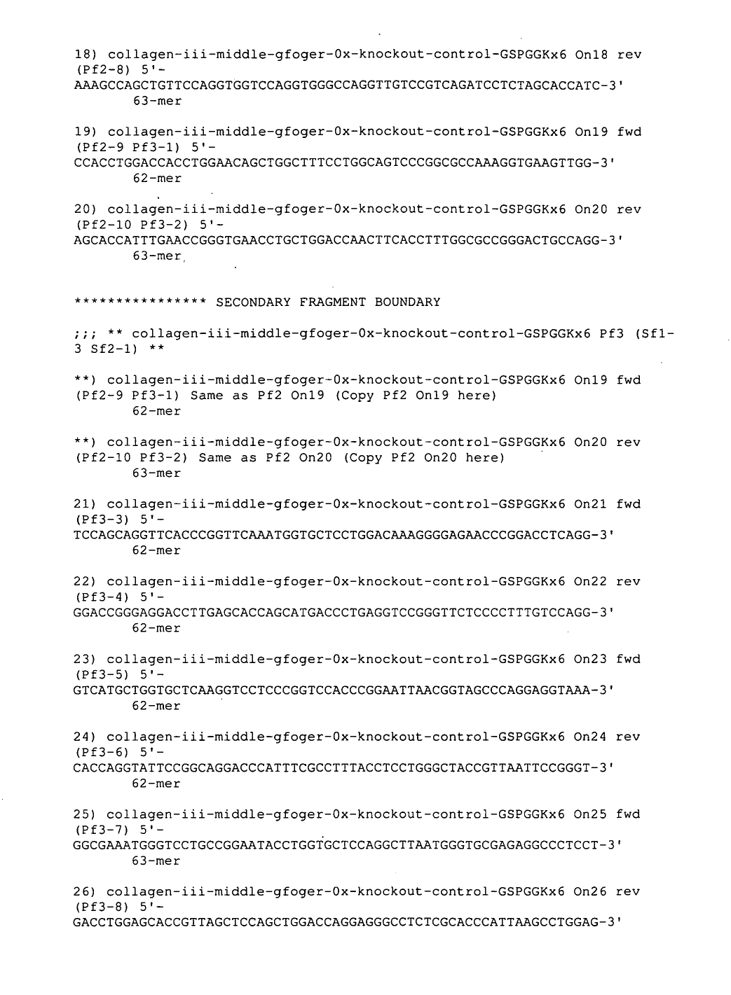

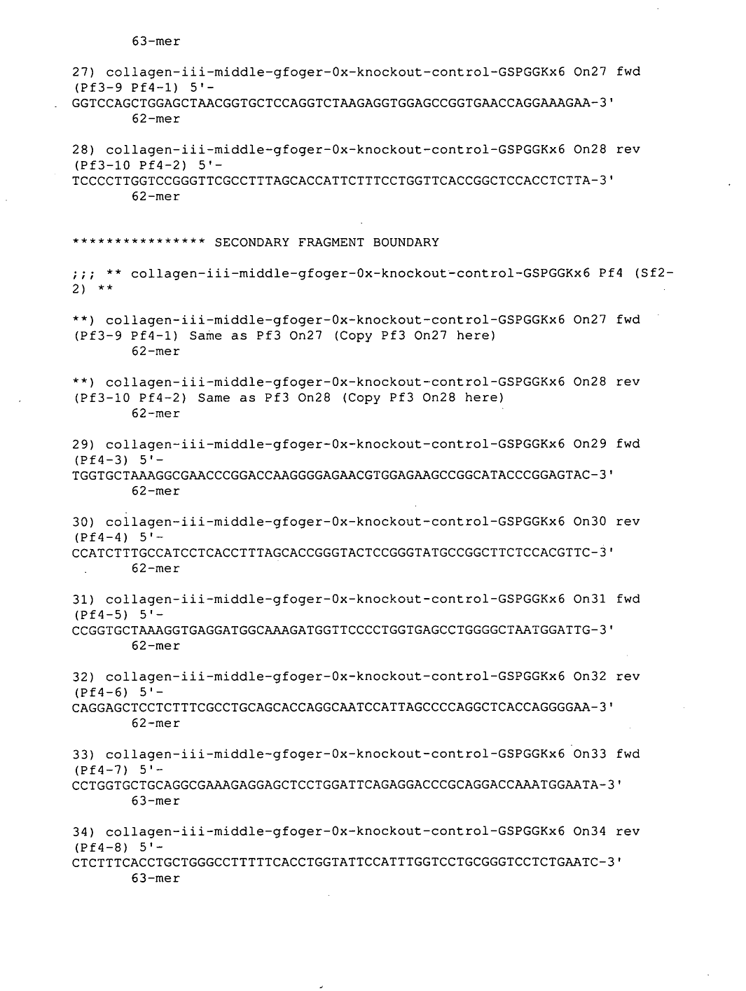

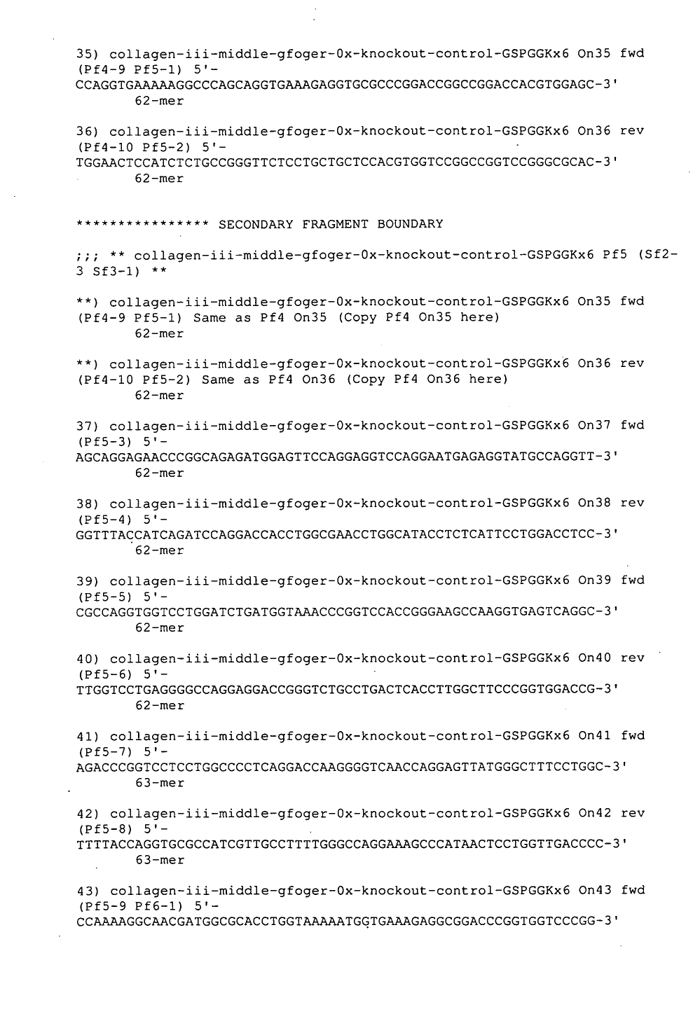

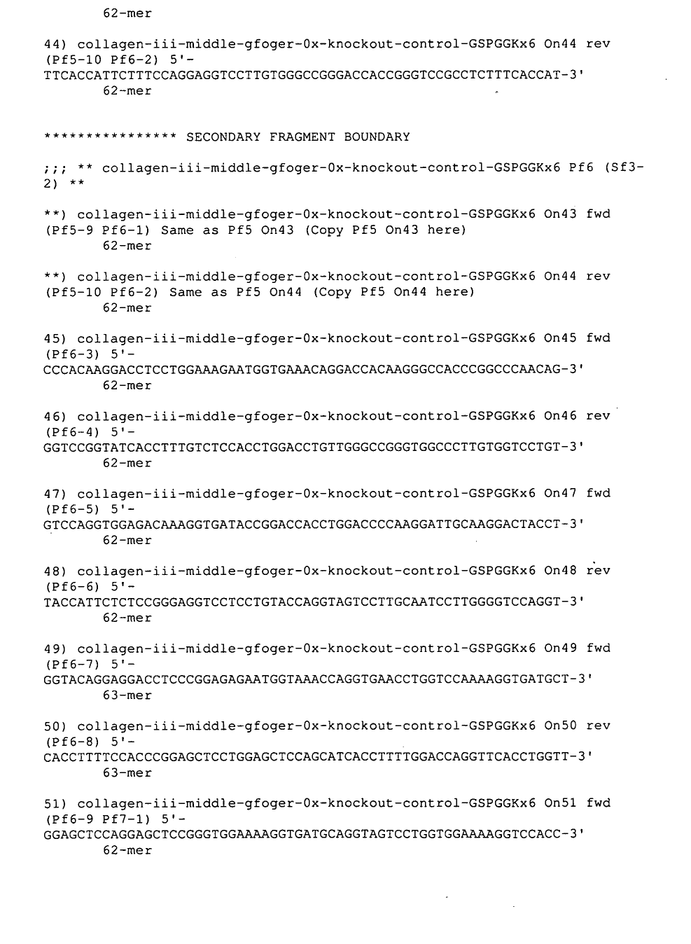

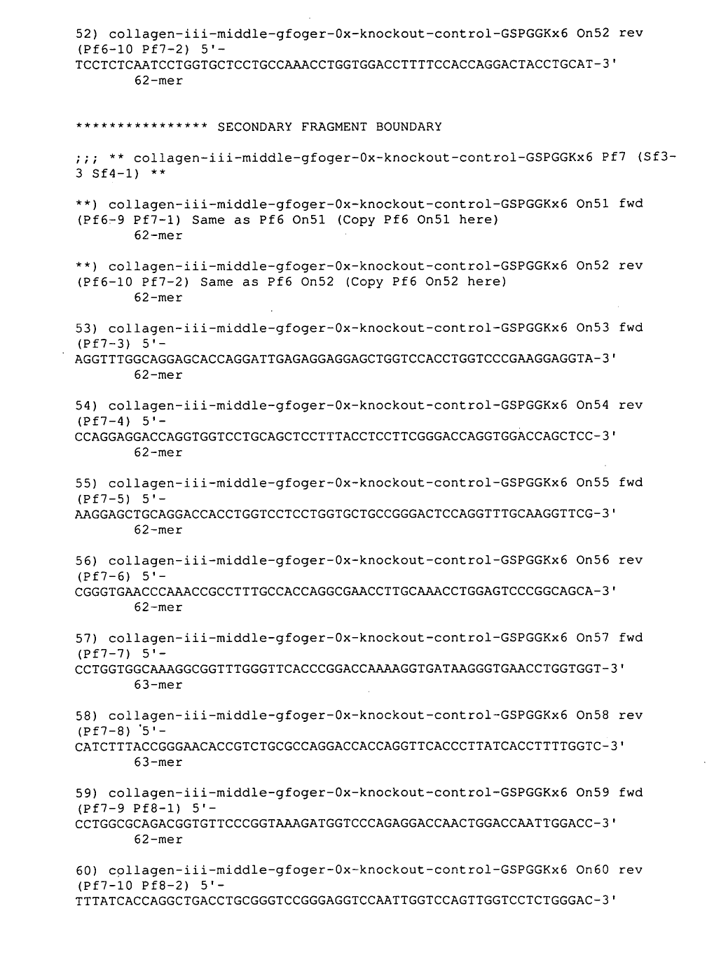

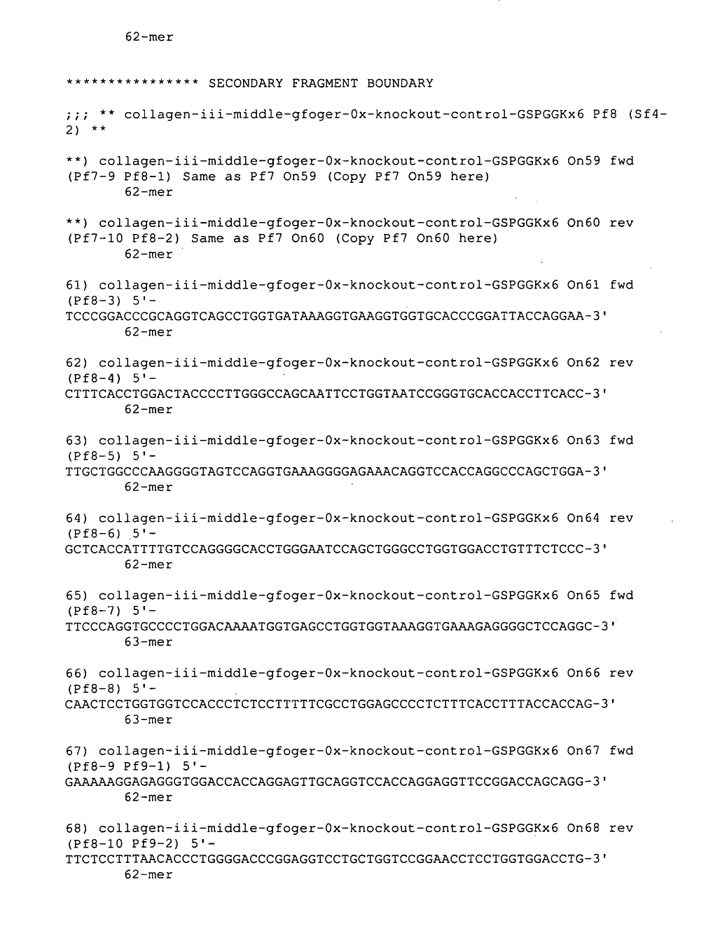

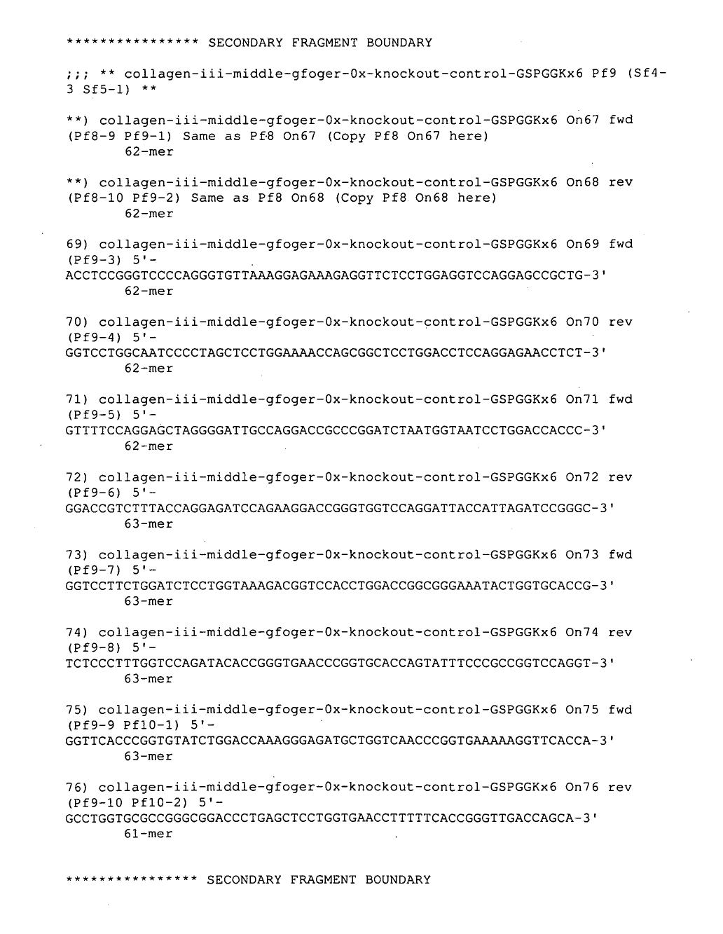

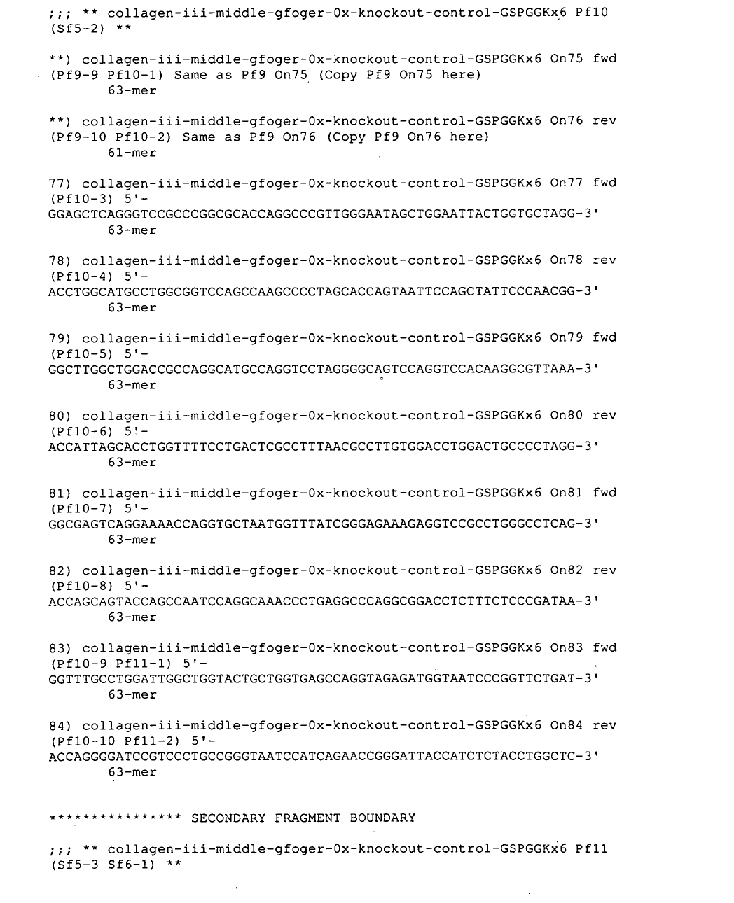

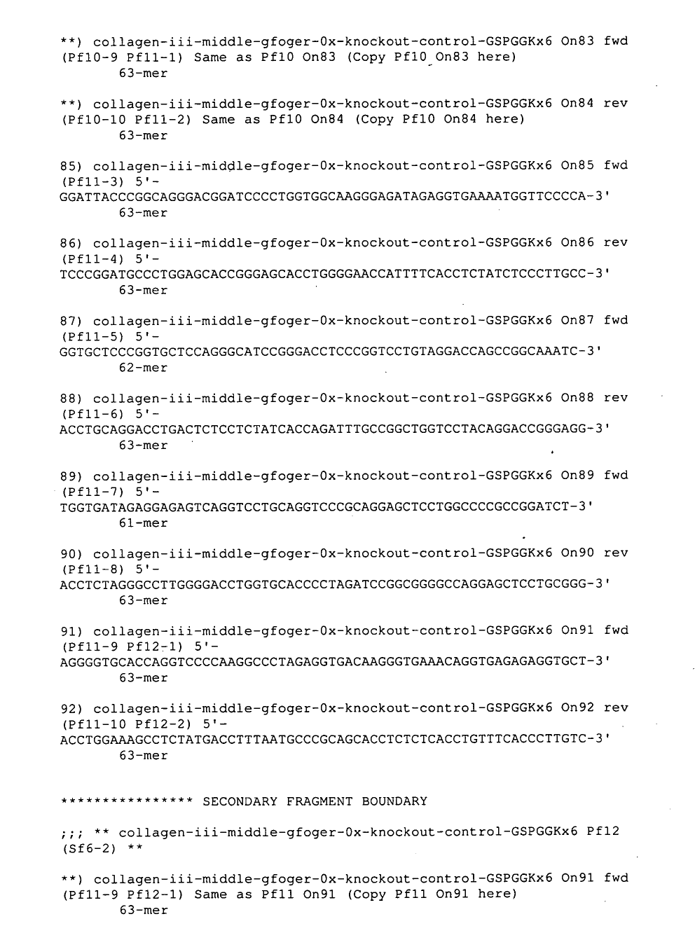

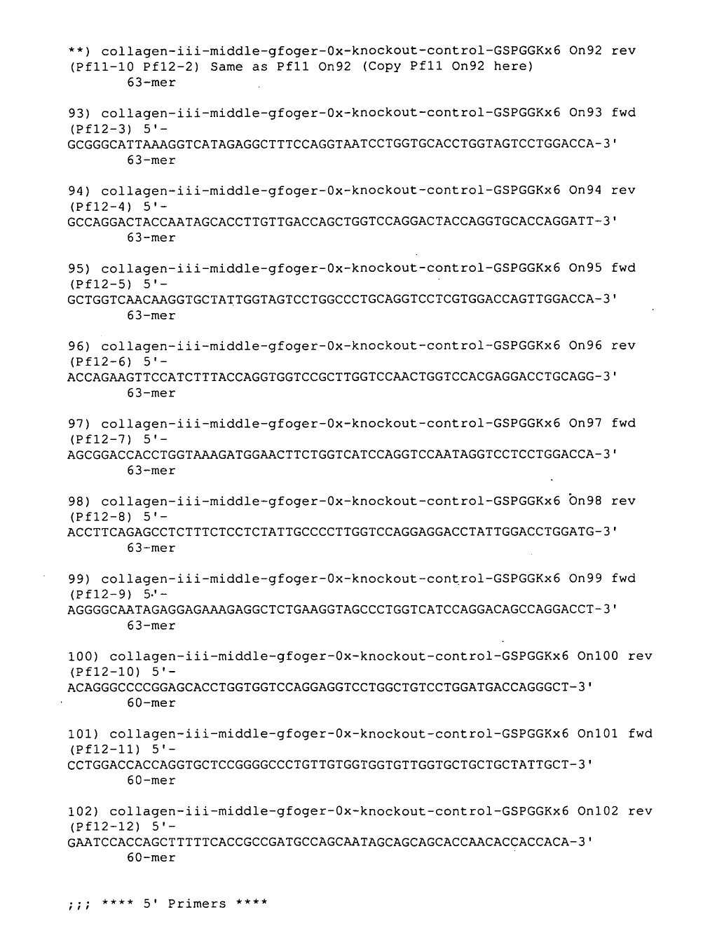

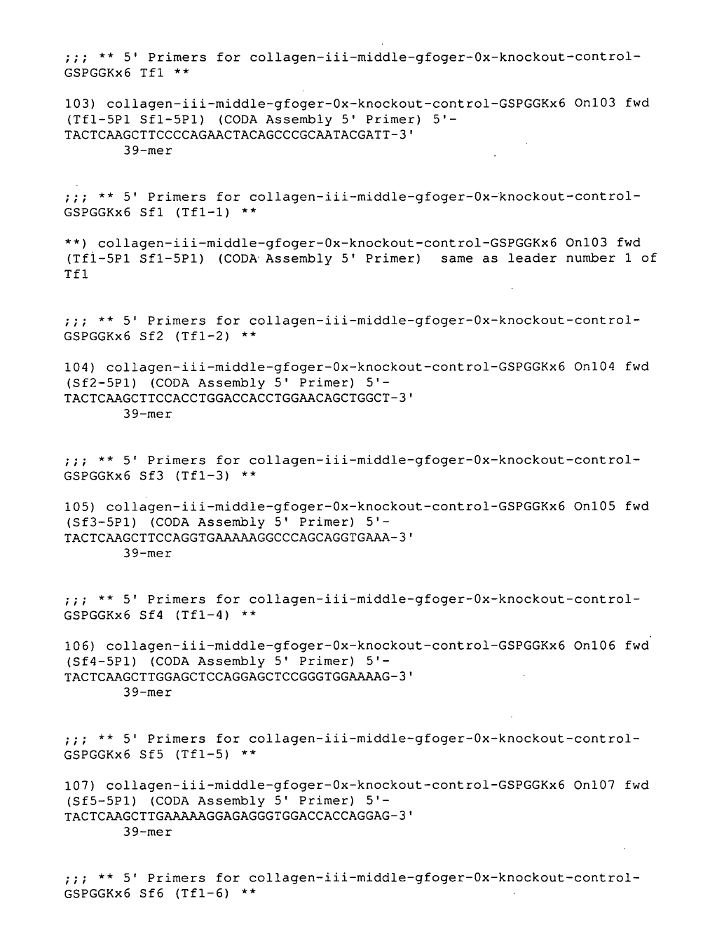

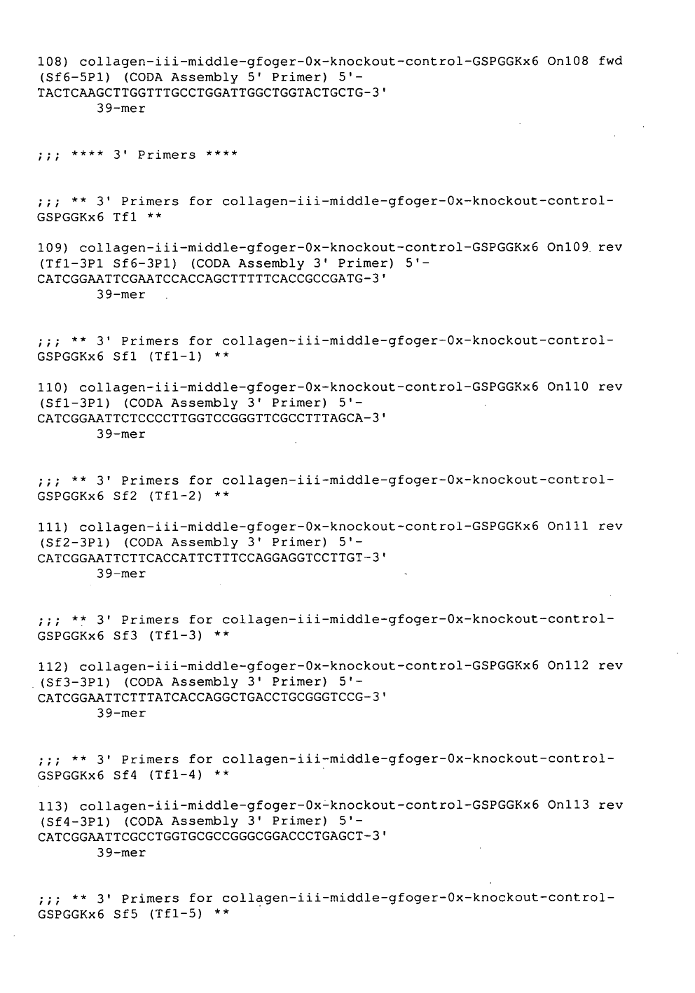

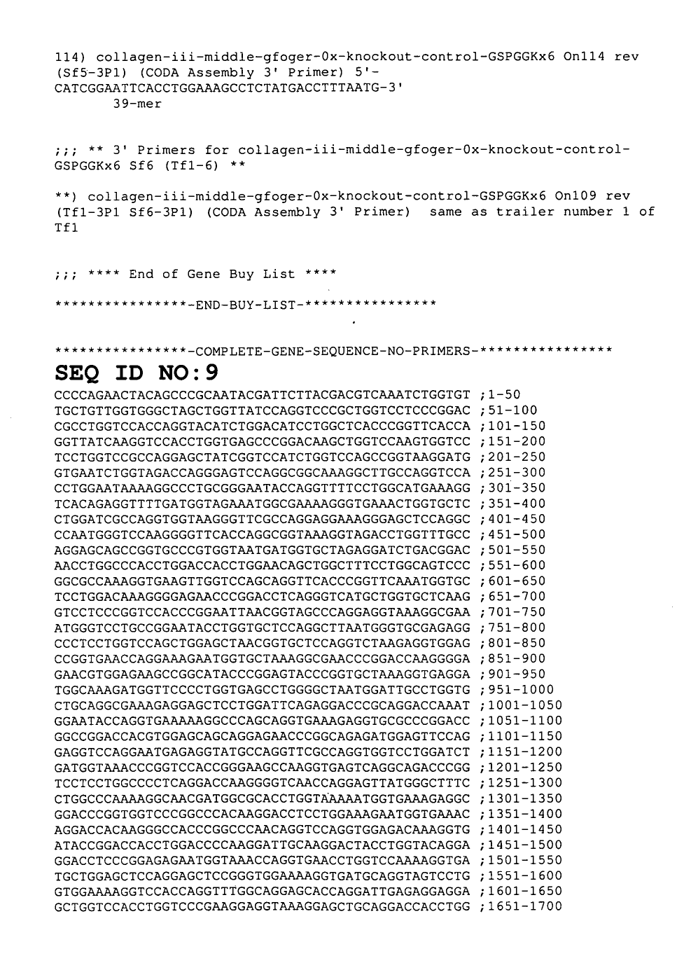

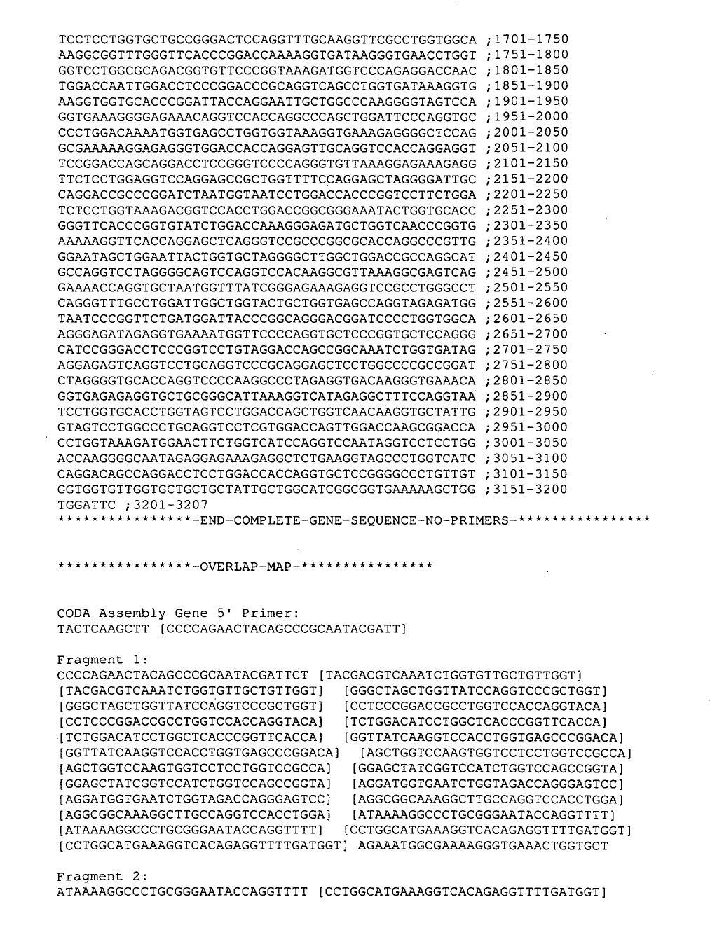

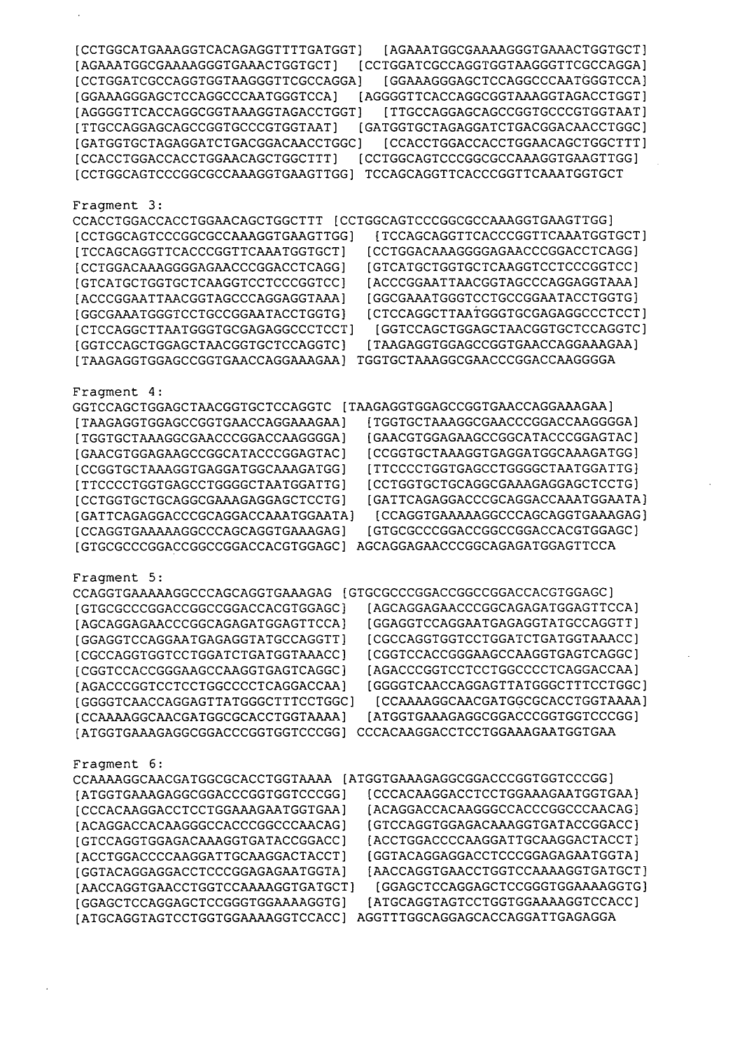

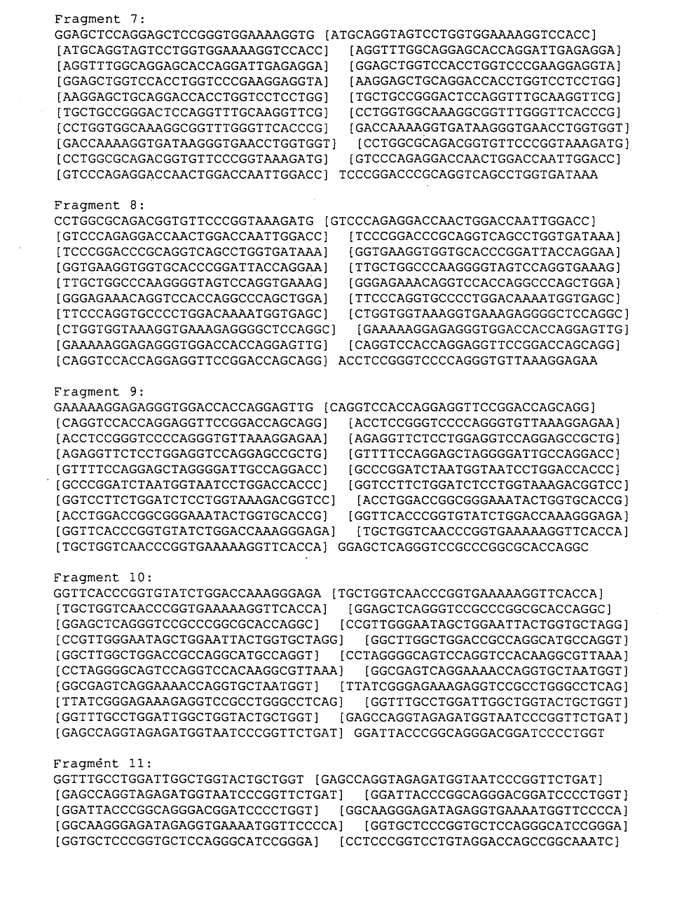

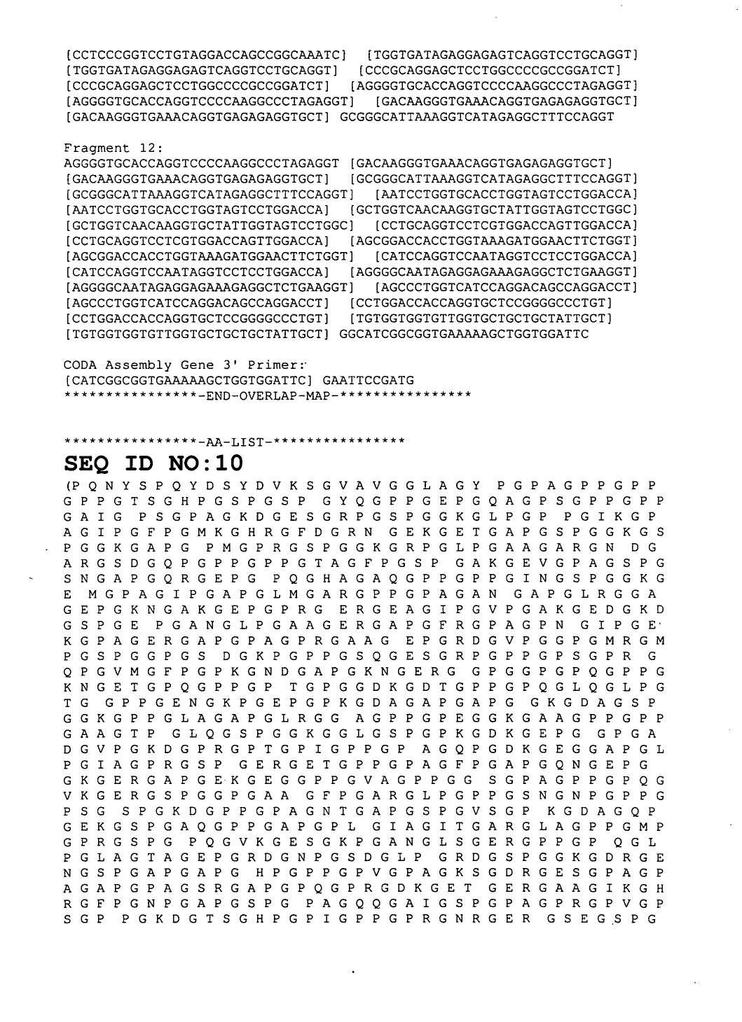

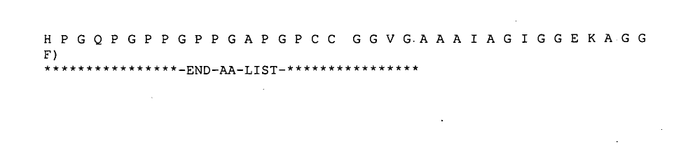

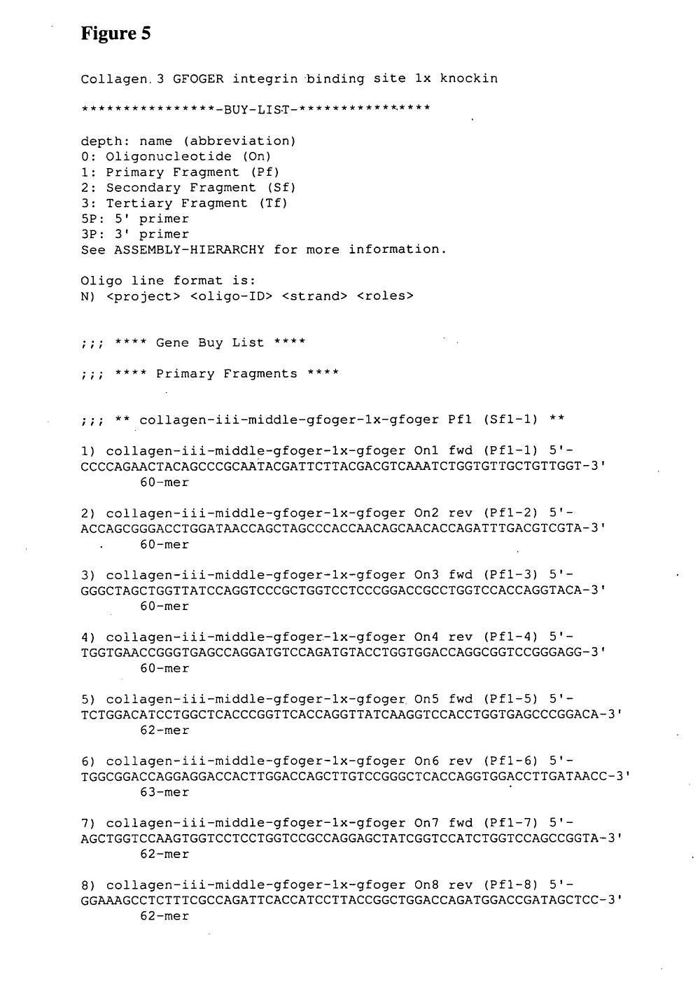

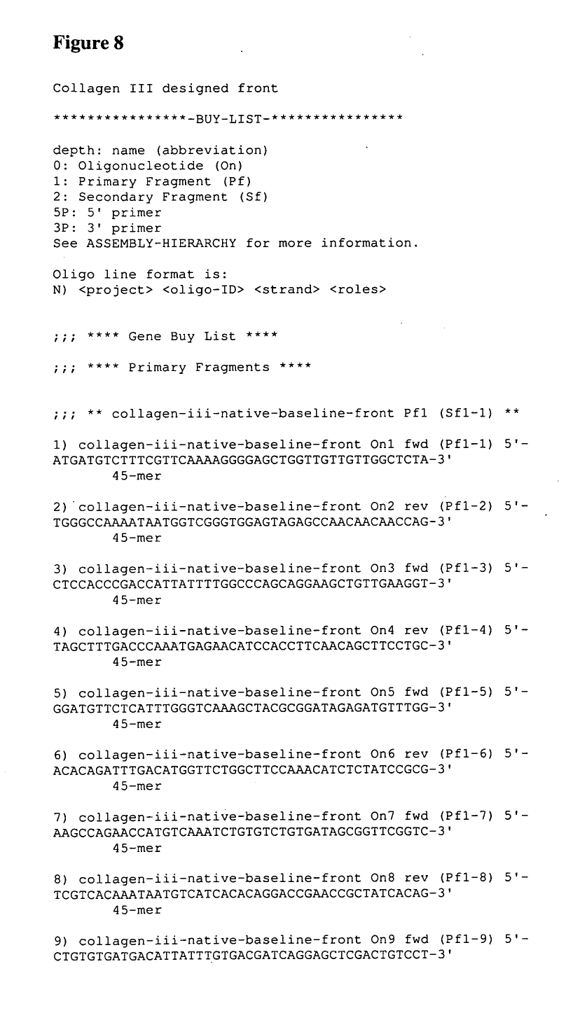

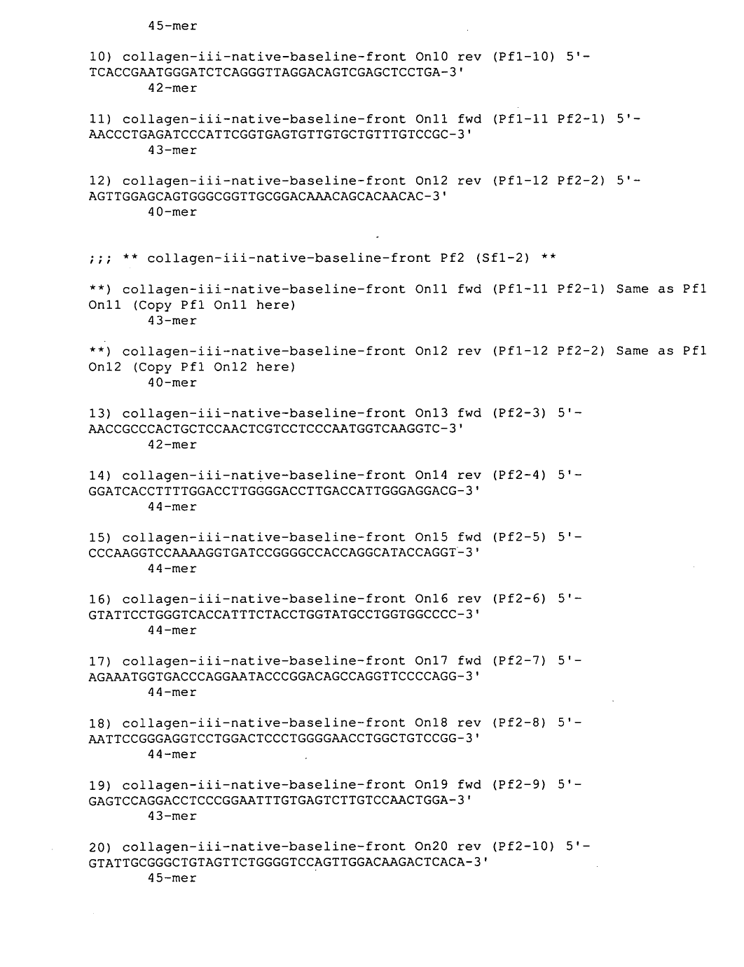

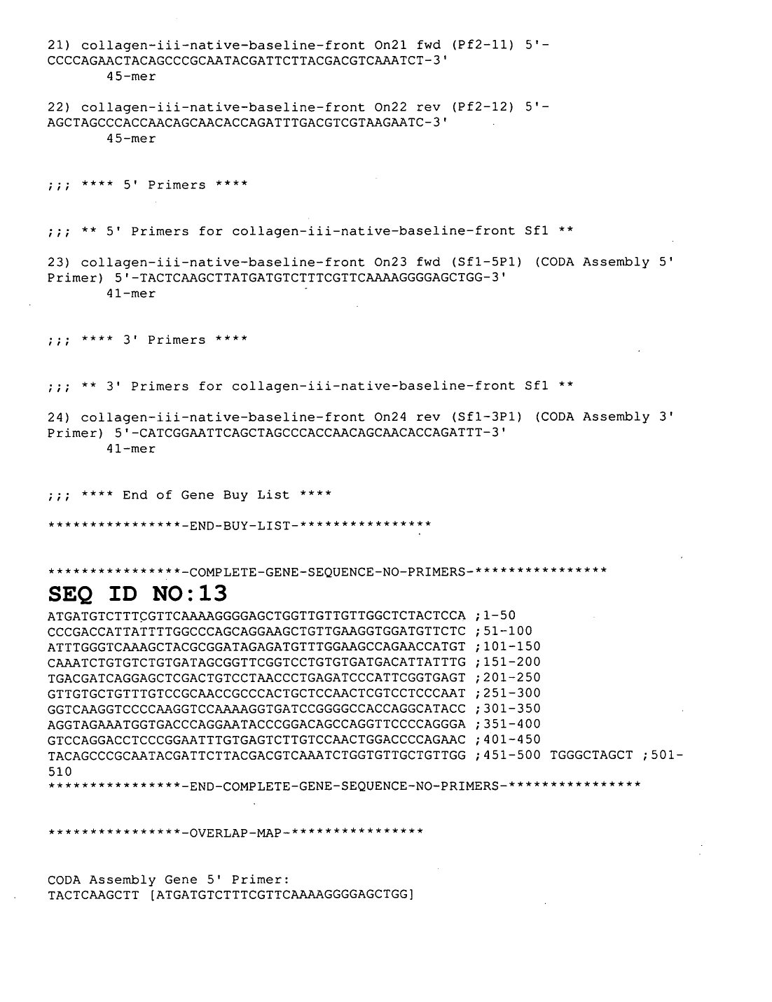

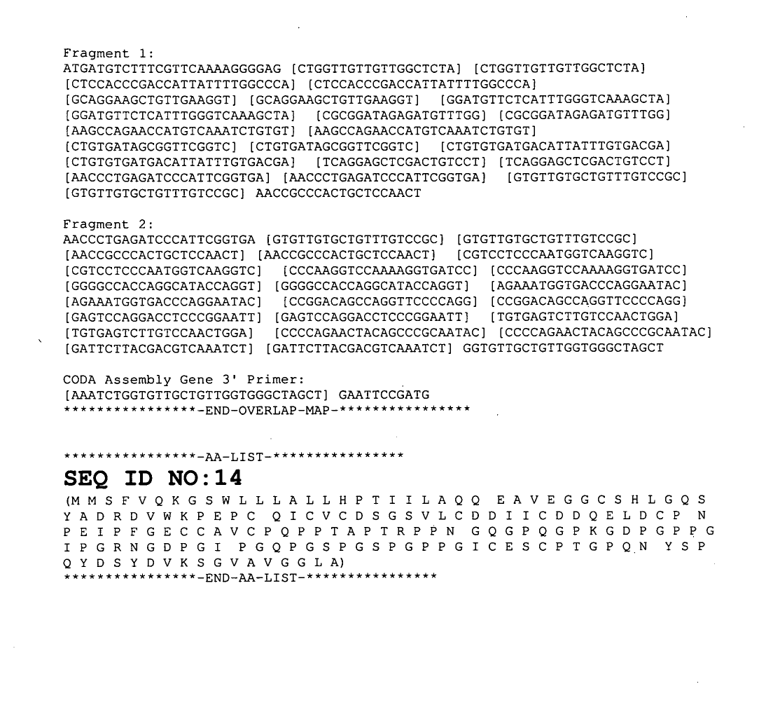

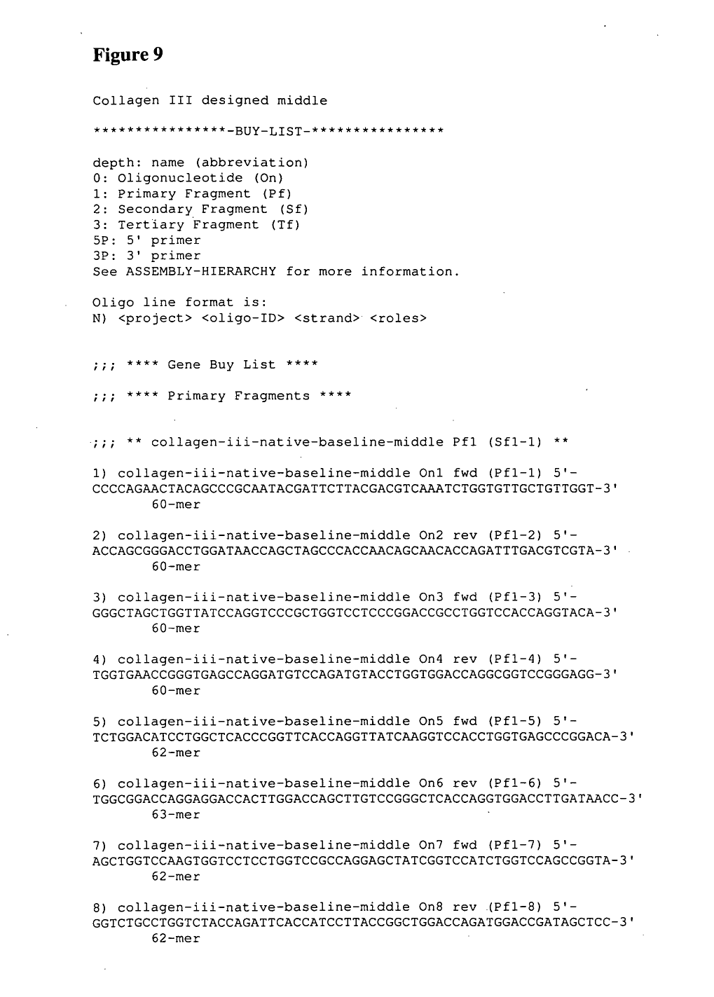

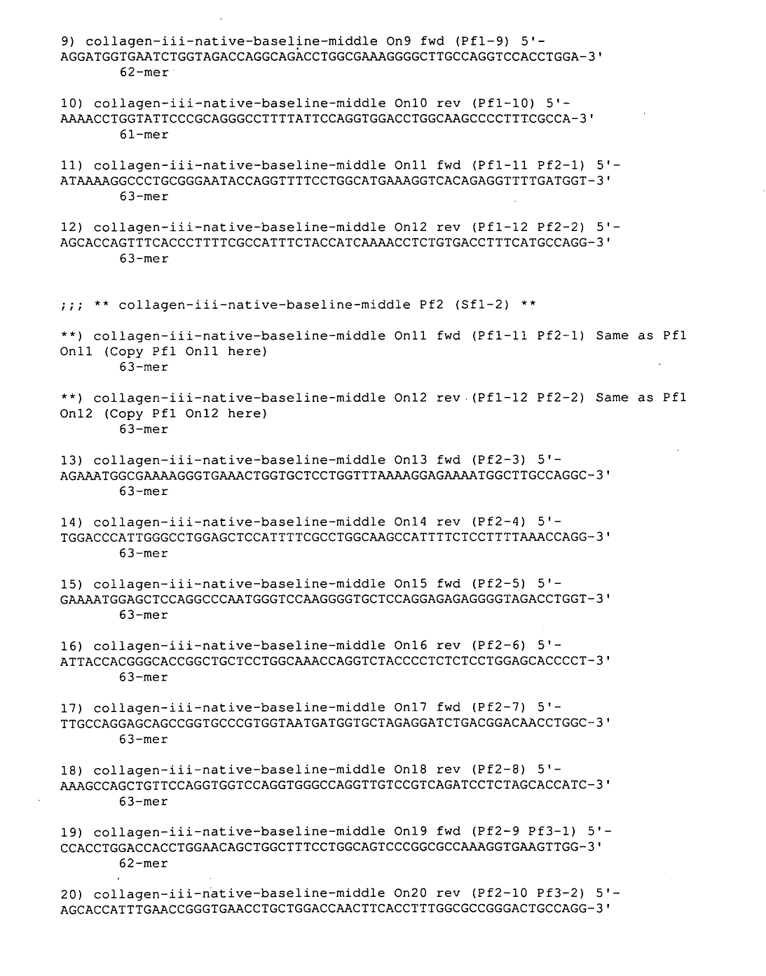

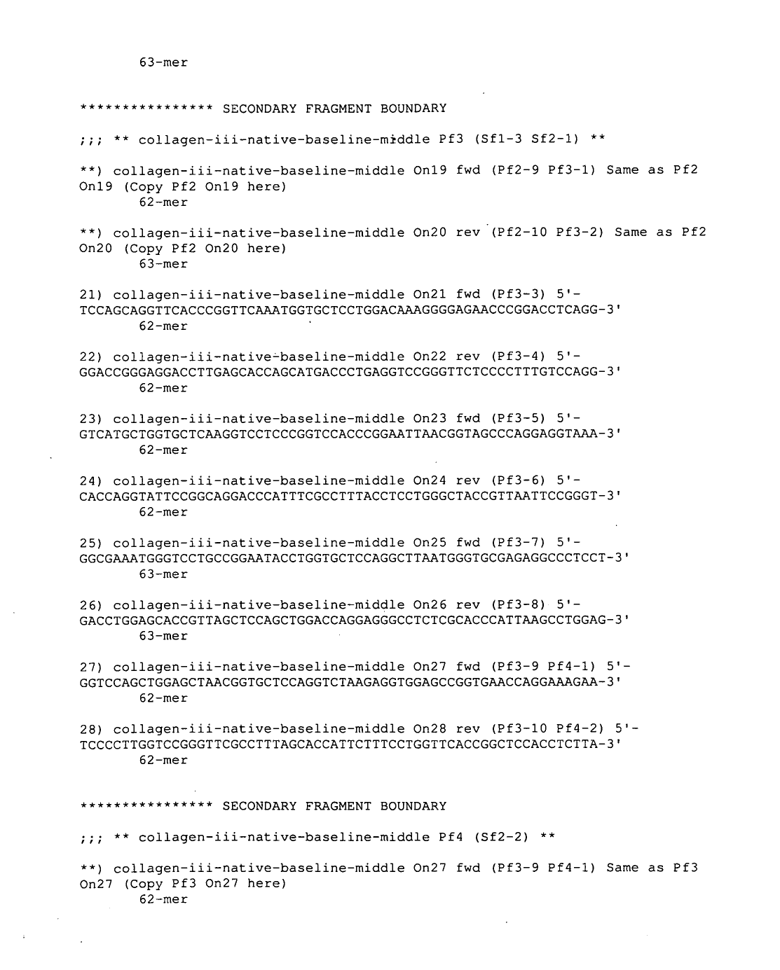

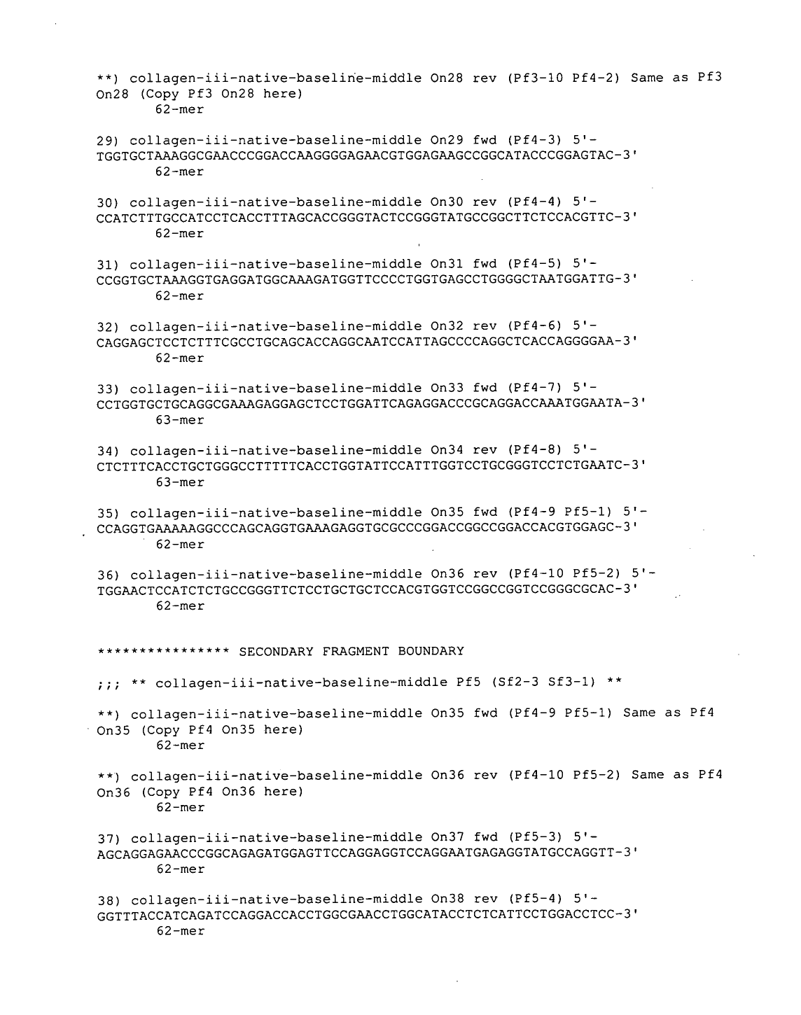

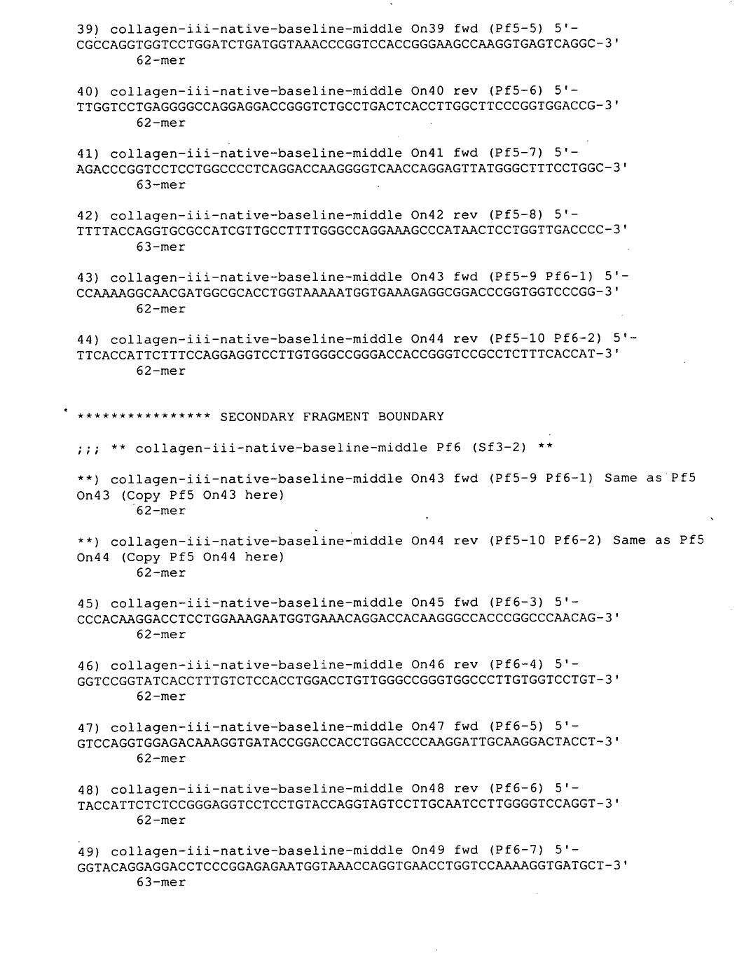

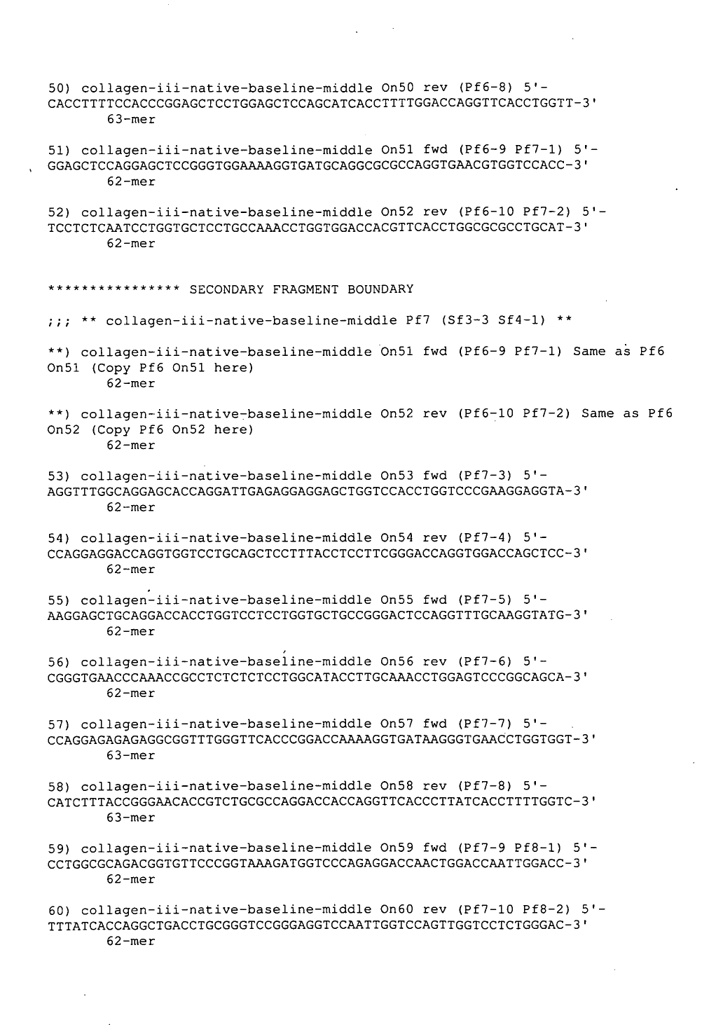

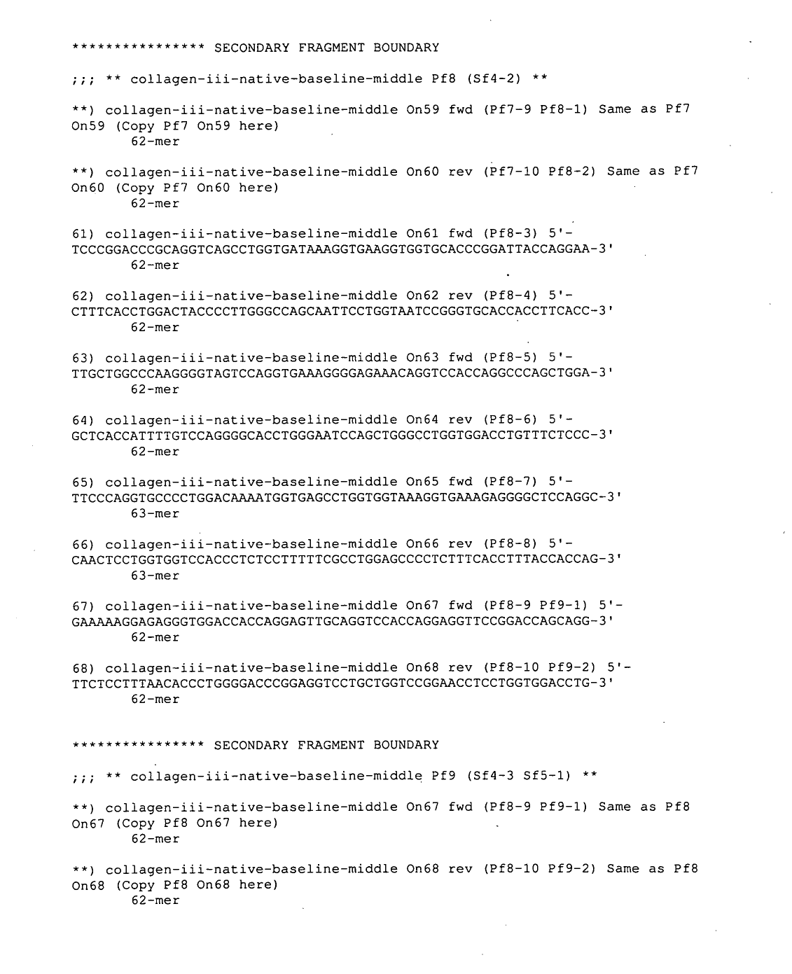

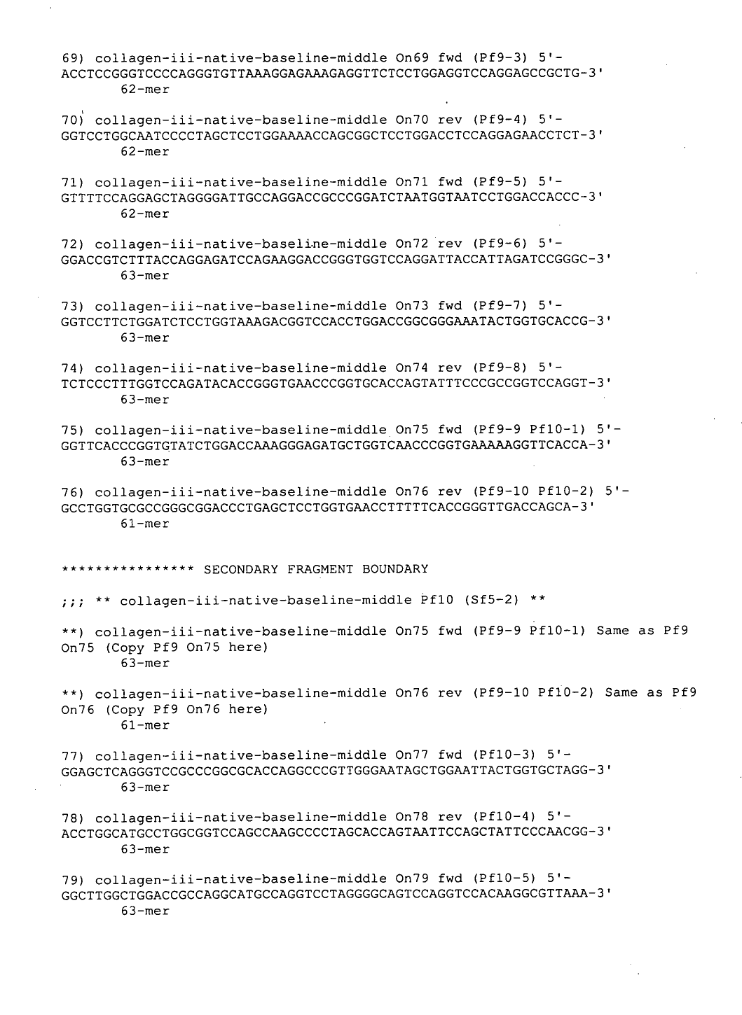

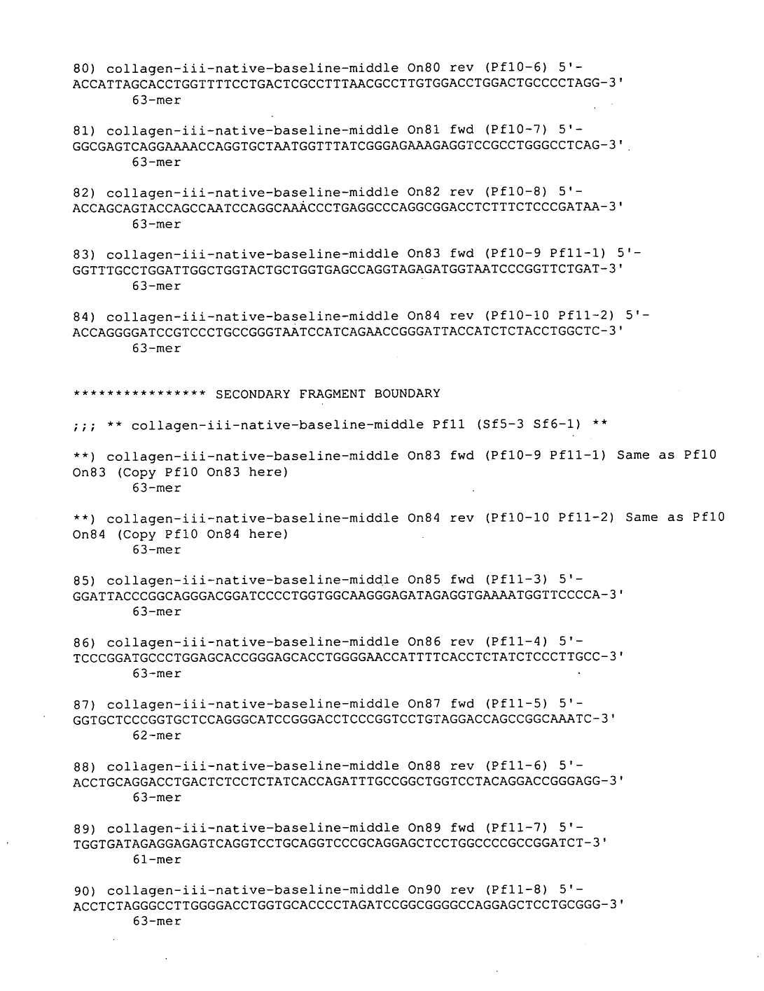

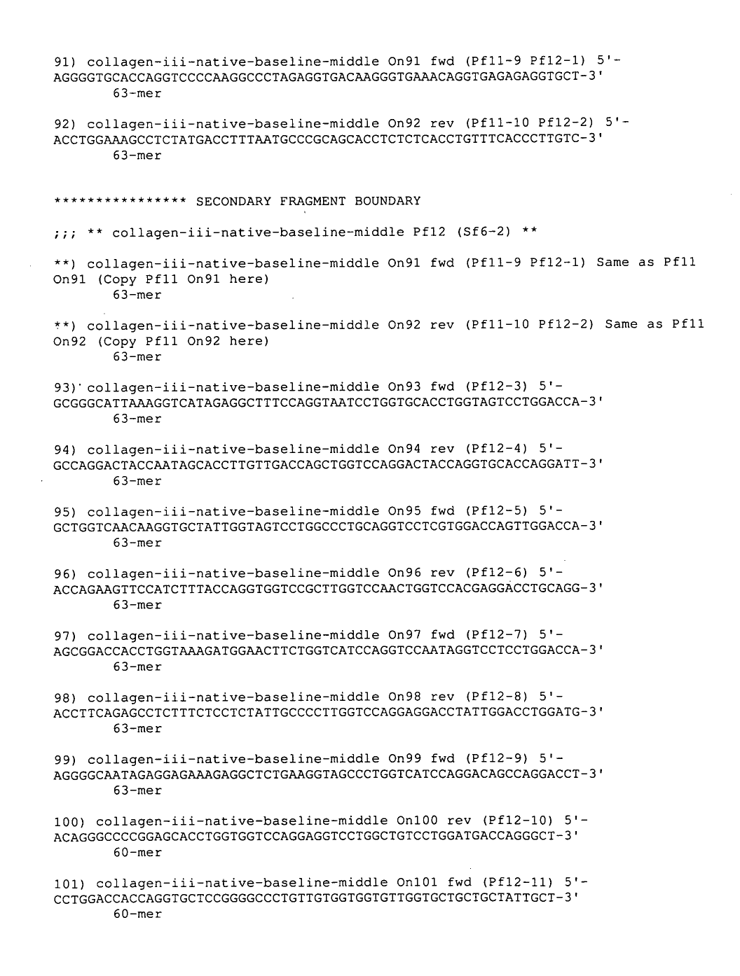

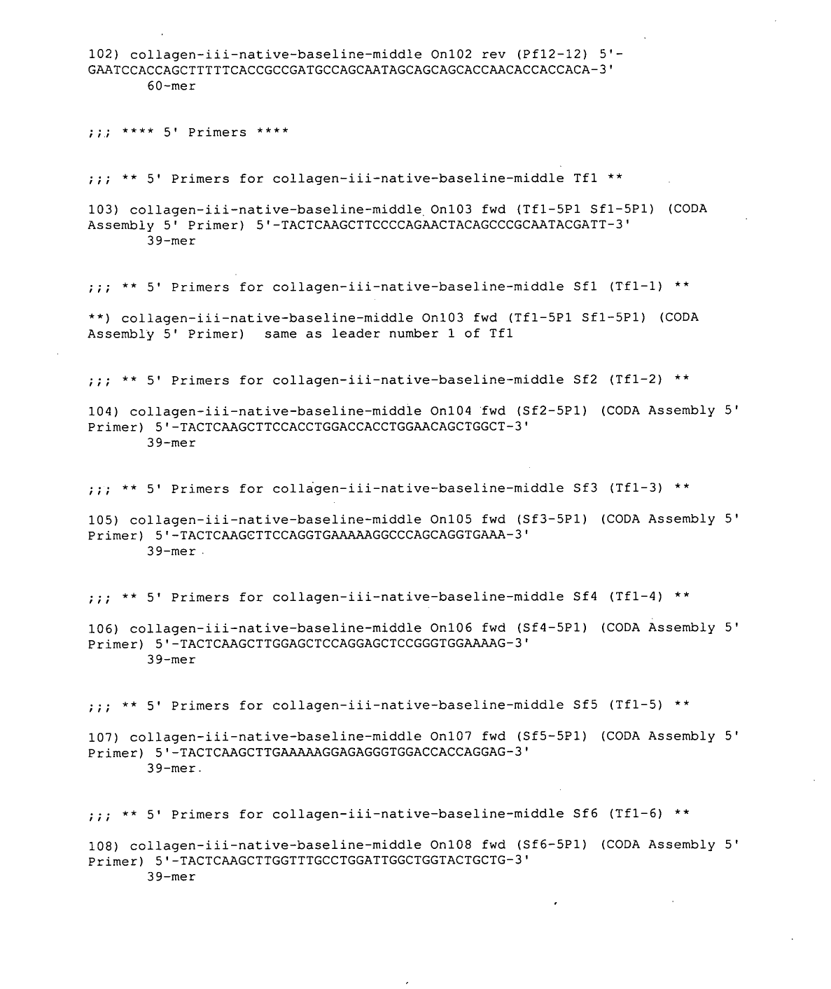

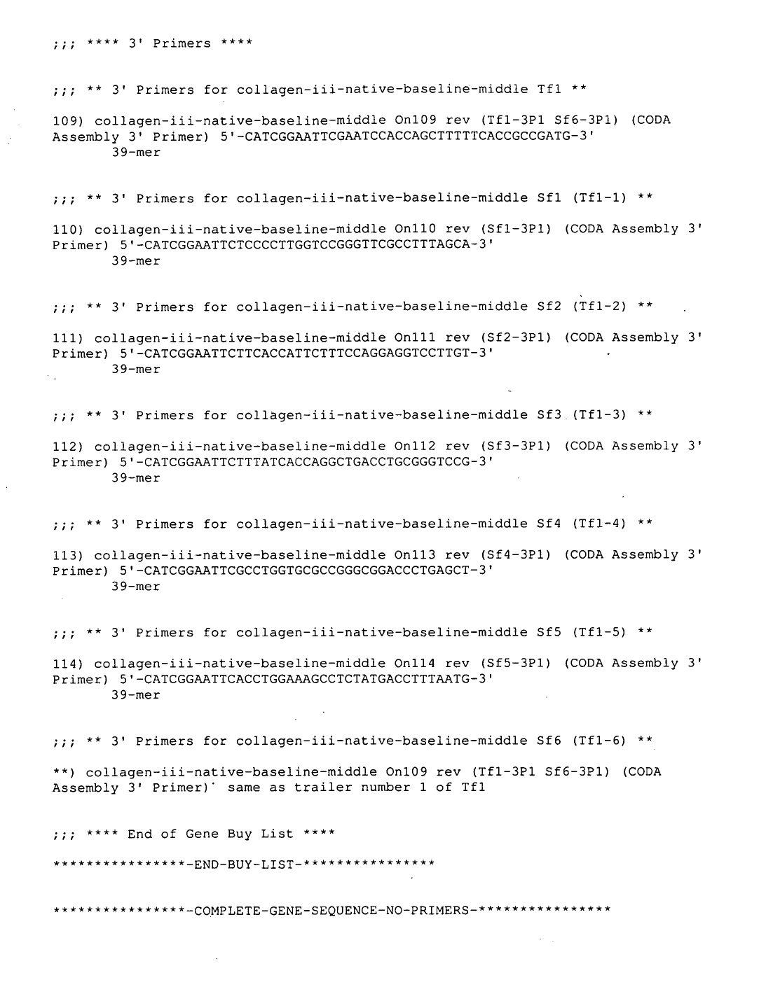

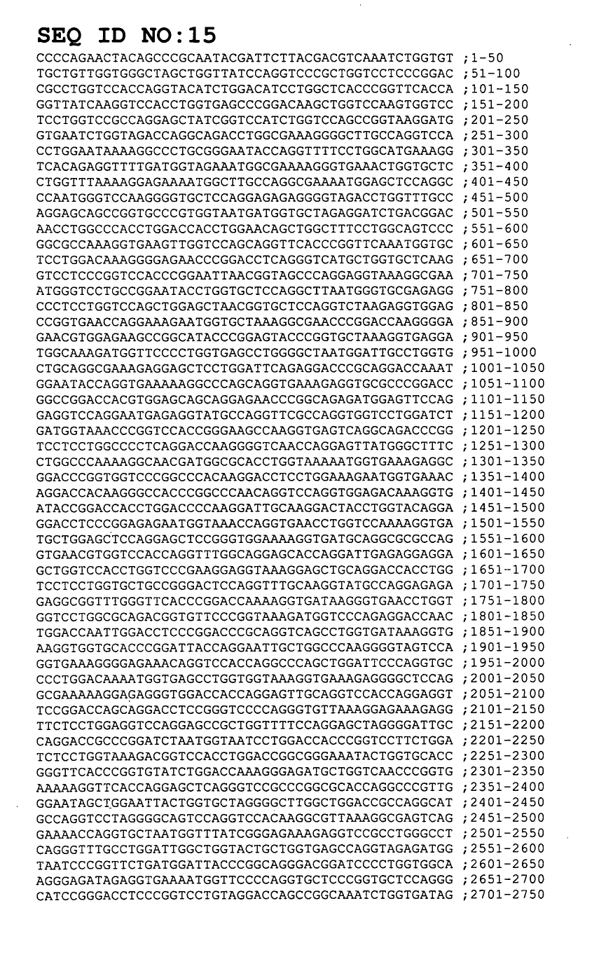

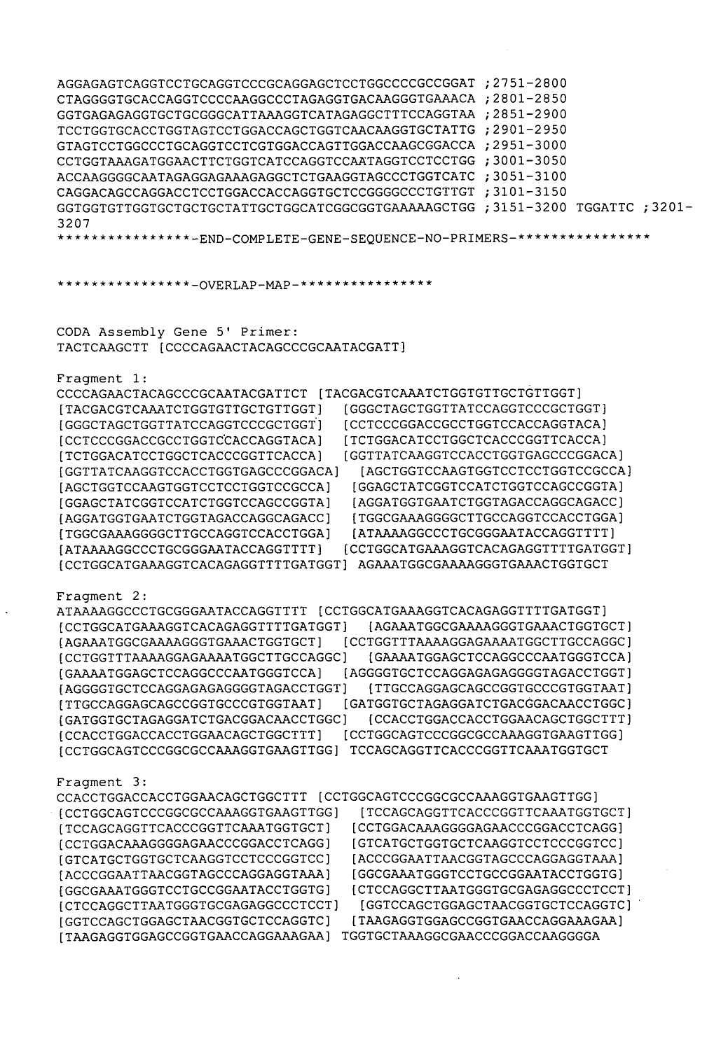

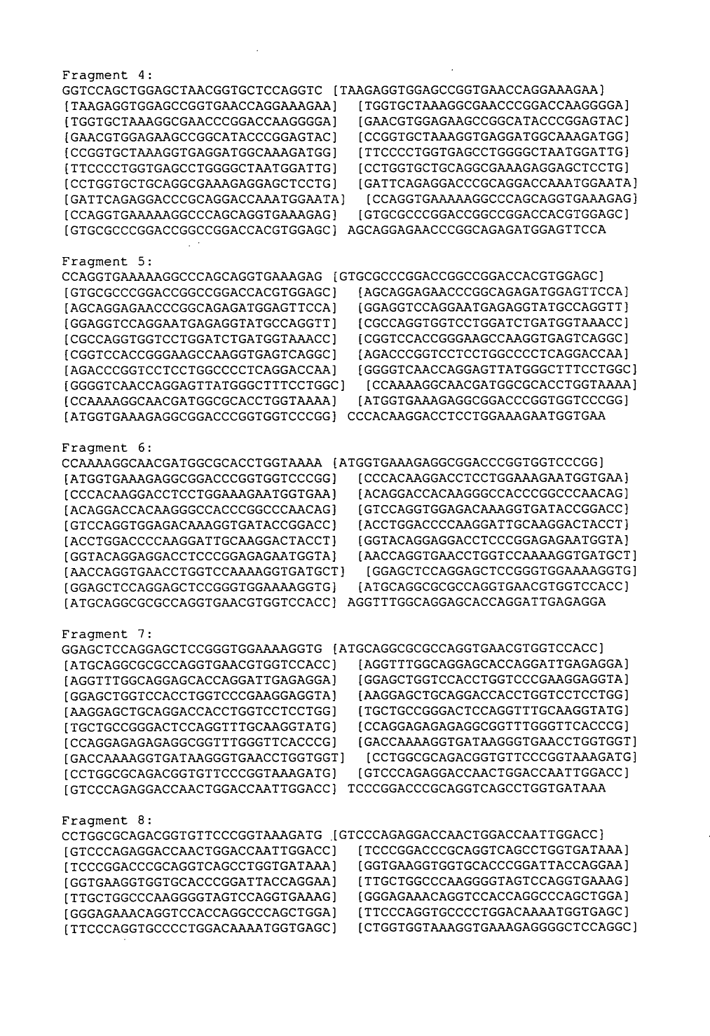

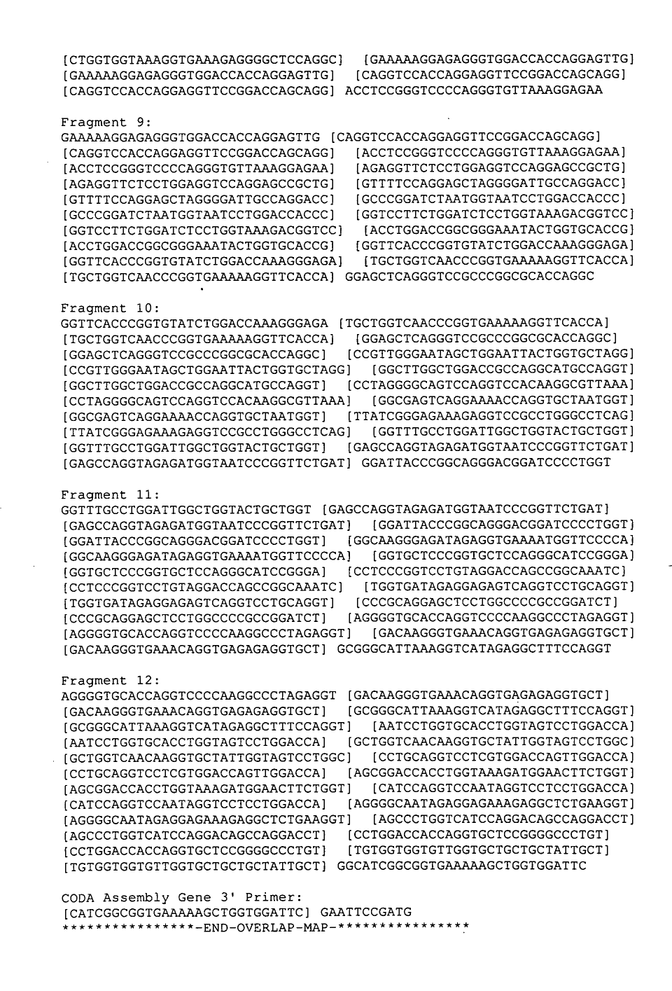

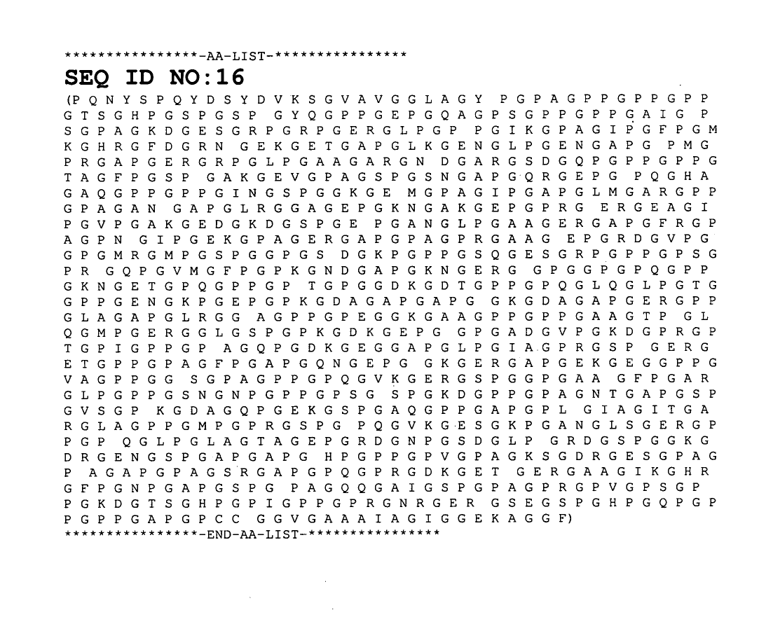

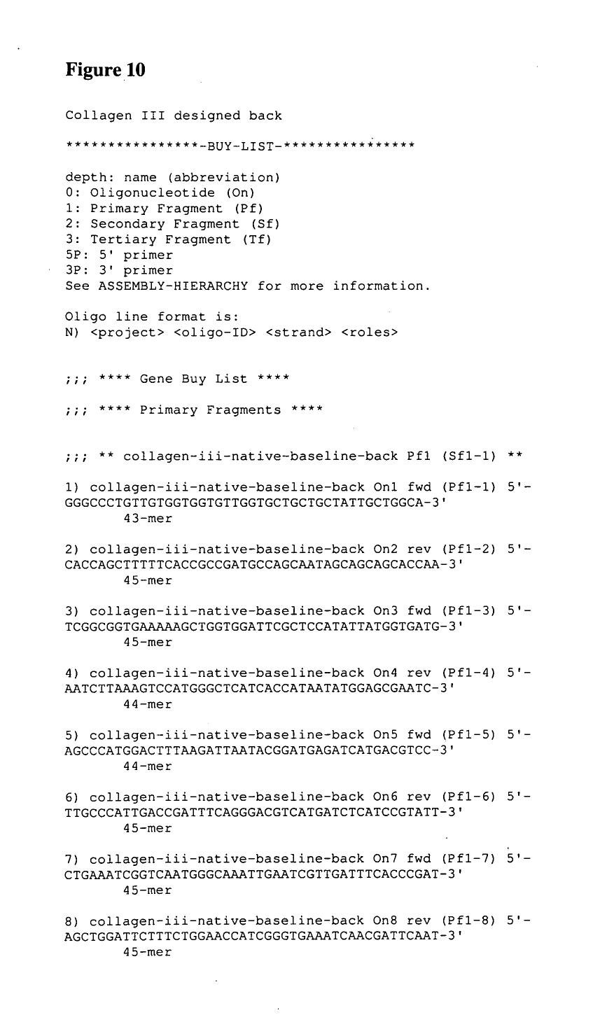

Examples of nucleotide sequences of polynucleotides provided herein are disclosed in SEQ ID NOs: 3, 5, 7, 9, and 11, which encode the amino acid sequences of SEQ ID NOs: 4, 6, 8, 10, and 12, respectively. These sequences all relate to known human collagen polypeptide sequences and the corresponding nucleotide sequences were designed according to the methods herein. SEQ ID NO: 3 discloses a nucleotide sequence encoding the amino acid sequence of the human collagen II alpha middle helical domain (SEQ ID NO:4). The oligonucleotide synthesis and assembly strategy for SEQ ID NO: 3 is provided in FIG. 1. SEQ ID NO: 5 discloses a nucleotide sequence encoding the amino acid sequence of the human collagen 1 alpha middle helical domain (SEQ ID NO:6). The oligonucleotide synthesis and assembly strategy for SEQ ID NO: 5 is provided in FIG. 2. SEQ ID NO: 7 discloses a nucleotide sequence encoding the amino acid sequence of the human collagen III alpha middle helical domain, with two amino acid substitutions to add cysteine residues at a285c and g581c (SEQ ID NO:6). The oligonucleotide synthesis and assembly strategy for SEQ ID NO: 7 is provided in FIG. 3. SEQ ID NO: 9 discloses a nucleotide sequence encoding the amino acid sequence of the human collagen III alpha middle helical domain, with putative integrin binding sites (GRPGER, GAPGER, GAPGER, GMPGER) replaced with the sequence GSPGGK, and with the sequence GLKGENGLPGEN replaced with the sequence GSPGGKGSPGGK (see SEQ ID NO:10), such that the designed sequence is proposed to contain no integrin binding sites. The oligonucleotide synthesis and assembly strategy for SEQ ID NO: 9 is provided in FIG. 4. SEQ ID NO: 11 discloses a nucleotide sequence encoding the amino acid sequence of the human collagen III alpha middle helical domain of SEQ ID NO:9, with a heterologous collagen I integrin binding site-containing sequence GERGFPGERGVQ inserted for the sequence GRPGRPGERGLP (see SEQ ID NO:12), such that the designed sequence is proposed to contain no endogenous integrin binding sites and one heterologous collagen I integrin binding site. The oligonucleotide synthesis and assembly strategy for SEQ ID NO: 11 is provided in FIG. 5. SEQ ID NO: 13 discloses a nucleotide sequence encoding the amino acid sequence of the N-terminal portion of human collagen III alpha, including up to the first three amino acids of the middle helical domain (SEQ ID NO:14). The oligonucleotide synthesis and assembly strategy for SEQ ID NO: 13 is provided in FIG. 8. SEQ ID NO: 15 discloses a nucleotide sequence encoding the amino acid sequence of the middle helical domain of human collagen III alpha, including the last twenty amino acids of the N-terminal portion and the first twenty amino acids of the C-terminal portion (SEQ ID NO:16). The oligonucleotide synthesis and assembly strategy for SEQ ID NO: 15 is provided in FIG. 9. SEQ ID NO: 17 discloses a nucleotide sequence encoding the amino acid sequence of the C-terminal portion of human collagen III alpha, including up to the last three amino acids of the middle helical domain (SEQ ID NO:18). The oligonucleotide synthesis and assembly strategy for SEQ ID NO: 17 is provided in FIG. 10.

In addition to the above, proteolytic sites (and, thus, nucleotides encoding such proteolytic sits) can be incorporated into the protein polymer. For incorporating proteolytic sites into the polymers, the ability of matrix metalloproteinases (MMP) to degrade collagen is utilized. This family of enzymes is important in normal processes such as tissue morphogenesis in embryonic development and in the repair of wounded tissues, and is overexpressed in tumor cells (or in their neighboring stromal cells). High levels of MMP have been correlated to metastasis behavior and the severity of the disease, and these studies have demonstrated that the degradation of extracellular matrix due to MMP leads to the invasion of malignant cells into other areas of the body. One of the most extensively-studied interstitial collagenases is MMP-1, which shows specificity towards cleavage of collagen types I, II, and III, with collagen III being the most rapidly degraded. One MMP-1 peptide cleavage sequence has been identified in human collagen III (Gly-Ala-Hyp-Gly-Pro-Leu-Gly-Ile-Ala-Gly-Ile-Thr-Gly-Ala-Ala) and the cleavage kinetics have been quantified.

It will readily be understood by those skilled in the art that various combinations of the polynucleotide sequence characteristics provided above are also contemplated herein, such that the polynucleotides provided herein possess at least two or more of the characteristics provided above, provided that the characteristics are not mutually exclusive.

Also provided herein are isolated polypeptides comprising an amino acid sequence having sequence similarity to a naturally occurring collagen polypeptide and comprising a plurality of amino acid changes relative to the naturally occurring collagen polypeptide. The sequence similarity can be at least, or at least about, 50%, 55%, 60%, 65%, 70%, 75%, 80%, 85%, 90%, 95%, 96%, 97%, 98%, 99%, 99.5%, 99.8% or 99.9% identical after alignment to an amino acid sequence of a naturally occurring collagen polypeptide. An amino acid change can include a site mutation, a deletion, and/or an insertion. Examples of particular amino acid changes are discussed elsewhere herein, and include, but are not limited to, a protease recognition site, an integrin binding site, a kinase target site, growth factor binding site, a cell binding site, and a chemical attachment site. In some embodiments, the polypeptide comprises more than 3, 4, 5, 6, 7, 8, 9, 10, 11, 12, 13, 14, 15, 16, 17, 18, 19, 20, 22, 24, 26, 28, 30, 35, 40, 45, 50, 55, 60, 70, 80, 90, or 100 amino acid changes relative to a naturally occurring collagen polypeptide with which it shares sequence similarity.

Also provided herein are isolated polypeptides comprising a collagen-like polypeptide domain, where collagen-like polypeptide domain contains Gly-X-Y triads, where “Gly” is the amino acid glycine, and X and Y symbolize individual amino acids. The description of the Gly-X-Y domains provided herein above in regard to the polynucleotide applies equally to the polypeptide and is expressly incorporated by reference.

In some polypeptides provided herein, the collagen-like polypeptide domain is at least, or at least about, 300, 350, 400, 450, 500, 550, 600, 650, 700, 750, 800, 850, 900, 950, 1100, 1200, 1300, 1400, or 1500 amino acids in length. Such a collagen-like polypeptide can be, for example, a collagen-like core helical domain.

Also provided herein are methods of designing collagen-like polypeptide encoding nucleotide sequences. The methods provided herein are based on the methods described in U.S. Pat. No. 7,262,031, and U.S. Patent Publication Numbers 2005/0106590 and 2007/0009928, which are hereby incorporated by reference in their entireties. The methods provided herein are modifications of, and improvements over, the above-referenced methods for purposes of design of collagen-like polypeptide encoding nucleotides. Collagen is an extremely difficult molecule to synthesize because of its repeating structure in the middle triple helical domain, at both the micro and macro scales. The micro scale amino acid sequence is a repeating sequence G-X-Y where G is glycine and X and Y are likely to be proline. Because glycine is encoded by nucleotides GGN and proline is encoded by nucleotides CCN, the GG/CC pairs are prone to form incorrect hybridizations which lead to deleterious incorrect polymerase extension and consequently incorrect product. The macro scale amino acid sequence contains many direct sequence repeats, of which for human collagen-III the largest is GQPGPPGPPG occurring at nucleotide locations 163 and 1015 (or, equivalently, at amino acid locations 55 and 339) within the middle triple helical domain. Numerous other direct sequence repeats in human collagen-III also are present.

Several method improvements beyond those disclosed in U.S. Pat. No. 7,262,031 (Lathrop & Hatfield) were implemented to address such extensive direct sequence repeats. First, a method for efficiently finding sequence repeats was used to identify potential sequence repeat problems in advance. This method is described in Karlin S, Ghandour G, Ost F, Tavare S, Korn L J, “New approaches for computer analysis of nucleic acid sequences,” Proc. Natl. Acad. Sci. USA, 1983 September; 80(18):5660-4, which is incorporated by reference in its entirety. Second, a stochastic opportunistic local search method, hill climbing with random restarts, was used to supplement the best-first search method disclosed in U.S. Pat. No. 7,262,031 (Lathrop & Hatfield). Hill climbing with random restarts is known in the art, for example, Artificial Intelligence, 3rd edition, by Patrick Henry Winston, 1992 (from the Addison-Wesley series in computer science). Third, a pattern recognition method was used to encode and recognize in the sequence the incorrect hybridizations referred to above. Pattern recognition methods are known in the art, for example, Artificial Intelligence, 3rd edition, op. cit.

The repeat-finding method of Karlin, et al., (2008) identifies amino acid direct repeats during a preprocessing step. Each amino acid direct repeat corresponds to a large number of nucleotide direct repeats, one possible nucleotide repeat for each possible assignment of codons to amino acids in the repeat such that corresponding amino acids are assigned identical codons. Conversely, the possible nucleotide repeats can be disrupted by assigning different codons to corresponding amino acids in the repeat. The preprocessing initializes each amino acid in each repeat to a default candidate codon that is chosen in order to minimize the length and number of amino acids in the repeat that are assigned identical codons. Multiple repeats are considered simultaneously. When it is possible to assign a default candidate codon to an amino acid in the repeat without increasing the length and number of identically assigned codons, the most prevalent codon (highest genomic codon usage) is assigned. When it is not possible, a default candidate codon is chosen and assigned arbitrarily by a random process that is somewhat biased to prefer more prevalent codons. The result is that preprocessing assigns all amino acids in direct repeats a default candidate codon that minimizes the number and length of nucleotide direct repeats, while simultaneously somewhat biasing the codon assignments to favor more prevalent codons that result in greater use of the most favored codons for the host organism.

The preprocessing step also stores in memory a machine representation of the amino acid direct repeats. This representation is used later, during the sequence optimization search using the methods described in U.S. Pat. No. 7,262,031, and U.S. Patent Publication Numbers 2005/0106590 and 2007/0009928, to improve the efficiency with which the sequence optimization search recognizes and avoids nucleotide direct sequence repeats. During the sequence optimization search, the initial default candidate codons are replaced by many different combinations of other candidate codons as the search proceeds toward a final solution of codon assignments that optimizes several desirable characteristics simultaneously, including correct thermodynamic self-assembly as described in U.S. Pat. No. 7,262,031, and U.S. Patent Publication Numbers 2005/0106590 and 2007/0009928. After each iteration of a search step in which candidate codons are changed, the memory representation of amino acid direct repeats is consulted to determine which, if any, of the amino acid direct repeats contains a changed candidate codon. Each such amino acid repeat with changed codons is checked to determine whether its nucleotide sequence has been changed to include a deleterious nucleotide direct repeat, and if so the codon changes involved are rejected. The result is that changed combinations of candidate codons are accepted only if they do not lead to a deleterious nucleotide direct repeat. Consequently, the sequence optimization search avoids all deleterious nucleotide direct repeats while optimizing several desirable characteristics simultaneously, and does so rapidly and efficiently.

Search speed and efficiency is greatly increased by a stochastic opportunistic local search method, hill climbing with random restarts, which is now called at each iterative step of the best-first search method disclosed in U.S. Pat. No. 7,262,031 (Lathrop & Hatfield). Prior to each such step, a step preprocessing method constructs the current candidate codon assignment by assigning to each amino acid its currently fixed codon if its codon is currently fixed by the search, and otherwise assigning its initial default candidate codon. This current candidate codon assignment is examined to determine whether or not it satisfies all currently known optimization constraints, including those thermodynamic constraints already determined from previous sequence analysis as described in U.S. Pat. No. 7,262,031 (Lathrop & Hatfield). If so, then the preprocessing step terminates, the current candidate codon assignment is subjected to sequence analysis including a full thermodynamic analysis (which usually reveals new thermodynamic constraints not previously known), and the sequence optimization search described in U.S. Pat. No. 7,262,031 continues as before. If not, then the preprocessing step does not terminate, one of the codon positions in the current candidate codon assignment that was responsible for the failure is selected randomly, a new candidate codon is selected for that position by a random process that is somewhat biased to prefer more prevalent codons, and the process described above is repeated. In the preferred embodiment, the preprocessing step makes 50 such codon replacements for each current candidate codon assignment considered, and considers 20 current candidate codon assignments for each preprocessing step. In other embodiments, the preprocessing step makes 1, 2, 3, 4, 5, 7, 10, 15, 20, 25, 30, 40, 50, 75, 100, 150, 200, 300, 500, 1000, 5000, or 10000 such codon replacements for each current candidate codon assignment considered, and considers 1, 2, 3, 4, 5, 7, 10, 15, 20, 25, 30, 40, 50, 75, 100, 150, 200, 300, 500, 1000, 5000, or 10000 such current candidate codon assignments for each preprocessing step. The entire best-first search method repeats at each advance in the temperature gap as the sequence becomes optimized. The result is that the best-first search method disclosed in U.S. Pat. No. 7,262,031 (Lathrop & Hatfield) is augmented by a stochastic search method that provides numerous opportunities to discover a new satisfactory codon assignment very early in the search at each advance in the temperature gap, and often does so, thus greatly speeding the overall search for a final satisfactory codon assignment.

Patterns are used to encode efficiently all infeasible sequence constraints, all thermodynamic melting temperatures resulting from both correct and incorrect hybridizations, all instantiated nucleotide direct sequence repeats, and all many other constraints and conditions that occur in the search. A pattern consists of a list of codon positions named by the pattern, and for each codon position, a list of codons that may instantiate the pattern at that codon position. A pattern matches a sequence when each codon position named by the pattern is filled in the sequence by a codon appearing in the list of codons that may instantiate that codon position. When a pattern corresponding to an infeasible sequence constraint matches a sequence, that sequence is declared infeasible and is pruned. When a pattern corresponding to a correct or incorrect hybridization matches a sequence, that sequence is declared to have achieved the corresponding temperature either as a correct or an incorrect hybridization. When any other pattern type matches a sequence, the sequence is processed according to the action specified by that pattern type. The result is that a great deal of previously accumulated knowledge about the search space and its constraints can be encoded efficiently and applied rapidly during the search. For example, patterns are used to guide the stochastic and the best first search methods mentioned above.

Pattern-based constraint discovery is used when a new infeasible thermodynamic constraint is encountered and codified as a pattern. For each encoded pattern, four exploratory optimizations are used to explore alternative hybridization patterns and thereby improve the search method. First, all possible amino acid silent codon substitution combinations are made at the endpoints of each helix encoded by that pattern. A substitution at a helix endpoint corresponds to an alternative helix terminal mismatched pair as described in Algorithms and Thermodynamics for RNA Secondary Structure Prediction: A Practical Guide, by M. Zuker, D. H. Matthews, and D. H. Turner (which serves as a manual for the software package mfold developed by M. Zuker). Second, all possible amino acid silent codon substitutions are made at each internal amino acid position individually. A substitution at an internal amino acid position corresponds to a different pattern of nucleotides at that codon position. Third, each amino acid codon position is dropped from the pattern in turn. This corresponds to identifying and omitting codon positions that do not contribute to the incorrect hybridization, and therefore can be ignored by the stochastic opportunistic local search method described above. Fourth, all possible internal amino acid silent codon substitution combinations are made that maintain the original pattern hybridizations. This corresponds to identifying all other equivalent patterns at that location.

Based on these results, oligonucleotide sequence design is performed according to the methods described in U.S. Pat. No. 7,262,031, and U.S. Patent Publication Numbers 2005/0106590 and 2007/0009928, which are hereby incorporated by reference in their entireties.

### Collagen

The term collagen refers to any one of the known collagen types, including collagen types I through XXIII, as well as to any other collagens, whether natural, synthetic, semi-synthetic, or recombinant. The term also encompasses procollagens and preprocollagens (e.g., procollagens with signal peptide). The term collagen encompasses any single-chain polypeptide encoded by a single polynucleotide, as well as homotrimeric and heterotrimeric assemblies of collagen chains. The term “collagen” specifically encompasses variants and fragments thereof, and functional equivalents and derivatives thereof, which preferably retain at least one structural or functional characteristic of collagen, for example, a (Gly-X-Y)n.

The chain property of collagen responsible for this phenomenon is its ability to spontaneously form interchain aggregates having a conformation designated as a triple helix. The helices are stabilized by weak interactions between chains, arising from the close proximity of the peptide backbone at locations every third residue occupied by glycine and kinks provided by proline and hydroxyproline at the two positions between glycine. The geometry of the three kinked chains allows for hydrogen bonding within the triple helix. The structure is loose and is readily accessible to interaction with water, small organic and inorganic molecules, other proteins, and cells. Although collagen consists of many different amino acid sequences, one of the more structurally stable segments exists at the amino and carboxyl terminal ends of the processed collagen Type I chains. These ends consist to a large degree of the repeating tripeptide sequence GPP (the second P is often hydroxylated).

By contrast with natural forms of collagen, recombinantly-produced collagen-like polymers may consist exclusively of a single repeating tripeptide sequence selected from a wide variety of Gly-X-Y sequences, where X and Y can be any amino acid, whether derived from known natural sequences or not. Collagen-like polymers can also consist of different tripeptide sequences, which are repeated as blocks in the final polymer. Dissimilar blocks can also be used in a repeating fashion to create block copolymers in order to provide additional chemical or biological functionality.

The term procollagen refers to a procollagen corresponding to any one of the collagen types, whether natural, synthetic, semi-synthetic, or recombinant, that possesses additional C-terminal and/or N-terminal propeptides or telopeptides that assist in trimer assembly, solubility, purification, or any other function, and that then are subsequently cleaved by N-proteinase, C-proteinase, or other enzymes, e.g., proteolytic enzymes associated with collagen production. The term procollagen specifically encompasses variants and fragments thereof, and functional equivalents and derivatives thereof, which preferably retain at least one structural or functional characteristic of collagen, for example, a (Gly-X-Y), domain. Thus, a procollagen may comprise a core helical domain flanked by N- and/or C-terminal non helical “pro” domains.

Mature collagen is formed by the association of three procollagen monomers which include “pro” domains at the amino and carboxy terminal ends of the polypeptides. The pro domains are cleaved from the assembled procollagen trimer to create mature, or “telopeptide” collagen. The telopeptide domains may be removed by chemical or enzymatic means to create “atelopeptide” collagen.

Naturally occurring collagen refers to any collagen that occurs in nature. Thus, naturally occurring collagen includes the wild-type collagen sequence of any organism in nature. Such naturally occurring collagen would include any collagen of known amino acid sequence from an organism in nature. For example, collagens I-XXIII are known in human, fish and birds. Collagens I, II, III, IV, V and XI, for example, have been described and are known in the art. Among the known sequences of naturally occurring collagens is the nucleotide sequence of human collagen III (SEQ ID NO: 1), which encodes the human collagen III amino acid sequence (SEQ ID NO: 2). The nucleotide and corresponding amino acid sequences for a large number of naturally occurring collagens are known in the art.

### Collagen Types

Many distinct collagen types have been identified in vertebrates. These collagen types are numbered by Roman numerals and the chains found in each collagen type are identified with Arabic numerals. Over 90% of the collagen in humans, however, are Collagens I, II, III, and IV. A detailed description of structure and biological functions of the various different types of naturally occurring collagens can be found, among other places, in Ayad et al., The Extracellular Matrix Facts Book, Academic Press, San Diego, Calif., (1994); Burgeson, R. E., and Nimmi, “Collagen types: Molecular Structure and Tissue Distribution,” Clin. Orthop. 282: 250-272 (1992); Kielty, C. M. et al., “The Collagen Family: Structure, Assembly And Organization In The Extracellular Matrix,” in Connective Tissue And Its Heritable Disorders, Molecular Genetics, And Medical Aspects, Royce, P. M. and Steinmann, B., Eds., Wiley-Liss, NY, pp. 103-147 (1993).

Type I collagen is the major fibrillar collagen of bone and skin. Type I collagen is a heterotrimeric molecule comprising two α1(I) chains and one α2(I) chain. Details on preparing purified type I collagen can be found, among other places, in Miller et al., Methods In Enzymology 82: 33-64 (1982), Academic Press.

Type II collagen is a homotrimeric collagen comprising three identical α1(II) chains. Purified Type II collagen may be prepared from tissues by, among other methods, the procedure described in Miller et al., Methods In Enzymology, 82: 33-64 (1982), Academic Press.

Type III collagen is a major fibrillar collagen found in skin and vascular tissues. Type III collagen is a homotrimeric collagen comprising three identical α1(III) chains. Methods for purifying type III collagen from tissues can be found in, among other places, Byers et al., Biochemistry 13: 5243-5248 (1974) and Miller et al., Methods in Enzymology 82: 33-64 (1982), Academic Press.

Type IV collagen is found in basement membranes in the form of a sheet rather than fibrils. The most common form of type IV collagen contains two α1(IV) chains and one α2(IV) chain. The particular chains comprising type IV collagen are tissue-specific. Type IV collagen may be purified by, among other methods, the procedures described in Furuto et al., Methods in Enzymology 144: 41-61 (1987), Academic Press.

Type V collagen is a fibrillar collagen found in, primarily, bones, tendon, cornea, skin, and blood vessels. Type V collagen exists in both homotrimeric and heterotrimeric forms. One type of type V collagen is a heterotrimer of two α1(V) chains and α2(V). Another type of type V collagen is a heterotrimer of α1(V), α2(V), and a3(V). Yet another type of type V collagen is a homotrimer of α1(V). Methods for isolating type V collagen from natural sources can be found, among other places, in Elstrow et al., Collagen Rel. Res. 3:181-193 (1983) and Abedin et al., Biosci. Rep. 2: 493-502 (1982).

Type VI collagen has a small triple helical region and two large non-collagenous remainder portions. Type VI collagen is a heterotrimer comprising α1(VI), α2(VI), and α3(VI) chains. Type VI collagen is found in many connective tissues. Descriptions of how to purify type VI collagen from natural sources can be found, among other places, in Wu et al., Biochem. J. 248: 373-381 (1987), and Kielty, et al., J. Cell Sci. 99: 797-807.

Type VII collagen is a fibrillar collagen found in particular epithelial tissues. Type VII is a homotrimeric molecule of three α1(VII) chains. Descriptions of how to purify type VII collagen from tissue can be found in, among other places, Lundstrom et al., J. Biol. Chem. 261: 9042-9048 (1986), and Bentz et al., Proc. Natl. Acad. Sci. USA 80: 3168-3172 (1983).

Type VIII collagen can be found in Descemet's membrane in the cornea. Type VIII collagen is a heterotrimer comprising two α1(VIII) chains and one α2(VIII) chain, although other chain compositions have been reported. Methods for the purification of type VIII collagen from nature can be found, among other places, in Benya et al., J. Biol. Chem. 261: 4160-4169 (1986), and Kapoor et al., Biochemistry 25: 3930-3937 (1986).

Type IX collagen is a fibril associated collagen which can be found in cartilage and vitreous humor. Type IX collagen is a heterotrimeric molecule comprising α1(IX), α2(IX), and α3 (IX) chains. Procedures for purifying type IX collagen can be found, among other places, in Duance, et al., Biochem. J. 221: 885-889 (1984), Ayad et al., Biochem. J. 262: 753-761 (1989), Grant et al., The Control of Tissue Damage, Glauert, A. M., Ed., El Sevier, Amsterdam, pp. 3-28 (1988).

Type X collagen is a homotrimeric compound of α1(X) chains. Type X collagen has been isolated from, among other tissues, hypertrophic cartilage found in growth plates.

Type XI collagen can be found in cartilaginous tissues associated with type II and type IX collagens, as well as other locations in the body. Type XI collagen is a heterotrimeric molecule comprising α1(XI), α2(XI), and α3(XI) chains. Methods for purifying type XI collagen can be found, among other places, in Grant et al., In The Control of Tissue Damage, Glauert, A. M., ed., El Savier, Amsterdam, pp. 3-28 (1988).

Type XII collagen is a fibril associated collagen found primarily associated with type I collagen. Type XII collagen is a homotrimeric molecule comprising three .beta.1(XII) chains. Methods for purifying type XII collagen and variants thereof can be found, among other places, in Dublet et al., J. Biol. Chem. 264: 13150-13156 (1989), Lundstrum et al., J. Biol. Chem. 267: 20087-20092 (1992), Watt et al., J. Biol. Chem. 267: 20093-20099 (1992).

Type XIII is a non-fibrillar collagen found, among other places, in skin, intestine, bone, cartilage, and striated muscle. A detailed description of the type XIII collagen may be found, among other places, in Juvonen et al. J. Biol. Chem. 267: 24700-24707 (1992).

Type XIV is a fibril associated collagen. Type XIV collagen is a homotrimeric molecule comprising three α1(XIV) chains. Methods for isolating type XIV collagen can be found, among other places, in Aubert-Foucher et al., J. Biol. Chem. 266: 19759-19764. (1992) and Watt et al., J. Biol. Chem. 267: 20093-20099 (1992).

Type XV collagen is homologous in structure to type XVIII collagen. Information about the structure and isolation of natural type XV collagen can be found, among other places, in Myers et al., Proc. Natl. Acad. Sci. USA 89: 10144-10148 (1992), Huebner et al., Genomics 14: 220-224 (1992), Kivirikko et al., J. Biol. Chem. 269: 4773-4779 (1994), and Muragaki, J. Biol. Chem. 264: 4042-4046 (1994).

Type XVI collagen is a fibril associated collagen, found in skin, lung fibroblast, keratinocytes, and elsewhere. Information on the structure of type XVI collagen and the gene encoding type XVI can be found, among elsewhere, in Pan et al., Proc. Natl. Acad. Sci. USA 1989: 6565-6569 (1992), and Yamaguchi et al., J. Biochem. 112: 856-863 (1992).

Type XVII collagen is a hemidesmosal transmembrane collagen. Information on the structure of type XVII collagen the gene encoding type XVII collagen can be found, among elsewhere, in Li et al., J. Biol. Chem. 268(12): 8825-8834 (1993), and McGrath et al., Nat. Genet. 11(1): 83-86 (1995).

Type XVIII collagen is similar in structure to type XV collagen and can be isolated from the liver. Descriptions of the structures and isolation of type XVIII collagen from natural sources can be found, among other places, in Rehn et al., Proc. Natl. Acad. Sci. USA 91: 4234-4238 (1994), Oh et al., Proc. Natl. Acad. Sci. USA 91: 4229-4233 (1994), Rehn et al., J. Biol. Chem. 269: 13924-13935 (1994), and Oh et al., Genomics 19: 994-999 (1994).

Type XIX collagen's gene structure Classify it as another member of the FACIT collagenous family. Type XIX mRNA was recently isolated from rhabdomyosarcoma cell. Descriptions of the structures and isolation of type XIX collagen can be found, among other places, in Inoguchi et al., J. Biochem. 117: 137-146 (1995), Yoshioka et al., Genomics 13: 884-886 (1992), Myers et al., J. Biol. Chem. 289: 18549-18557 (1994).

### Non-Helical Domains

Provided herein are polynucleotides encoding polypeptides capable of assembling into triple helices. The triple helical proteins may include non-triple helical domains at the amino and/or carboxy terminal ends or elsewhere. For example, collagen chains often contain both long helical domains and non-helical extensions, or telopeptides. In certain embodiments, the non-helical domains from one type of collagen may be used to replace or modify the non-helical domain of another type of collagen. For example, the collagen II N-terminal domain can be fused to the collagen III triple helical domain.

Further modifications to the non helical domains may be made, including cleavage sites useful for post-translational or post-assembly cleavage of the non-helical domains from the core helical domain.

### Heterologous Sequences

The collagen sequences provided herein may be engineered to contain sequences from other collagen types. Further, the collagen sequences provided herein may be engineered to contain heterologous sites sequence not found in any known collagen. Accordingly, provided herein is an isolated polypeptide comprising an amino acid sequence at least 70% identical to a naturally occurring collagen polypeptide and comprising more than 3, 4, 5, 6, 7, 8, 9 or 10 amino acid modifications, wherein the amino acid modifications can include a protease recognition site, an integrin binding site; a kinase target site; growth factor binding site, cell binding site, and chemical attachment site. Such heterologous sites may be located at the N-terminus, C-terminus, or in the core helical domain of the collagen sequence.

### Molecular Techniques

It will be understood by those skilled in the art that any of a variety of known molecular techniques can be utilized in order to arrive at polynucleotides and polypeptides which are based upon the nucleotide sequences provided herein. Accordingly, a polynucleotide having any desired nucleotide sequence can be prepared by methods known in the art. For example, a polynucleotide containing a nucleotide sequence provided herein can be prepared by known methods, such as, for example, assembly of overlapping oligonucleotides which can be solid phase synthesized, as is described in U.S. Pat. No. 7,262,031, and U.S. Patent Publication Numbers 2005/0106590 and 2007/0009928. The prepared polynucleotide can then be amplified by PCR methodologies or by insertion into a vector, transformation into cells, and subsequent harvesting of the vector from the cells. Examples of such methods for amplification of a polynucleotide are provided in Ausubel et al., 2008, Current Protocols in Molecular Biology, Greene Publishing Associates and Wiley Interscience, N.Y. The polynucleotide itself or amplicon thereof can be inserted into an expression vector configured to produce the polypeptide encoded by the inserted polynucleotide. The expression vector is then inserted into cells, and according to the expression vector used, the cells are treated under conditions suitable for polypeptide expression. Any of a variety of expression vectors, cell types, and polypeptide expression methodologies known in the art can be used, and examples of such methodologies are provided in Ausubel, supra. The expressed polypeptide can be analyzed and manipulated as desired. For example, the expressed polypeptide can be analyzed by Western blot analysis using a known antibody to the expressed polypeptide or using an anti-polypeptide antibody generated by known methods. The expressed polypeptide also can be subjected to one or more purification steps to increase the purity of the expressed polypeptide. Various analytical and purification methods, as well as antibody-generation methods are known in the art, as exemplified in Ausubel, supra.

In some embodiments, the sequence of the polynucleotide can be generated, optionally in conjunction with optimization of a plurality of parameters where one such parameter can be codon pair usage, where the resultant polynucleotide can be prepared by assembly of a plurality of oligonucleotides sufficiently small to be synthesized by known oligonucleotide synthetic methods. Methods known in the art for optimizing multiple parameters in synthetic nucleotide sequences can be applied to optimizing the parameters recited in the present claims. Such methods may advantageously include those exemplified in U.S. Patent App. Publication No. 2005/0106590, U.S. Patent App. Publication No. 2007/0009928, and R. H. Lathrop et al. “Multi-Queue Branch-and-Bound Algorithm for Anytime Optimal Search with Biological Applications” in Proc. Intl. Conf. on Genome Informatics, Tokyo, Dec. 17-19, 2001 pp. 73-82; in Genome Informatics 2001 (Genome Informatics Series No. 12), Universal Academy Press, which are incorporated herein by reference in their entireties. Briefly, in addition to optimizing the various parameters, an exemplary method for generating a sequence can also include dividing the desired sequence into a plurality of partially overlapping segments; optimizing the melting temperatures of the overlapping regions of each segment to disfavor hybridization to the overlapping segments which are non-adjacent in the desired sequence; allowing the overlapping regions of single stranded segments which are adjacent to one another in the desired sequence to hybridize to one another under conditions which disfavor hybridization of non-adjacent segments; and filling in, ligating, or repairing the gaps between the overlapping regions, thereby forming a double-stranded DNA with the desired sequence. This process can be performed manually or can be automated, e.g., in a general purpose digital computer. In one embodiment, the search of possible codon assignments is mapped into an anytime branch and bound computerized algorithm developed for biological applications.

### Optimization of Codon Usage and Translational Kinetics

Methods provided herein for sequence design can also take into consideration parameters such as optimization of codon usage and codon pair usage in the host organism. Thus, the nucleotide sequences encoding the collagen-like polypeptide can be modified to generate sequences optimized for expression in human cells without altering the encoded polypeptide sequences. Computer algorithms are available for codon optimization. For example, web-based algorithms (e.g., Sharp et al. (1988) Nucleic Acids Res. 16:8207-11, hereby incorporated by reference) can be used to generate a nucleotide sequence with optimized expression in a suitable host (e.g., E. coli, S. cerevisiae, P. pastoris, K. lactis, K. marxianus, horse, human, rodent or insect cells).

Further, translational kinetics properties can be optimized, including codon pair usage in the host organism. For example, sequence modifications can be made to place or prevent restriction sites in the sequence, eliminate strong RNA secondary structures and avoid inadvertent Shine-Delgamo sequences. Additionally, methods provided herein include modifying the translational kinetics of a polypeptide-encoding nucleic sequence by removing, preserving, and/or inserting translational pauses into the polypeptide-encoding nucleic sequence. Exemplary methods are set forth in WO 07/130,606 and WO 07/130,650 (each of which is incorporated by reference in its entirety). These references describe methods for calculating codon pair translational kinetics values, creating a synthetic gene for expression in a host organism, and providing codon pair translational kinetic values.

Methods of Inserting Polynucleotide into Vector, Transforming Cells, Expressing Polynucleotide, and Purifying Polypeptide

The nucleic acid sequences provided herein can be present in a polynucleotide (e.g., DNA or RNA molecule). Thus, in one embodiment, provided are polynucleotides containing the nucleic acid sequences provided herein. The polynucleotides can be inserted into a replicable vector for cloning (e.g., amplification of the DNA) or for expression. Various vectors are publicly available and are known in the art. The vector can, for example, be in the form of a plasmid, cosmid, viral particle, or phage. The appropriate nucleic acid sequence can be inserted into the vector by any of a variety of procedures known in the art. Typically, DNA is inserted into an appropriate restriction endonuclease site(s) using techniques known in the art or the DNA is inserted by any of a variety of PCR methodologies. Vector components can generally include, but are not limited to, one or more of a signal sequence, an origin of replication, one or more marker genes, an enhancer element, a promoter, and a transcription termination sequence. Construction of suitable vectors containing one or more of these components employs standard ligation techniques which are known to the skilled artisan.

The encoded polypeptide can be produced recombinantly not only directly, but also as a fusion polypeptide with a heterologous polypeptide, which can be, e.g., a signal sequence or other polypeptide having a specific cleavage site at the N-terminus of the mature protein or polypeptide. In general, the signal sequence can be a component of the vector, or it can be a part of the polynucleotide that is inserted into the vector. The signal sequence can be a prokaryotic signal sequence selected, for example, from the group of the alkaline phosphatase, penicillinase, 1 pp, or heat-stable enterotoxin II leaders. For yeast secretion the signal sequence can be, e.g., the yeast invertase leader, alpha factor leader (including Saccharomyces and Kluyveromyces α-factor leaders, the latter described in U.S. Pat. No. 5,010,182), or acid phosphatase leader, the C. albicans glucoamylase leader (EP 362,179 published 4 Apr. 1990), or the signal described in WO 90/13646 published 15 Nov. 1990. In mammalian cell expression, mammalian signal sequences can be used to direct secretion of the protein, such as signal sequences from secreted polypeptides of the same or related species, as well as viral secretory leaders.

Both expression and cloning vectors contain a polynucleotide that permits the vector to replicate in one or more selected host cells. Such sequences are well known for a variety of bacteria, yeast, and viruses. The origin of replication from the plasmid pBR322 is suitable for most Gram-negative bacteria, the 2μ plasmid origin is suitable for yeast, and various viral origins (SV40, polyoma, adenovirus, VSV or BPV) are useful for cloning vectors in mammalian cells.

Expression and cloning vectors will typically contain a selection gene, also termed a selectable marker. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media, e.g., the gene encoding D-alanine racemase for Bacilli.

An example of suitable selectable markers for mammalian cells are those that enable the identification of cells competent to take up the polynucleotide-containing vector, such as DHFR or thymidine kinase. An appropriate host cell when wild-type DHFR is employed is the CHO cell line deficient in DHFR activity, prepared and propagated as described by Urlaub et al., Proc. Natl. Acad. Sci. USA, 77:4216 (1980). A suitable selection gene for use in yeast is the trp1 gene present in the yeast plasmid YRp7 [Stinchcomb et al., Nature, 282:39 (1979); Kingsman et al., Gene, 7:141 (1979); Tschemper et al., Gene, 10:157 (1980)]. The trp1 gene provides a selection marker for a mutant strain of yeast lacking the ability to grow in tryptophan, for example, ATCC No. 44076 or PEP4-1 [Jones, Genetics, 85:12 (1977)].

Expression and cloning vectors usually contain a promoter operably linked to the polynucleotide provided herein to direct mRNA synthesis. Promoters recognized by a variety of potential host cells are well known. Promoters suitable for use with prokaryotic hosts include the β-lactamase and lactose promoter systems [Chang et al., Nature, 275:615 (1978); Goeddel et al., Nature, 281:544 (1979)], alkaline phosphatase, a tryptophan (trp) promoter system [Goeddel, Nucleic Acids Res., 8:4057 (1980); EP 36,776], and hybrid promoters such as the tac promoter [deBoer et al., Proc. Natl. Acad. Sci. USA, 80:21-25 (1983)]. Promoters for use in bacterial systems also will contain a Shine-Dalgarno (S.D.) sequence operably linked to the polynucleotide provided herein.

Examples of suitable promoting sequences for use with yeast hosts include the promoters for 3-phosphoglycerate kinase [Hitzeman et al., J. Biol. Chem., 255:2073 (1980)] or other glycolytic enzymes [Hess et al., J. Adv. Enzyme Reg., 7:149 (1968); Holland, Biochemistry, 17:4900 (1978)], such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase.

Other yeast promoters, which are inducible promoters having the additional advantage of transcription controlled by growth conditions, are the promoter regions for alcohol dehydrogenase 2, isocytochrome C, acid phosphatase, degradative enzymes associated with nitrogen metabolism, metallothionein, glyceraldehyde-3-phosphate dehydrogenase, and enzymes responsible for maltose and galactose utilization. Suitable vectors and promoters for use in yeast expression are further described in EP 73,657.

Transcription from vectors in mammalian host cells is controlled, for example, by promoters obtained from the genomes of viruses such as polyoma virus, fowlpox virus (UK 2,211,504 published 5 Jul. 1989), adenovirus (such as Adenovirus 2), bovine papilloma virus, avian sarcoma virus, cytomegalovirus, a retrovirus, hepatitis-B virus and Simian Virus 40 (SV40), from heterologous mammalian promoters, e.g., the actin promoter or an immunoglobulin promoter, and from heat-shock promoters, provided such promoters are compatible with the host cell systems.

Transcription by higher eukaryotes can be increased by inserting an enhancer sequence into the vector. Enhancers are cis-acting elements of DNA, usually about from 10 to 300 bp, that act on a promoter to increase its transcription. Many enhancer sequences are now known from mammalian genes (globin, elastase, albumin, α-fetoprotein, and insulin). Typically, however, one will use an enhancer from a eukaryotic cell virus. Examples include the SV40 enhancer on the late side of the replication origin (bp 100-270), the cytomegalovirus early promoter enhancer, the polyoma enhancer on the late side of the replication origin, and adenovirus enhancers. The enhancer can be spliced into the vector at a position 5′ or 3′ to the polynucleotide provided herein, but is preferably located at a site 5′ from the promoter.

Expression vectors used in eukaryotic host cells (yeast, fungi, insect, plant, animal, human, or nucleated cells from other multicellular organisms) will also contain sequences necessary for the termination of transcription and for stabilizing the mRNA. Such sequences are commonly available from the 5′ and, occasionally 3′, untranslated regions of eukaryotic or viral DNAs or cDNAs. These regions contain nucleotide segments transcribed as polyadenylated fragments in the untranslated portion of the mRNA transcribed from the polynucleotide provided herein.

Still other methods, vectors, and host cells suitable for adaptation to the synthesis of the encoded proteins in recombinant vertebrate cell culture are described in Gething et al., Nature, 293:620-625 (1981); Mantei et al., Nature, 281:40-46 (1979); EP 117,060; and EP 117,058.

Host cells are transfected or transformed with expression or cloning vectors described herein for polypeptide production and cultured in conventional nutrient media modified as appropriate for inducing promoters, selecting transformants, or amplifying the genes encoding the desired sequences. The culture conditions, such as media, temperature, pH and the like, can be selected by the skilled artisan without undue experimentation. In general, principles, protocols, and practical techniques for maximizing the productivity of cell cultures can be found in Mammalian Cell Biotechnology: a Practical Approach, M. Butler, ed. (IRL Press, 1991) and Sambrook et al., supra.

Methods of eukaryotic cell transfection and prokaryotic cell transformation are known to the ordinarily skilled artisan, for example, CaCl₂, CaPO₄, liposome-mediated and electroporation. Depending on the host cell used, transformation is performed using standard techniques appropriate to such cells. The calcium treatment employing calcium chloride, as described in Sambrook et al., supra, or electroporation is generally used for prokaryotes. Infection with Agrobacterium tumefaciens is used for transformation of certain plant cells, as described by Shaw et al., Gene, 23:315 (1983) and WO 89/05859 published 29 June 1989. For mammalian cells without such cell walls, the calcium phosphate precipitation method of Graham and van der Eb, Virology, 52:456-457 (1978) can be employed. General aspects of mammalian cell host system transfections have been described in U.S. Pat. No. 4,399,216. Transformations into yeast are typically carried out according to the method of Ito, H.; Fukuda, Y.; Murata, K.; Kimura, A. Transformation of Intact Yeast-Cells Treated with Alkali Cations. J. Bacteriol. 1983, 153:163-168. However, other methods for introducing DNA into cells, such as by nuclear microinjection, electroporation, bacterial protoplast fusion with intact cells, or polycations, e.g., polybrene, polyomithine, can also be used. For various techniques for transforming mammalian cells, see Keown et al., Methods in Enzymology, 185:527-537 (1990) and Mansour et al., Nature, 336:348-352 (1988).

Suitable host cells for cloning or expressing the DNA in the vectors herein include prokaryote, yeast, or higher eukaryote cells. Suitable prokaryotes include but are not limited to eubacteria, such as Gram-negative or Gram-positive organisms, for example, Enterobacteriaceae such as E. coli. Various E. coli strains are publicly available, such as E. coli K12 strain MM294 (ATCC 31,446); E. coli X1776 (ATCC 31,537); E. coli strain W3110 (ATCC 27,325) and K5 772 (ATCC 53,635). Other suitable prokaryotic host cells include Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis (e.g., B. licheniformis 41P disclosed in DD 266,710 published 12 Apr. 1989), Pseudomonas such as P. aeruginosa, and Streptomyces. These examples are illustrative rather than limiting. Strain W3110 is one particularly preferred host or parent host because it is a common host strain for recombinant DNA product fermentations. Preferably, the host cell secretes minimal amounts of proteolytic enzymes. For example, strain W3110 can be modified to effect a genetic mutation in the genes encoding proteins endogenous to the host, with examples of such hosts including E. coli W3110 strain 1A2, which has the complete genotype tonA; E. coli W3110 strain 9E4, which has the complete genotype tonA ptr3; E. coli W3110 strain 27C7 (ATCC 55,244), which has the complete genotype tonA ptr3 phoA E15 (argF-lac)169 degP ompT kanr; E. coli W3110 strain 37D6, which has the complete genotype tonA ptr3 phoA E15 (argF-lac)169 degP ompT rbs7 ilvG kanr; E. coli W3110 strain 40B4, which is strain 37D6 with a non-kanamycin resistant degP deletion mutation; and an E. coli strain having mutant periplasmic protease disclosed in U.S. Pat. No. 4,946,783 issued 7 Aug. 1990. Alternatively, in vitro methods of cloning, e.g., PCR or other nucleic acid polymerase reactions, are suitable.

In addition to prokaryotes, eukaryotic microbes such as filamentous fungi or yeast are suitable cloning or expression hosts for polynucleotide-containing vectors. Saccharomyces cerevisiae is a commonly used lower eukaryotic host microorganism. Others include Schizosaccharomyces pombe (Beach and Nurse, Nature, 290: 140 [1981]; EP 139,383 published 2 May 1985); Kluyveromyces hosts (U.S. Pat. No. 4,943,529; Fleer et al., Bio/Technology, 9:968-975 (1991)) such as, e.g., K. lactis (MW98-8C, CBS683, CBS4574; Louvencourt et al., J. Bacteriol., 154(2):737-742 [1983]), K. fragilis (ATCC 12,424), K. bulgaricus (ATCC 16,045), K. wickeramii (ATCC 24,178), K. waltii (ATCC 56,500), K. drosophilarum (ATCC 36,906; Van den Berg et al., Bio/Technology, 8:135 (1990)), K. thermotolerans, and K. marxianus; yarrowia (EP 402,226); Pichia pastoris (EP 183,070; Sreekrishna et al., J. Basic Microbiol., 28:265-278 [1988]); Candida; Trichoderma reesia (EP 244,234); Neurospora crassa (Case et al., Proc. Natl. Acad. Sci. USA, 76:5259-5263 [1979]); Schwanniomyces such as Schwanniomyces occidentalis (EP 394,538 published 31 Oct. 1990); and filamentous fungi such as, e.g., Neurospora, Penicillium, Tolypocladium (WO 91/00357 published 10 Jan. 1991), and Aspergillus hosts such as A. nidulans (Ballance et al., Biochem. Biophys. Res. Commun., 112:284-289 [1983]; Tilburn et al., Gene, 26:205-221 [1983]; Yelton et al., Proc. Natl. Acad. Sci. USA, 81: 1470-1474 [1984]) and A. niger (Kelly and Hynes, EMBO J., 4:475-479 [1985]). Methylotropic yeasts are suitable herein and include, but are not limited to, yeast capable of growth on methanol selected from the genera consisting of Hansenula, Candida, Kloeckera, Pichia, Saccharomyces, Torulopsis, and Rhodotorula. A list of specific species that are exemplary of this class of yeasts can be found in C. Anthony, The Biochemistry of Methylotrophs, 269 (1982).

Suitable host cells for the expression of glycosylated polypeptides are derived from multicellular organisms. Examples of invertebrate cells include insect cells such as Drosophila S2 and Spodoptera Sf9, as well as plant cells. Examples of useful mammalian host cell lines include Chinese hamster ovary (CHO) and COS cells. More specific examples include monkey kidney CV1 line transformed by SV40 (COS-7, ATCC CRL 1651); human embryonic kidney line (293 or 293 cells subcloned for growth in suspension culture, Graham et al., J. Gen Virol., 36:59 (1977)); Chinese hamster ovary cells/-DHFR (CHO, Urlaub and Chasin, Proc. Natl. Acad. Sci. USA, 77:4216 (1980)); mouse sertoli cells (TM4, Mather, Biol. Reprod., 23:243-251 (1980)); human lung cells (W138, ATCC CCL 75); human liver cells (Hep G2, HB 8065); and mouse mammary tumor (MMT 060562, ATCC CCL51). The selection of the appropriate host cell is deemed to be within the skill in the art.

During their biosynthesis, collagens undergo various post-translational modifications (Van der Rest et al., Adv. Mol. Cell. Biol. 6: 1-67 (1993)). For example, the proline residues of collagen are hydroxylated into 4-hydroxyproline, thereby contributing to the stability of collagen by allowing the formation of additional interchain hydrogen bonds. The enzyme catalyzing this modification is prolyl 4-hydroxylase (Kivirikko et al., Post-translational modifications of proteins (Harding, J. J., Crabbe, M. J. C., eds) pp. 1-51, CRC Press, Boca Raton, Fla. (1992)). As further example, the N-propeptide and C-propeptide comprising the collagen precursor molecule, “procollagen,” are cleaved during post-translational events by the enzymes N-proteinase and C-proteinase, respectively. Accordingly, in the methods provided herein, where a host cell does not naturally possess the mechanism to correctly post-translationally modify collagen, the collagen polynucleotide can be co-expressed with the enzyme prolyl 4-hydroxylase. For example, where yeast is the host cell, the collagen polynucleotide can be expressed in yeast cells which have been engineered to express prolyl 4-hydroxylase, as described by Toman et al. (J. Biol. Chem., 275:23303 (2000)).

Gene amplification and/or expression can be measured in a sample directly, for example, by conventional Southern blotting, Northern blotting to quantitate the transcription of mRNA [Thomas, Proc. Natl. Acad. Sci. USA, 77:5201 5205 (1980)], dot blotting (DNA analysis), or in situ hybridization, using an appropriately labeled probe, based on the sequences provided herein. Alternatively, antibodies can be employed that can recognize specific duplexes, including DNA duplexes, RNA duplexes, and DNA RNA hybrid duplexes or DNA protein duplexes. The antibodies in turn can be labeled and the assay can be carried out where the duplex is bound to a surface, so that upon the formation of duplex on the surface, the presence of antibody bound to the duplex can be detected.

Gene expression, alternatively, can be measured by immunological methods, such as immunohistochemical staining of cells or tissue sections and assay of cell culture or body fluids, to quantitate directly the expression of gene product. Antibodies useful for immunohistochemical staining and/or assay of sample fluids can be either monoclonal or polyclonal, and can be prepared in any mammal. Conveniently, the antibodies can be prepared against any polypeptide provided herein or against a synthetic peptide based on the sequences provided herein or against exogenous sequence fused to the polypeptide or fragment thereof and encoding a specific antibody epitope.

Polypeptides can be recovered from culture medium or from host cell lysates. If membrane-bound, it can be released from the membrane using a suitable detergent solution (e.g. Triton-X 100) or by enzymatic cleavage. Cells employed in expression of polypeptides can be disrupted by various physical or chemical means, such as freeze-thaw cycling, sonication, mechanical disruption, or cell lysing agents, as is known in the art.

### Purification of Polypeptides

It may be desired to purify polypeptides. The following procedures are exemplary of suitable purification procedures: by fractionation on an ion-exchange column; ethanol precipitation; reverse phase HPLC; chromatography on silica or on a cation-exchange resin such as DEAE; chromatofocusing; SDS-PAGE; ammonium sulfate precipitation; gel filtration using, for example, Sephadex G-75; protein A Sepharose columns to remove contaminants such as IgG; and metal chelating columns to bind epitope-tagged forms of the polypeptide. Various additional known methods of protein purification can be employed; exemplary methods are described in Deutscher, Methods in Enzymology, 182 (1990); Scopes, Protein Purification: Principles and Practice, Springer-Verlag, New York (1982). The purification step(s) selected will depend, for example, on the nature of the production process used and the particular polypeptide produced.

Also provided herein is an expression system, comprising an expression vector in a host organism or an expressible nucleic acid integrated into the chromosome of a host organism, wherein the expression vector or integrated expressible nucleic acid include a DNA sequence of the embodiments provided herein operably linked to an expression control sequence. As used herein, an expression vector is a DNA or RNA vector that is capable of transforming a host cell and of effecting expression of a specified nucleic acid molecule. Typically, the expression vector is also capable of replicating within the host cell. Expression vectors can be either prokaryotic or eukaryotic, and are typically viruses or plasmids. As used herein, an integrated expressible nucleic acid is a DNA that has been incorporated into a host chromosome and contains an exogenous gene in accordance with the nucleic acid molecules provided herein, and further optionally containing regulatory sequences capable of effecting and/or modulating the expression of the exogenous gene.

The term operably linked refers to functional linkage between a nucleic acid expression control sequence (such as a promoter, or array of transcription factor binding sites) and a second nucleic acid sequence, wherein the expression control sequence directs transcription of the nucleic acid corresponding to the second sequence. An operably linked expression vector can also include secretion signals and other modifying sequences, and can encode chaperones and proteins for a variety of organisms and systems.

Also provided herein are methods of expressing a polypeptide-encoding nucleotide sequence generated by the methods provided herein. Methods of expressing polypeptides from polypeptide-encoding nucleotide sequences are known in the art, as exemplified, for example, by the techniques described in Maniatis et al., 1989, Molecular Cloning: A Laboratory Manual, Cold Spring Harbor Laboratory, N.Y. and Ausubel et al., 2008, Current Protocols in Molecular Biology, Greene Publishing Associates and Wiley Interscience, N.Y. The methods include inserting a polypeptide-encoding nucleotide sequence designed by the methods provided herein into a cell, and expressing the polypeptide-encoding nucleotide sequence under conditions suitable for gene expression. Additionally provided expression methods include cell-free expression systems as known in the art, where such methods include providing a polypeptide-encoding nucleotide sequence designed by the methods provided herein and contacting the polypeptide-encoding nucleotide sequence with a cell-free expression system under conditions suitable for protein translation.

In the discussion above, reference has been made to polynucleotides and the nucleotide sequences thereof, and to polypeptides and amino acid sequences thereof. It is specifically contemplated herein that both the polynucleotides/polypeptides and the sequences thereof are equally aspects of the invention, and reference to the polynucleotide/polypeptide equally applies to the sequence thereof, and vice versa.

The following examples exemplify application of the teachings provided herein and should not be construed to limit the scope of the invention. All references referred to herein are expressly incorporated by reference in their entireties.

### EXAMPLES

### Example 1

### Assembly of Genes that Encode Collagen-Like Biopolymers

Design of domains of synthetic genes. Naturally-produced fibril-forming collagens, which include collagens I, II, and III, are formed in mammalian cells as pre-procollagens, with the main features described in a simplified schematic below. The dominant region is the triple-helical domain, which provides structure and is encoded by the Glycine-X-Y repeating sequence, where X is often proline (Pro, P) and Y is often hydroxyproline (Hyp, O). The signal peptide is necessary to direct the protein to the endoplasmic reticulum, and is subsequently cleaved. The function of the N-propeptide has not yet been definitively established, and the region adjacent to and including the C-propeptide domain is important for the initiation of triple-helix formation. Since both the N- and C-propeptides are subsequently cleaved by procollagen peptidases, the final collagen molecule contains the triple-helical domain at a length of approximately 1000 amino acids.

The gene design is based on the regions needed for structural integrity, cell interactions, and production within yeast cells. Specifically they consist of three main domains: (1) N-terminal domain (signal peptide and N-propeptide), (2) structural region for stability, and (3) the C-terminal domain. The DNA sequence of Domains 1 and 3 is optimized for yeast and for PCR-synthesis; these domains are identical for all biopolymers. The sequence and strategy for prescribing variability in Domain 2 is described below. Unique restriction sites between Domains 1 & 2, and 2 & 3, are introduced such that variants of Domain 2 can be PCR-assembled and ligated into a vector containing Domains 1 and 3.

Domain 1: Signal peptide and N-propeptide. Domain 1 consists of a signal peptide to direct the protein to the endoplasmic reticulum of the yeast cell. This sequence is from the native human collagen III gene, which has been shown to be superior to the yeast signals tested. The signal peptide is followed by the N-propeptide of human collagen Ill. Thus, Domain 1 (and Domain 3) are removed by pepsin during subsequent purification procedures. Such treatment has been found to retain the fibrillar nature of collagen.

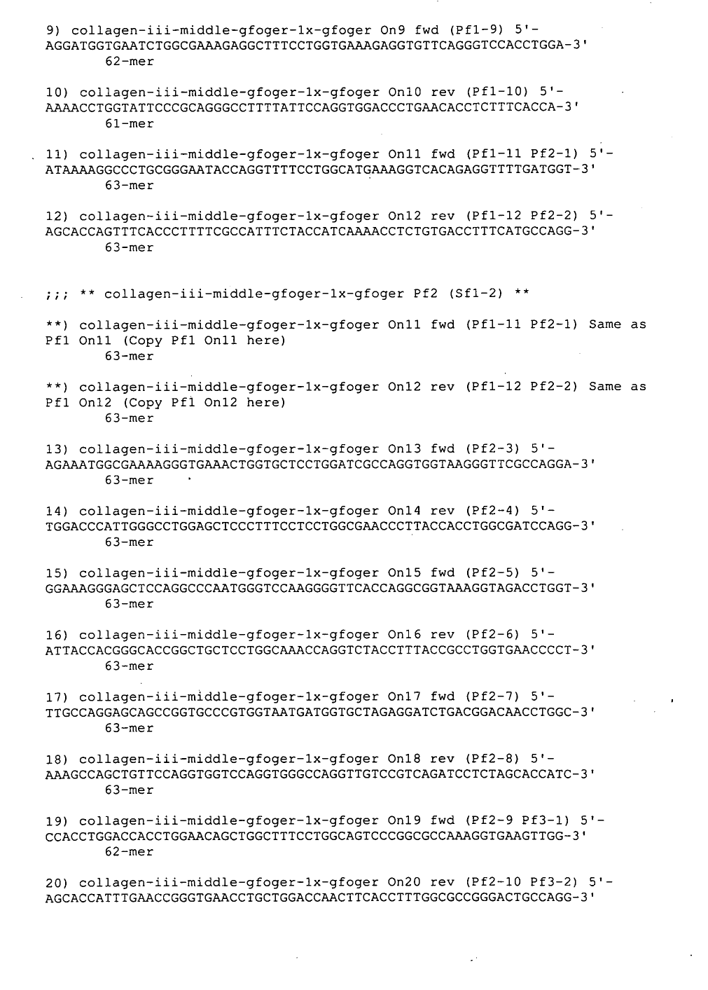

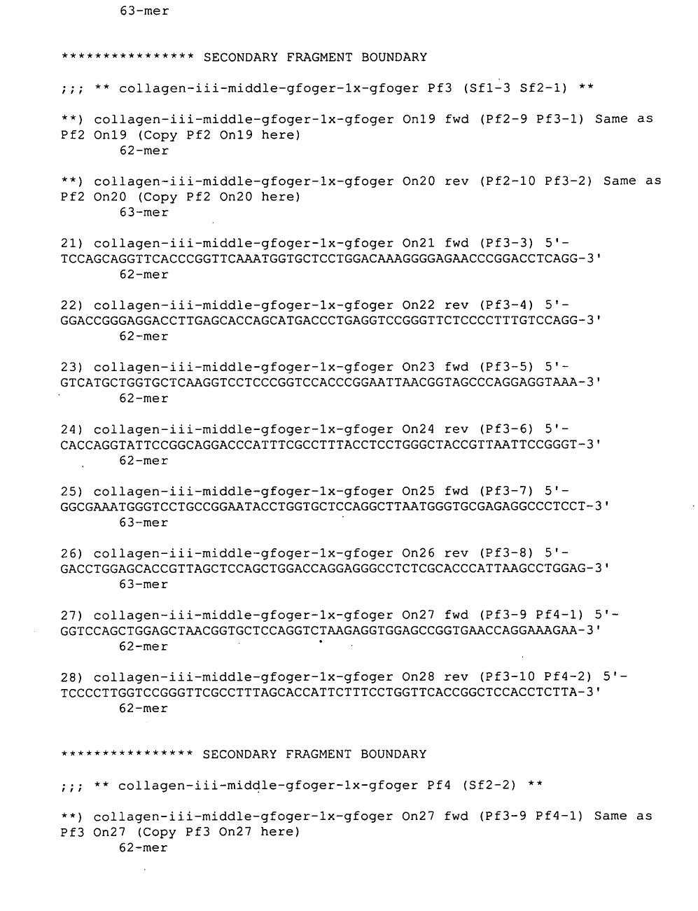

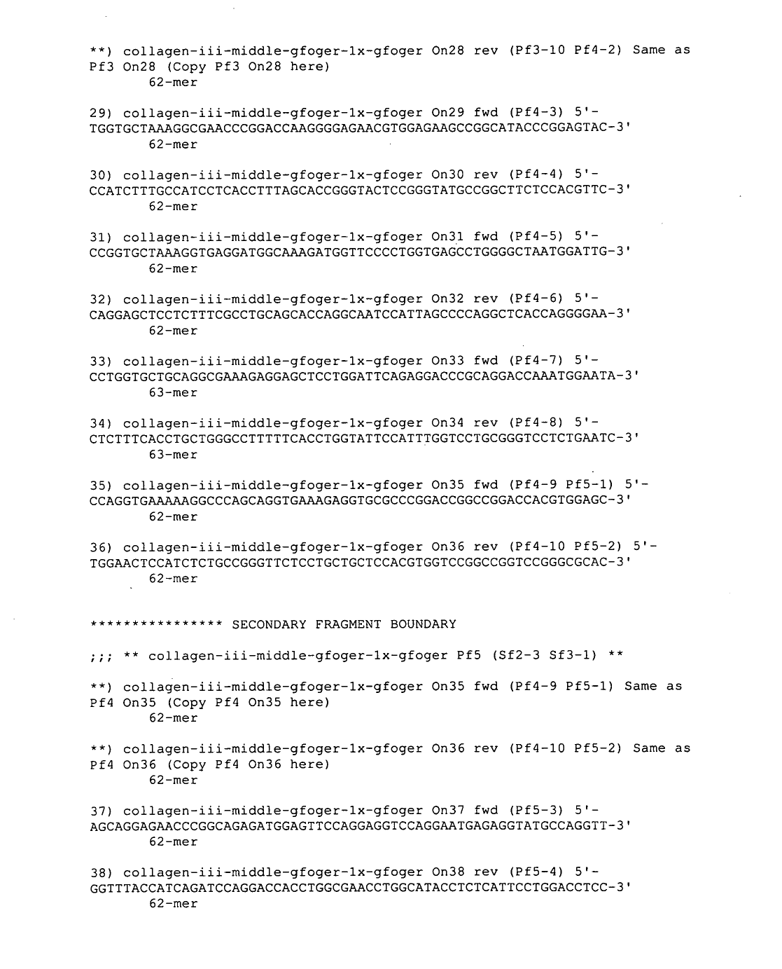

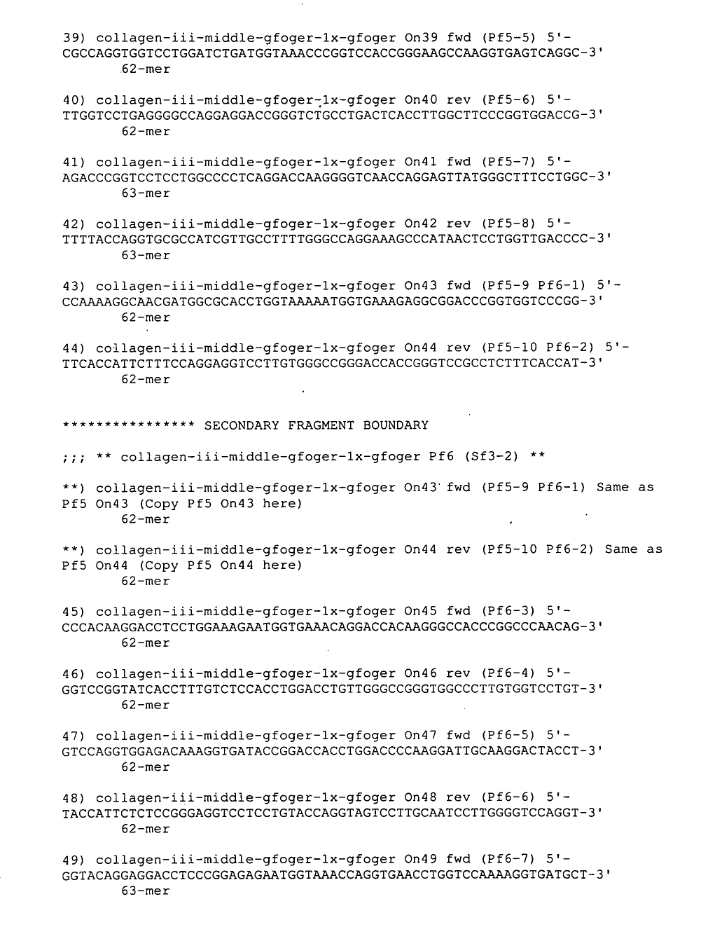

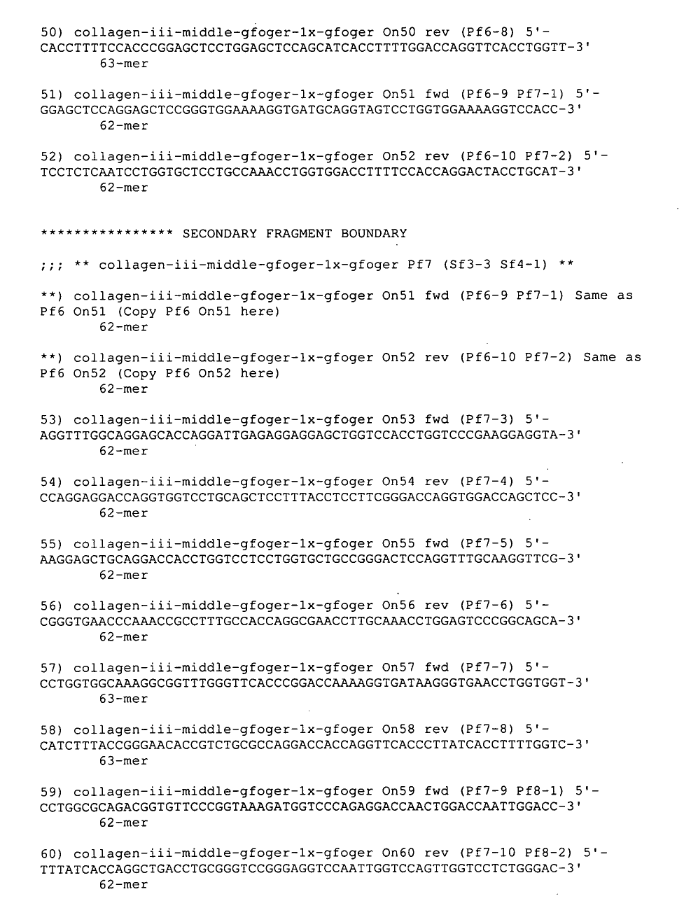

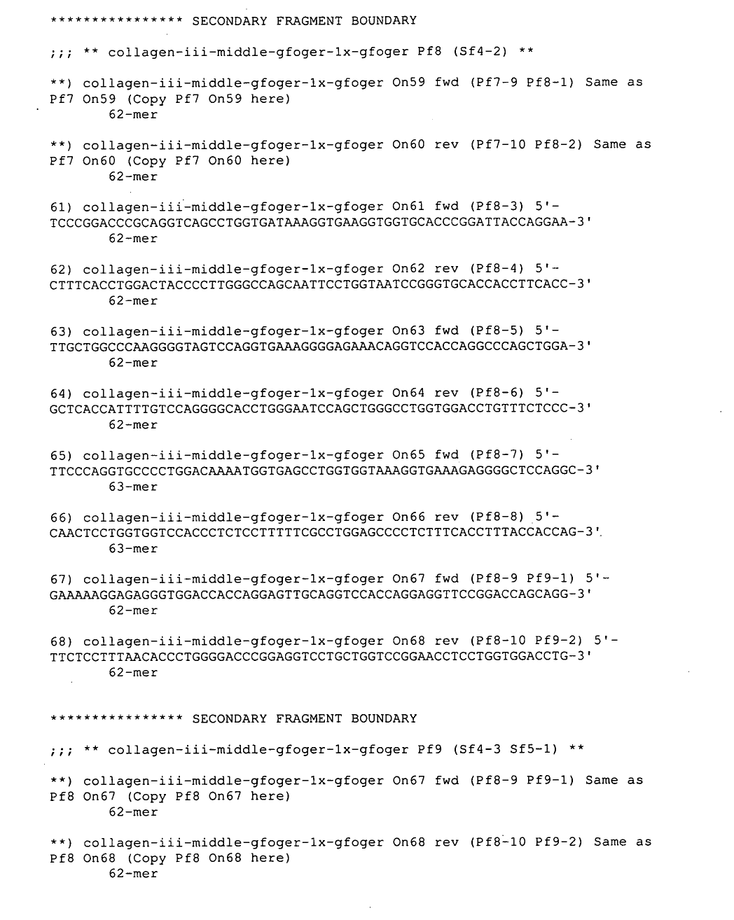

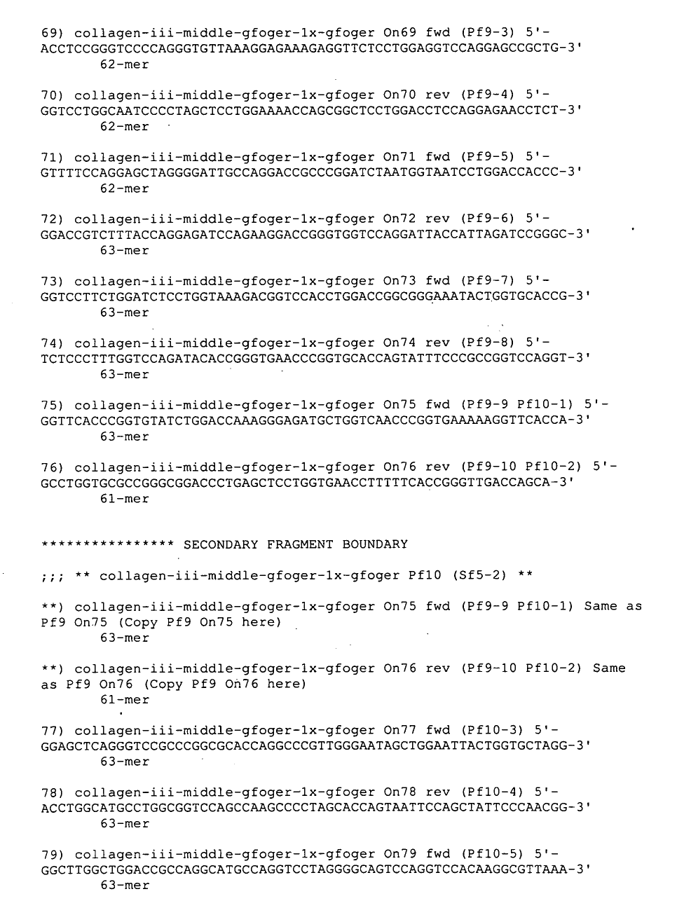

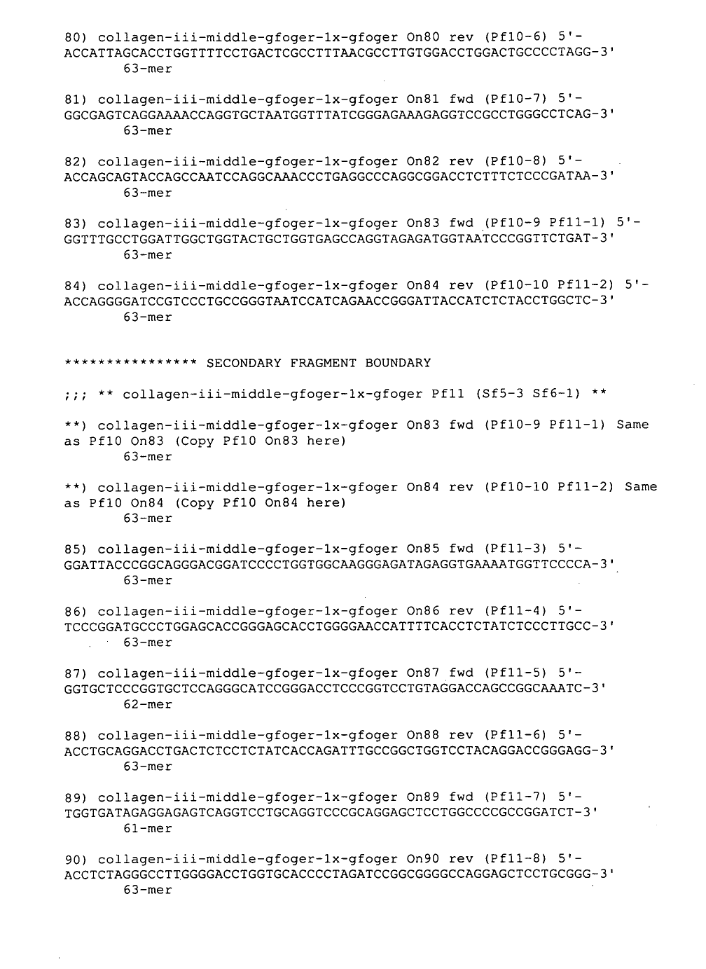

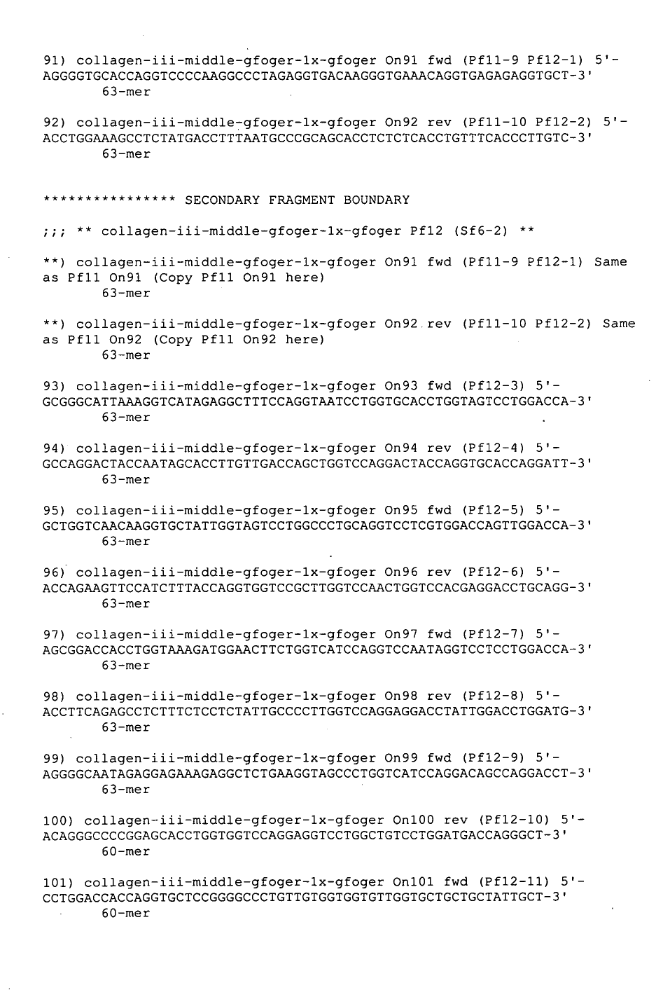

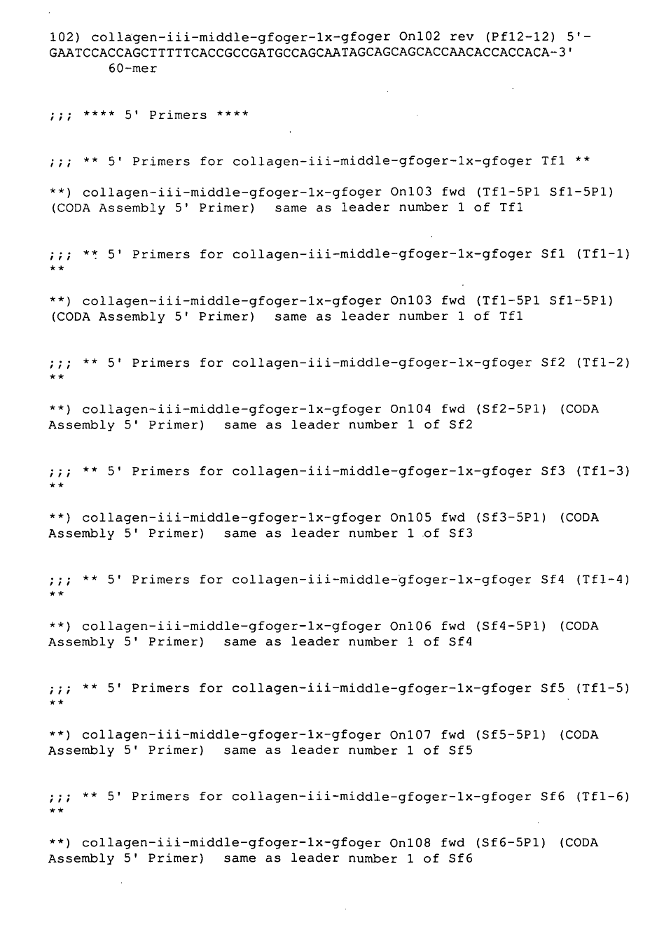

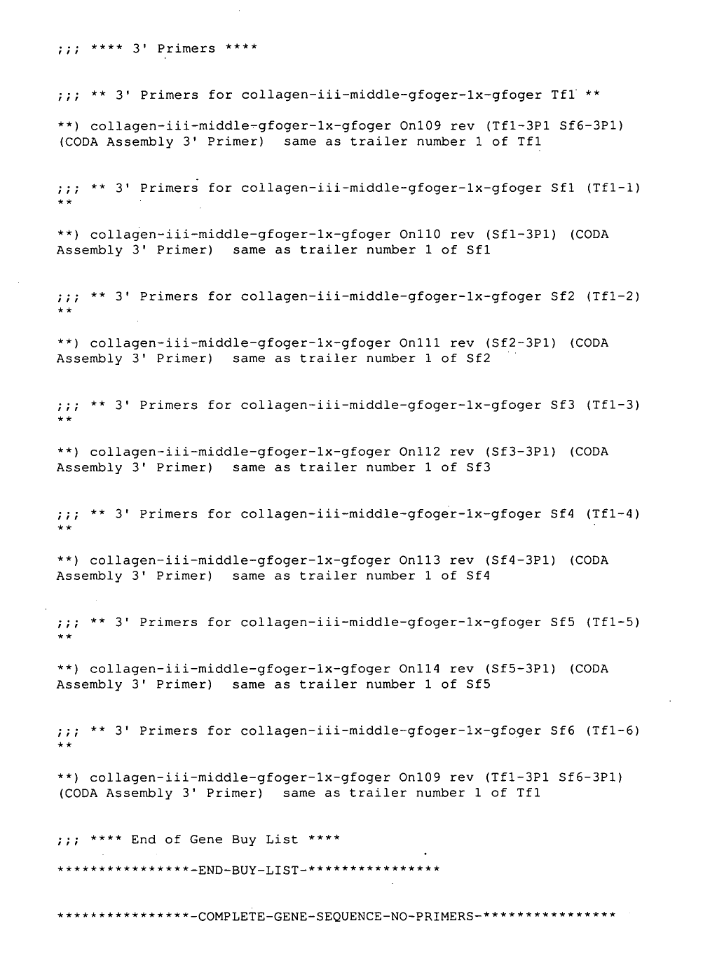

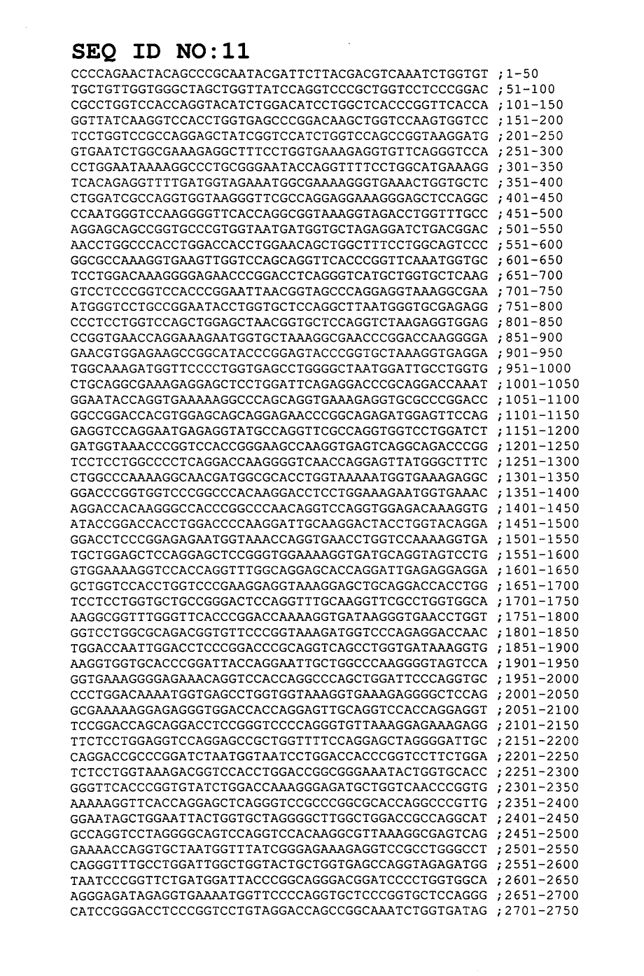

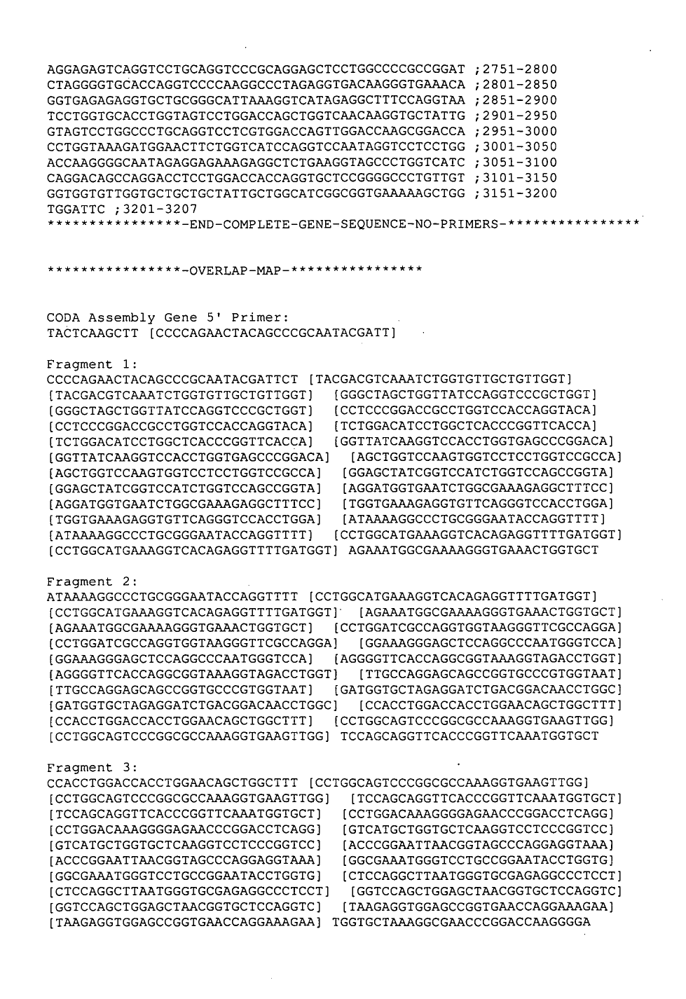

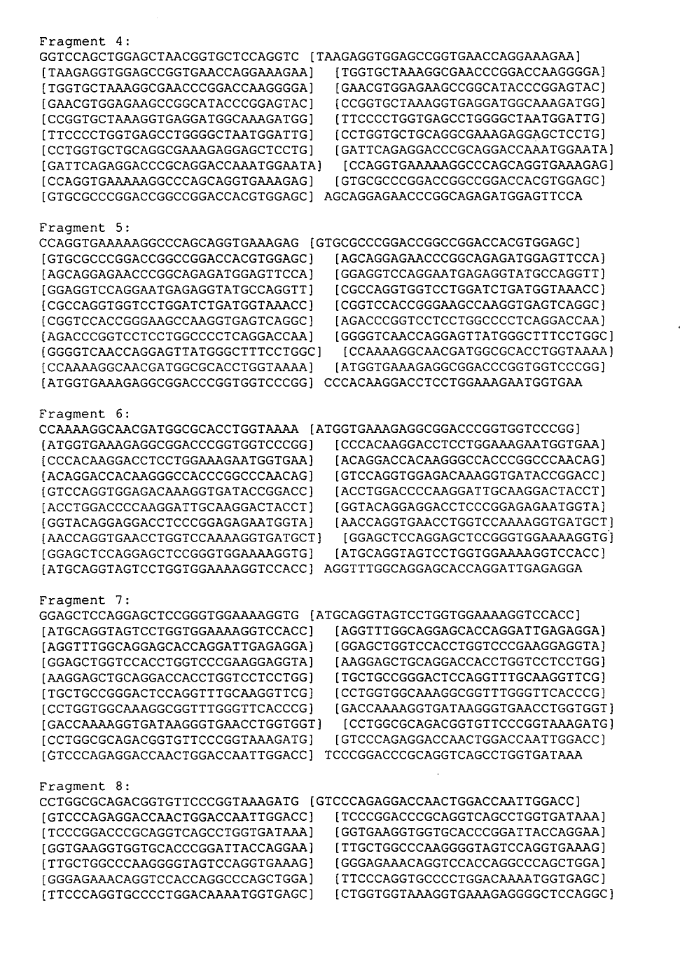

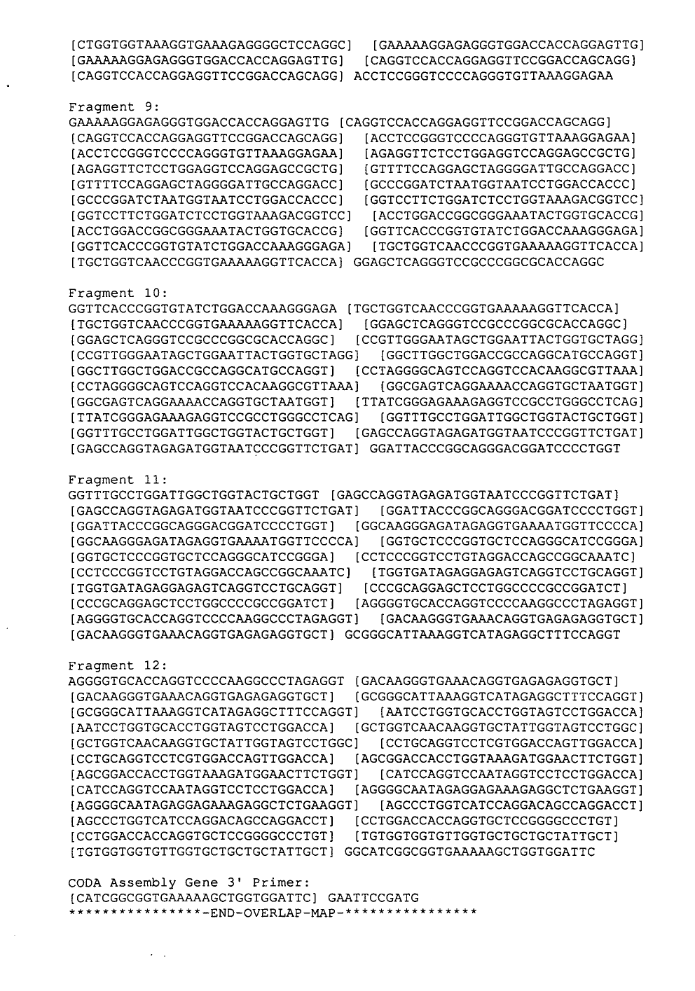

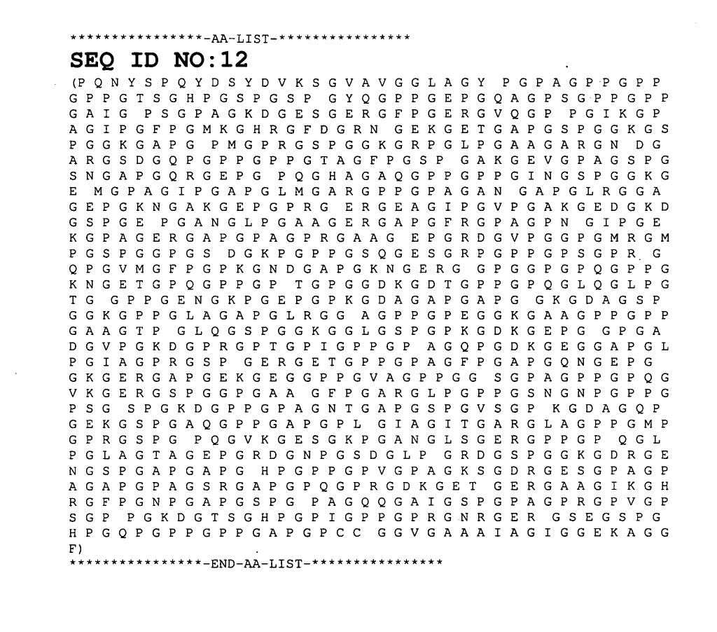

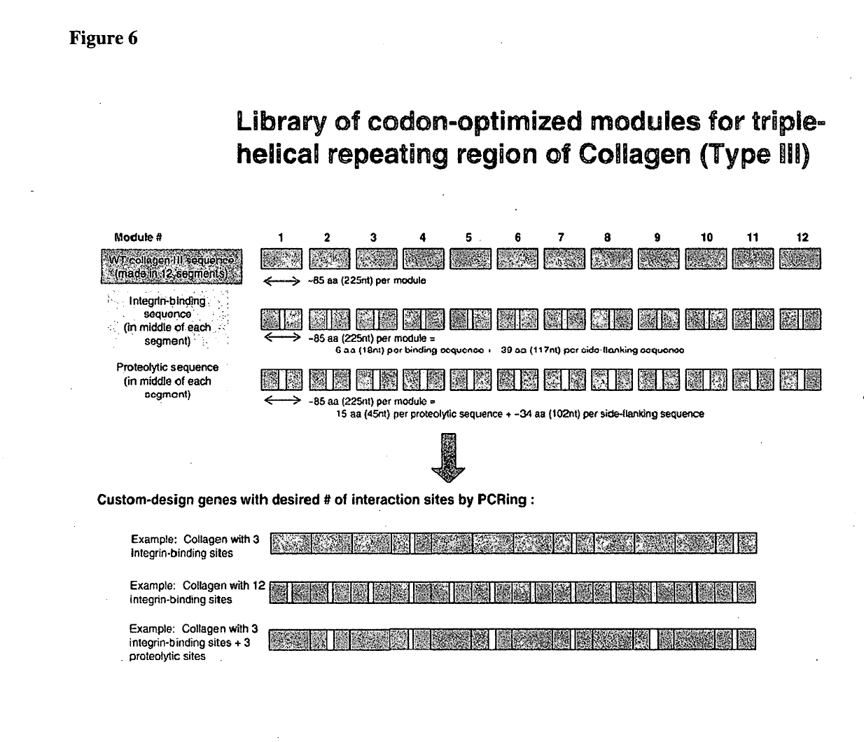

Domain 2: Triple-helical structural region. Domain 2 is the structural domain that displays the repeating Gly-X-Y sequence, and results in a triple-helical scaffold. The native human sequence of collagen III is used as the departure point for the engineered biopolymers. FIG. 6 describes the proposed approach for creating the genes encoding the synthetic polymers and these steps are performed by the methods described hereinabove. In particular, the strategy allows the following: (1) specify different types of cell-binding and cell-interaction sites (e.g., GFOGER & protease sites described below), (2) alter concentrations of each type of site independently from one another, (3) specify the location of different sites within the gene, and (4) produce polymers that retain triplehelical structure.

As described in FIG. 6, three sets of genes are synthesized, each made in 14 overlapping modules. Each of the 14 modules in an individual set is created from PCR assembly of oligonucleotides, and the lengths of each module (˜260 bp or ˜85 amino acids) are within the range for optimal gene assembly. Set 1 encodes for the natural protein sequence of human collagen Ill. Sets 2 & 3 is synthesized as the same 14 modules, with the exception that an integrin-binding GFOGER site (for set 2) or a protease cleavage site (for set 3) is inserted within each fragment's non-overlapping region. Synthesis of specific desired genes can then be performed by PCR assembly, with the annealing temperature defined. In one set, collagen gene containing 6 GFOGER sites is created by mixing modules 3, 5, 7, 9, 11 & 13 from Set 2 with modules 1, 2, 4, 6, 8, 10, 12 & 14 from Set 1.

The modular technology for gene synthesis described hereinabove makes it possible to optimally define the sequence of each module for protein expression in yeast and for in vitro synthesis. In addition, the presence of repeating peptide units, which usually results in the problem of nonspecific annealing during conventional oligonucleotide-based gene synthesis, is addressed by the optimization algorithm. Among other considerations, the algorithm determines all possible codon combinations and their resulting critical melting temperatures.

This strategy, which combines modularity with the ability to define the sequence, enables tremendous flexibility in the creation of the gene. For example, cell-interaction sites that are desired in different locations can be obtained by either using a different module or redefining the sequence of the module, depending on the location needed. Because the introduction of too many foreign sites often disrupts triple-helical assembly, the tolerance for these sites is systematically tested by resynthesizing the gene with a progressively decreasing number of sites until a stable assembly is obtained. This strategy facilitates the making of polymers of different lengths and the introduction of restriction sites into desired regions.

Cell-reactive sequences in Domain 2: GFOGER and protease. The ability to dictate cellular processes with the substrate environment through specific cell-matrix interactions has significant implications in the area of tissue regeneration. By defining characteristics of the substrate matrix, such as stability, mechanical stiffness, and introduction of specific cell-reactive sites, cellular responses can be modulated. Two types of such sequences are incorporated into the collagen-based biopolymers: sites which bind to integrins and substrates for matrix metalloproteinases. Recent studies have shown that for osteoprogenitor cells to penetrate a synthetic polyethylene glycol matrix and regenerate into bone tissue, both the number of RGD-adhesion sites and MMP cleavage sites must be regulated. The concentration of collagen based adhesion and proteolysis sites into the scaffold of a collagen III backbone are independently varied to investigate the ability of the designed biopolymers to modulate cellular responses.

Integrins are the primary receptors on cell surfaces that mediate cell adhesion to the extracellular matrix. Integrin-matrix interactions have been implicated in processes such as cell adhesion, migration, and differentiation, and genetic mutations in these receptors can result in pathological states such as tumor growth, tumor metastasis, muscular dystrophy, and thrombosis. To test the feasibility of the approach to incorporate integrin interactions into a full-length collagen-like substrate which does not naturally have such a sequence, a series of GFOGER (Gly-Phe-Hyp-Gly-Glu-Arg) sequences are introduced into the biopolymers. GFOGER is a binding site found once within natural collagen I (but not in collagen III) and is specifically recognized by several types of integrins. The crystal structures of a GFOGER-containing peptide and integrin a2 μl have been determined to high resolution. They show that the ligand self-assembles into a triple-helical collagen-like structure, and integrin interacts with two of the three collagen-like strands, demonstrating that the triple-helical tertiary structure of collagen is critical for recognition. These types of interactions, therefore, can only be synthetically mimicked in a system that enables correct collagenlike assembly. Relatively short peptides containing the sequence GFOGER, when adsorbed to polystyrene substrate surfaces, have been shown to promote the expression of osteoblast-specific genes in immature osteoblast cells.

In order to determine whether these novel biopolymers can elicit cellular responses such as adhesion, differentiation, and migration, the following investigations are performed. In investigations using synthetic polymers, such effects are known to be modulated by varying the frequency and combination of cell interaction sites within the collagen-like matrix. Thus, the proposed recombinant biopolymers are described in Table 1.

TABLE 1
Summary of proposed synthetic collagen-like polymers (Domain 2).

| | # modules in gene | | |
|---|---|---|---|
| | | | Protease |
| | Native | GFOGER | site |
| Description | (set 1) | (set 2) | (set 3) |
| Control: Native collagen III sequence | 14 | 0 | 0 |
| (gene optimized for yeast) | | | |
| Integrin variant - 3x | 11 | 3 | 0 |
| Integrin variant - 6x | 8 | 6 | 0 |
| Integrin variant - 12x | 2 | 12 | 0 |
| Protease variant- 3x | 11 | 0 | 3 |
| Protease variant - 6x | 8 | 0 | 6 |
| Protease variant - 12x | 2 | 0 | 12 |
| Integrin/Protease combination (3:3) | 8 | 3 | 3 |
| Integrin/Protease combination (6:6) | 2 | 6 | 6 |
| Integrin/Protease combination (3:9) | 2 | 3 | 3 |
| Integrin/Protease combination (9:3) | 2 | 9 | 3 |

Polymers are synthesized from the three sets of modular genes described in FIG. 6.

Domain 3: C-terminal domain. In the natural production of human collagen, the non-helical C-terminal domain is necessary for correct trimeric assembly, but is subsequently cleaved by procollagen C-proteinase after secretion. Therefore, this domain is incorporated to enable self-assembly of the helical domain, but it is cleaved off with pepsin during the purification procedure. Prior results have shown that pepsin treatment of native procollagen yields only the helical structural region that forms native-type fibrils. Therefore, pepsin similarly affects the collagen-like polymers, resulting in polymers that contain only the triple-helical region (Domain 2).

### Example 2

### Assembly of Synthetic Genes for Human Collagen III and for Variants of Collagen

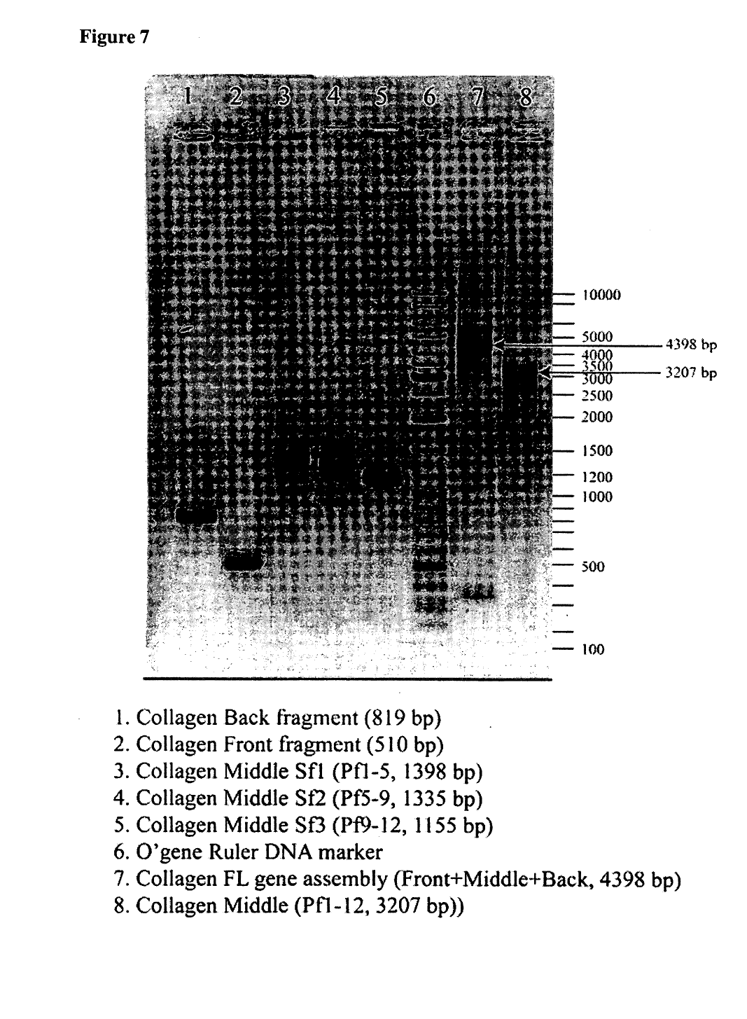

Once the oligonucleotides for Domains 1, 2, and 3 are been designed and optimized using the methods described herein, the genes encoding the biopolymers are synthesized. Domains 1 and 3 is identical for all variants of the collagen-like polymer, and synthesis of these two domains is straightforward since there are no repeating units within the sequence and the domains are relatively short. In vitro PCR assembly is performed as follows. In the first step of assembly, modules approximately 275-bp in length are synthesized by a PCR reaction using 6-7 oligonucleotides. After these fragments are made, a second PCR-assembly step combines the modules together to create the full-length gene. These domains are then cloned into a 2μ yeast expression vector. Domain 2 is first assembled as a library of modules as shown in FIG. 6. The length of these fragments is approximately 260 nucleotides, each assembled from synthetic oligonucleotides. This length is close to the standard lengths used for fragment synthesis in the synthetic gene strategy described herein. Overlaps between adjacent modules are ˜60-70 bp. Depending on the specific gene desired, one module from each segment (1-14) with or without a cell-interacting sequence (e.g., GFOGER) is selected, and PCR assembly is performed. Also, 2-3 unique restriction sites are introduced in Domain 2 during the design of the gene. Because PCR assembly of a gene this size (˜3 kb) with repeating units often proves challenging, the gene is first assembled in smaller sections and then the full-length gene is constructed by ligating the smaller sections together. The full-length gene is analyzed by electrophoresis to determine the success of synthesis, as shown in FIG. 7. FIG. 7 depicts an electrophoretic pattern showing assembly of the full-length collagen III encoding polynucleotide (lane 7) and the collagen middle helical domain encoding polynucleotide (lane 8). The exact nucleotide and amino acid sequences are shown as FIGS. 8-10.

Restriction sites flanking both sides of Domain 2 are introduced during the oligonucleotide design. Therefore, after the synthesis of this domain is performed, it is ligated into the expression vector which contains Domains 1 and 3. Protein expression in yeast and purification of the polymers is then performed according to established protocols.

1. An isolated polynucleotide encoding a collagen-like polypeptide domain, wherein:
at least 50% of the encoded collagen-like polypeptide domain is in the form of Gly-X-Y triads, where X and Y symbolize individual amino acids;
X is proline in at least 20% of said triads;
Y is proline in at least 20% of said triads;
said polynucleotide encodes a region having at least 50 consecutive amino acids, wherein said region is at least 90% identical to an amino acid sequence of a naturally occurring collagen polypeptide; and
no more than 98% of the nucleotides of the collagen-like polypeptide domain-encoding portion of said polynucleotide fall into a window of 100 consecutive nucleotides wherein the nucleotides of said window have greater than 98% identity to a naturally occurring collagen-encoding nucleotide sequence.

2. The isolated nucleic acid sequence encoding a collagen-like polypeptide domain of claim 1, wherein:
said nucleotide sequence is configured to be specifically amplifiable and specifically mutatable.

3. The polynucleotide of claim 1, wherein the encoded polypeptide is capable of assembling into a triple-helical structure upon hydroxylation of said prolines in the Y position of said Gly-X-Y triads.

4. The polynucleotide of claim 1, further comprising at least 2 endonuclease restriction sites not present at the corresponding site in a wild type mammalian collagen-encoding gene sequence.

5. The polynucleotide of claim 1, further comprising at least 2 endonuclease restriction sites distributed throughout said gene at known distances from each other in the nucleotide sequence.

6. The polynucleotide of claim 1, wherein said nucleotide sequence encodes at least 10 consecutive amino acids of a naturally occurring collagen polypeptide core helical domain.

7. The polynucleotide of claim 1, wherein the nucleotide sequence of the collagen-like polypeptide domain-encoding portion of said polynucleotide has less than 70% sequence identity with the nucleotide sequence of naturally-occurring collagen as set forth in SEQ ID NO: 1.

8. The polynucleotide of claim 1, wherein said encoded collagen-like polypeptide domain is at least 100 amino acids in length.

9. The polynucleotide of claim 1, configured for expression in yeast, bacteria, insect or mammalian cells.

10. (canceled)

11. An isolated polypeptide comprising an amino acid sequence at least 70% identical to a naturally occurring collagen polypeptide and comprising more than 3 amino acid changes.

12. The polypeptide of claim 11, wherein said amino acid changes are each independently selected from the group consisting of: a protease recognition site, an integrin binding site, a kinase target site, growth factor binding site, cell binding site, and chemical attachment site.

13. An isolated polypeptide comprising a collagen-like polypeptide domain, wherein:
at least 50% of the encoded collagen-like polypeptide domain is in the form of Gly-X-Y triads, where X and Y symbolize individual amino acids;
X is proline in at least 20% of said triads;
Y is proline in at least 20% of said triads; and
said collagen-like polypeptide domain is at least 300 amino acids in length.

14. A synthetic DNA molecule encoding the polypeptide of claim 11, wherein said DNA molecule has no more than 99% sequence identity to the naturally occurring nucleotide sequence encoding said collagen polypeptide.

15. The synthetic DNA molecule of claim 14 further encoding a collagen C- or N-terminal propeptide.

16. (canceled)

17. The synthetic DNA molecule of claim 14 to which a signal sequence is attached.

18. The synthetic DNA molecule of claim 14, wherein said mature, full-length collagen polypeptide is human collagen I, II, III, IV, V or XI.

19. The synthetic DNA molecule of claim 14, wherein said synthetic DNA molecule has a nucleotide sequence selected from the group consisting of SEQ ID NOs: 3, 5, 7, 9 and 11.

20. A method of making the synthetic DNA molecule of claim 14, comprising:
synthesizing two or more polynucleotide fragments of said synthetic DNA molecule; and
assembling said two or more polynucleotide fragments into the full-length synthetic DNA molecule.

21. A method of making a vector encoding collagen, comprising introducing the synthetic DNA of claim 14 into a vector, wherein said introduced synthetic DNA is operatively linked to a regulatory sequence.

22. The polynucleotide of claim 1, wherein said polynucleotide further encodes a non-triple helical domain from a naturally occurring collagen.

23. (canceled)
